(19) United States
(12) Patent Application Publication
(10) Pub. No.: US 2024/0165411 A1
(43) Pub. Date: May 23, 2024

(54) METHODS FOR ANALYZING RESPIRATION AND SLEEP STATE

(71) Applicant: XII Medical, Inc., Union City, CA (US)

(72) Inventors: Anthony V. Caparso, North Ridgeville, OH (US); Praveen Kakumanu, San Ramon, CA (US); Srinivasamurthy Ravi Prakash, Saratoga, CA (US); Bhavesh Patel, Union City, CA (US); Javier Echauz, Alpharetta, GA (US)

(21) Appl. No.: 18/516,832

(22) Filed: Nov. 21, 2023

Related U.S. Application Data
(60) Provisional application No. 63/384,846, filed on Nov. 23, 2022.
(60) Provisional application No. 63/478,445, filed on Jan. 4, 2023.

(51) Int. Cl. A61N 1/36; A61B 5/00; A61B 5/08; A61B 5/389

(52) U.S. Cl. CPC A61N 1/3611; A61B 5/08; A61B 5/389; A61B 5/6847; A61N 1/36139

(57) ABSTRACT

Methods for analyzing respiration and sleep state of a patient are disclosed herein. Various embodiments of the present technology relate to methods for respiratory analysis. In some embodiments, a method comprises obtaining EMG data using a sensor implanted in a sublingual region of a patient. The EMG data can comprise an EMG waveform indicative of activity of a muscle. The method can further comprise determining an envelope of the EMG waveform, determining an inspiration onset of an upcoming respiratory cycle of the patient, based on the envelope of the EMG waveform, and delivering stimulation energy to a hypoglossal nerve of the patient before the inspiration onset. Alternatively or in combination, embodiments of the present technology can include methods for sleep state detection, sleep disordered breathing event detection, and/or sleep position detection.

### CROSS-REFERENCE TO RELATED APPLICATION(S)

The present application claims the benefit of priority to U.S. Provisional Patent Application No. 63/478,445, filed Jan. 4, 2023, titled METHODS FOR ANALYZING RESPIRATION AND SLEEP STATE, and U.S. Provisional Patent Application No. 63/384,846, filed Nov. 23, 2022, titled METHODS FOR ANALYZING RESPIRATION AND SLEEP STATE, each of which is incorporated by reference herein in its entirety.

The present application is also related to U.S. patent application Ser. No. 16/507,390, filed Jul. 10, 2019, titled SYSTEM AND METHOD FOR TREATING OBSTRUCTIVE SLEEP APNEA, and U.S. Pat. No. 11,420,063, filed May 4, 2020, titled SYSTEMS AND METHODS TO IMPROVE SLEEP DISORDERED BREATHING USING CLOSED-LOOP FEEDBACK, each of which is incorporated by reference herein in its entirety.

### TECHNICAL FIELD

The present technology relates to methods for analyzing respiration and sleep state of a patient.

### BACKGROUND

Sleep disordered breathing (SDB), such as upper airway sleep disorders (UASDs), is a condition that occurs that diminishes sleep time and sleep quality, resulting in patients exhibiting symptoms that include daytime sleepiness, tiredness, and lack of concentration. Obstructive sleep apnea (OSA), the most common type of SDB, affects one in five adults in the United States. One in 15 adults has moderate to severe OSA and requires treatment. Untreated OSA results in reduced quality of life measures and increased risk of disease, including hypertension, stroke, heart disease, and others.

OSA is characterized by the complete obstruction of the airway, causing breathing to cease completely (apnea) or partially (hypopnea). During sleep, the tongue muscles relax. In this relaxed state, the tongue may lack sufficient muscle tone to prevent the tongue from changing its normal tonic shape and position. When the base of the tongue and/or soft tissue of the upper airway collapse, the upper airway channel is blocked, causing an apnea event. Blockage of the upper airway prevents air from flowing into the lungs, thereby decreasing the patient's blood oxygen level, which in turn increases blood pressure and heart dilation. This causes a reflexive forced opening of the upper airway channel until normal patency is regained, followed by normal respiration until the next apneic event. These reflexive forced openings briefly arouse the patient from sleep.

Current treatment options range from drug intervention, non-invasive approaches, to more invasive surgical procedures. In many of these instances, patient acceptance and therapy compliance are well below desired levels, rendering the current solutions ineffective as a long-term solution. Continuous positive airway pressure (CPAP), for example, is a standard treatment for OSA. While CPAP is non-invasive and highly effective, it is not well tolerated by all patients and has several side effects. Patient compliance and/or tolerance for CPAP is often reported to be between 40% and 60%. Surgical treatment options for OSA, such as anterior tongue muscle repositioning, orthognathic bimaxillary advancement, uvula-palatalpharyngoplasty, and tracheostomy are available too. However, these procedures tend to be highly invasive, irreversible, and have poor and/or inconsistent efficacy. Even the more effective surgical procedures are undesirable because they usually require multiple invasive and irreversible operations, they may alter a patient's appearance (e.g., maxillo-mandibular advancement), and/or they may be socially stigmatic (e.g., tracheostomy) and have extensive morbidity.

### SUMMARY

The present technology is illustrated, for example, according to various aspects described below, including with reference to the Figures. Various examples of aspects of the present technology are described as numbered examples (1, 2, 3, etc.) for convenience. These are provided as examples and do not limit the subject technology.

Example 1. A method comprising:
obtaining EMG data using a sensor implanted in a sublingual region of a patient, wherein the EMG data comprises an EMG waveform indicative of activity of a muscle;
determining an envelope of the EMG waveform;
determining an inspiration onset of an upcoming respiratory cycle of the patient, based on the envelope of the EMG waveform; and
delivering stimulation energy to a hypoglossal nerve of the patient before the inspiration onset.

Example 2. The method of example 1, wherein the muscle comprises an anterior lingual muscle.

Example 3. The method of example 2, wherein the anterior lingual muscle comprises one or more of a genioglossus muscle or a geniohyoid muscle.

Example 4. The method of any one of examples 1 to 3, wherein determining the inspiration onset comprises detecting a precursor to the inspiration onset, based on the envelope of the EMG waveform.

Example 5. The method of example 4, wherein the precursor comprises phasic activity of the muscle occurring before the inspiration onset.

Example 6. The method of example 4 or 5, wherein the precursor to the inspiration onset is detected by evaluating whether a magnitude of the envelope of the EMG waveform is greater than a threshold value.

Example 7. The method of any one of examples 4 to 6, wherein the precursor to the inspiration onset is detected by evaluating a rate of change of the envelope of the EMG waveform.

Example 8. The method of any one of examples 4 to 7, wherein the precursor to the inspiration onset is detected by:
extracting a set of features from the envelope of the EMG waveform, and
inputting the set of features into a trained machine learning model.

Example 9. The method of any one of examples 4 to 8, wherein the stimulation energy is delivered in response to detecting the precursor to the inspiration onset.

Example 10. The method of any one of examples 1 to 9, further comprising detecting an expiration onset, based on the envelope of the EMG waveform.

Example 11. The method of example 10, wherein the expiration onset is detected by evaluating whether a magnitude of the envelope of the EMG waveform is less than a threshold value.

Example 12. The method of example 10 or 11, wherein the expiration onset is detected by evaluating a rate of change of the envelope of the EMG waveform.

Example 13. The method of any one of examples 10 to 12, wherein the expiration onset is detected by:
extracting a set of features from the envelope of the EMG waveform, and
inputting the set of features into a trained machine learning model.

Example 14. The method of any one of examples 10 to 13, further comprising identifying a candidate breath, based on the inspiration onset and the expiration onset.

Example 15. The method of example 14, further comprising validating the candidate breath.

Example 16. The method of example 15, wherein the candidate breath is validated by:
extracting a set of features from one or more of the EMG waveform or the envelope of the EMG waveform, and
inputting the set of features into a trained machine learning model.

Example 17. The method of any one of examples 1 to 16, wherein determining the inspiration onset comprises predicting the inspiration onset, based on the envelope of the EMG waveform and using a breath prediction algorithm.

Example 18. The method of example 17, wherein the breath prediction algorithm is configured to predict the inspiration onset based on previous EMG data of at least one previous respiratory cycle of the patient.

Example 19. The method of example 18, wherein the breath prediction algorithm is configured to:
determine a time parameter for the at least one previous respiratory cycle, based on the previous EMG data, and
predict a time of the inspiration onset of the upcoming respiratory cycle based on the time parameter.

Example 20. The method of example 19, wherein the time parameter for the at least one previous respiratory cycle comprises one or more of the following: an inspiration onset time, an inspiration end time, an inspiration lag time, an expiration onset time, an expiration end time, an expiration lag time, or an inter-breath interval.

Example 21. The method of any one of examples 18 to 20 wherein the breath prediction algorithm is configured to:
detect a candidate breath of the at least one previous respiratory cycle,
determine whether the candidate breath was a valid breath, and
if the candidate breath was a valid breath, predict the inspiration onset based on a time parameter of the candidate breath.

Example 22. The method of any one of examples 17 to 21, wherein the breath prediction algorithm comprises a trained machine learning model.

Example 23. The method of example 22, wherein predicting the inspiration onset comprises inputting at least one feature extracted from the EMG waveform into the trained machine learning model.

Example 24. The method of example 23, wherein the at least one feature extracted from the EMG waveform comprises one or more of the following: a value of the EMG waveform, a magnitude of the EMG waveform, an amplitude of the EMG waveform, a frequency of the EMG waveform, a threshold crossing of the EMG waveform, a complexity of the EMG waveform, a range of the EMG waveform, a variance of the EMG waveform, a transform of the EMG waveform, a statistical parameter of the EMG waveform, or a temporal feature of the EMG waveform.

Example 25. The method of any one of examples 22 to 24, wherein predicting the inspiration onset comprises inputting at least one feature extracted from the envelope of the EMG waveform into the trained machine learning model.

Example 26. The method of example 25, wherein the at least one feature extracted from the envelope of the EMG waveform comprises one or more of the following: a value of the envelope, a magnitude of the envelope, an amplitude of the envelope, a frequency of the envelope, a threshold crossing of the envelope, a complexity of the envelope, a range of the envelope, a variance of the envelope, a transform of the envelope, a statistical parameter of the envelope, or a temporal feature of the envelope.

Example 27. The method of any one of examples 17 to 26, further comprising:
assessing whether the predicted inspiration onset matched an actual inspiration onset of the upcoming respiratory cycle, and
adjusting the breath prediction algorithm based on the assessment.

Example 28. The method of any one of examples 1 to 27, wherein the inspiration onset is determined using at least trained machine learning model that is trained on previous data of the patient.

Example 29. The method of any one of examples 1 to 28, wherein the inspiration onset is determined using at least one trained machine learning model that is trained on data of one or more other patients.

Example 30. The method of any one of examples 1 to 29, wherein the stimulation energy is delivered at least 0.5 microseconds before the inspiration onset.

Example 31. The method of any one of examples 1 to 30, wherein the stimulation energy is delivered no more than 150 milliseconds before the inspiration onset.

Example 32. The method of any one of examples 1 to 31, further comprising:
detecting a sleep state of the patient, based on the EMG data, and
adjusting the stimulation energy based on the sleep state.

Example 33. The method of any one of examples 1 to 32, further comprising:
detecting an apnea or hypopnea event of the patient, based on the EMG data, and
adjusting the stimulation energy based on the apnea or hypopnea event.

Example 34. The method of any one of examples 1 to 32, further comprising:
detecting a position of the patient, based on the EMG data, and
adjusting the stimulation energy based on the position of the patient.

Example 35. The method of any one of examples 1 to 33, wherein the stimulation energy is delivered using an electrode implanted in the sublingual region of the patient.

Example 36. A system comprising:
a sensor configured to be implanted in a sublingual region of a patient;
an electrode configured to be implanted adjacent to a hypoglossal nerve of the patient and configured to deliver stimulation energy to the hypoglossal nerve;
one or more processors; and
a memory operably coupled to the one or more processors and storing instructions that, when executed by the processor, cause the system to perform operations comprising:
   obtaining EMG data using the sensor, wherein the EMG data comprises an EMG waveform indicative of activity of a muscle,
   determining an envelope of the EMG waveform,
   determining an inspiration onset of an upcoming respiratory cycle of the patient, based on the envelope of the EMG waveform, and
   delivering the stimulation energy via the electrode to the hypoglossal nerve before the inspiration onset.

Example 37. The system of example 36, wherein determining the inspiration onset comprises detecting a precursor to the inspiration onset, based on the envelope of the EMG waveform.

Example 38. The system of example 37, wherein the precursor comprises phasic activity of the muscle occurring before the inspiration onset.

Example 39. The system of example 37 or 38, wherein the precursor to the inspiration onset is detected by evaluating whether a magnitude of the envelope of the EMG waveform is greater than a threshold value.

Example 40. The system of any one of examples 37 to 39, wherein the precursor to the inspiration onset is detected by evaluating a rate of change of the envelope of the EMG waveform.

Example 41. The system of any one of examples 37 to 40, wherein the precursor to the inspiration onset is detected by:
extracting a set of features from the envelope of the EMG waveform, and
inputting the set of features into a trained machine learning model.

Example 42. The system of any one of examples 37 to 41, wherein the stimulation energy is delivered in response to detecting the precursor to the inspiration onset.

Example 43. The system of any one of examples 36 to 42, wherein the operations further comprise detecting an expiration onset, based on the envelope of the EMG waveform.

Example 44. The system of example 43, wherein the expiration onset is detected by evaluating whether a magnitude of the envelope of the EMG waveform is less than a threshold value.

Example 45. The system of example 43 or 44, wherein the expiration onset is detected by evaluating a rate of change of the envelope of the EMG waveform.

Example 46. The system of any one of examples 43 to 45, wherein the expiration onset is detected by:
extracting a set of features from the envelope of the EMG waveform, and
inputting the set of features into a trained machine learning model.

Example 47. The system of any one of examples 43 to 46, wherein the operations further comprise identifying a candidate breath, based on the inspiration onset and the expiration onset.

Example 48. The system of example 47, wherein the operations further comprise validating the candidate breath.

Example 49. The system of example 48, wherein the candidate breath is validated by:
extracting a set of features from one or more of the EMG waveform or the envelope of the EMG waveform, and
inputting the set of features into a trained machine learning model.

Example 50. The system of any one of examples 36 to 49, wherein determining the inspiration onset comprises predicting the inspiration onset, based on the envelope of the EMG waveform and using a breath prediction algorithm.

Example 51. The system of example 50, wherein the breath prediction algorithm is configured to predict the inspiration onset based on previous EMG data of at least one previous respiratory cycle of the patient.

Example 52. The system of example 51, wherein the breath prediction algorithm is configured to:
determine a time parameter for the at least one previous respiratory cycle, based on the previous EMG data, and
calculate a time of the inspiration onset of the upcoming respiratory cycle based on the time parameter.

Example 53. The system of example 52, wherein the time parameter for the at least one previous respiratory cycle comprises one or more of the following: an inspiration onset time, an inspiration end time, an inspiration lag time, an expiration onset time, an expiration end time, an expiration lag time, or an inter-breath interval.

Example 54. The system of any one of examples 51 to 53 wherein the breath prediction algorithm is configured to:
detect a candidate breath of the at least one previous respiratory cycle,
determine whether the candidate breath was a valid breath, and
if the candidate breath was a valid breath, predict the inspiration onset based on a time parameter of the candidate breath.

Example 55. The system of any one of examples 50 to 54, wherein the breath prediction algorithm comprises a trained machine learning model.

Example 56. The system of example 55, wherein predicting the inspiration onset comprises inputting at least one feature extracted from the EMG waveform into the trained machine learning model.

Example 57. The system of example 56, wherein the at least one feature extracted from the EMG waveform comprises one or more of the following: a value of the EMG waveform, a magnitude of the EMG waveform, an amplitude of the EMG waveform, a frequency of the EMG waveform, a threshold crossing of the EMG waveform, a complexity of the EMG waveform, a range of the EMG waveform, a variance of the EMG waveform, a transform of the EMG waveform, a statistical parameter of the EMG waveform, or a temporal feature of the EMG waveform.

Example 58. The system of any one of examples 55 to 57, wherein predicting the inspiration onset comprises inputting at least one feature extracted from the envelope of the EMG waveform into the trained machine learning model.

Example 59. The system of example 58, wherein the at least one feature extracted from the envelope of the EMG waveform comprises one or more of the following: a value of the envelope, a magnitude of the envelope, an amplitude of the envelope, a frequency of the envelope, a threshold crossing of the envelope, a complexity of the envelope, a range of the envelope, a variance of the envelope, a transform of the envelope, a statistical parameter of the envelope, or a temporal feature of the envelope.

Example 60. The system of any one of examples 50 to 59, wherein the operations further comprise:
assessing whether the predicted inspiration onset matched an actual inspiration onset of the upcoming respiratory cycle, and
adjusting the breath prediction algorithm based on the assessment.

Example 61. The system of any one of examples 50 to 60, wherein the operations further comprise updating the breath prediction algorithm, based on data received from a device external to the patient.

Example 62. The system of example 61, wherein the device is or is communicably coupled to a cloud server.

Example 63. The system of any one of examples 36 to 62, wherein the inspiration onset is determined using at least one trained machine learning model that is trained on previous data of the patient.

Example 64. The system of any one of examples 36 to 63, wherein the inspiration onset is determine using at least one trained machine learning model that is trained on data of one or more other patients.

Example 65. The system of any one of examples 36 to 64, wherein the stimulation energy is delivered at least 0.5 microseconds before the inspiration onset.

Example 66. The system of any one of examples 36 to 65, wherein the stimulation energy is delivered no more than 150 milliseconds before the inspiration onset.

Example 67. The system of any one of examples 36 to 66, wherein the operations further comprise:
detecting a sleep state of the patient, based on the EMG data, and
adjusting the stimulation energy based on the sleep state.

Example 68. The system of any one of examples 36 to 67, wherein the operations further comprise:
detecting an apnea or hypopnea event of the patient, based on the EMG data, and
adjusting the stimulation energy based on the apnea or hypopnea event.

Example 69. The system of any one of examples 36 to 68, wherein the operations further comprise:
detecting a position of the patient, based on the EMG data, and
adjusting the stimulation energy based on the position of the patient.

Example 70. The system of any one of examples 36 to 69, wherein the one or more processors and the memory are located onboard a device implanted in the patient.

Example 71. The system of example 70, wherein the device comprises the sensor and the electrode.

Example 72. A non-transitory computer-readable storage medium comprising instructions that, when executed by one or more processors of a computing system, cause the computing system to perform operations comprising:
obtaining EMG data using a sensor implanted in a sublingual region of a patient, wherein the EMG data comprises a EMG waveform indicative of activity of a muscle;
determining an envelope of the EMG waveform;
determining an inspiration onset of an upcoming respiratory cycle of the patient, based on the envelope of the EMG waveform; and
delivering stimulation energy to a hypoglossal nerve of the patient before the inspiration onset.

Example 73. A method comprising:
obtaining EMG data using a sensor implanted in a sublingual region of a patient, wherein the EMG data comprises an EMG waveform indicative of activity of a muscle;
determining an envelope of the EMG waveform;
generating a set of features from the envelope of the EMG waveform;
identifying a sleep state of the patient, based on the set of features and using a sleep state detection algorithm; and
delivering stimulation energy to a hypoglossal nerve of the patient, based on the sleep state.

Example 74. The method of example 73, wherein the sleep state comprises one or more of the following: whether the patient is asleep, whether patient is awake, N1 sleep, N2 sleep, N3 sleep, non-REM sleep, or REM sleep.

Example 75. The method of example 73 or 74, wherein the muscle comprises an anterior lingual muscle of the patient.

Example 76. The method of example 75, wherein the anterior lingual muscle comprises one or more of a genioglossus muscle or a geniohyoid muscle.

Example 77. The method of any one of examples 73 to 76, wherein the sleep state detection algorithm is configured to identify activity of the muscle indicative of the sleep state.

Example 78. The method of any one of examples 73 to 77, wherein the sleep state detection algorithm is configured to identify a pattern in one or more of the EMG waveform or the envelope of the EMG waveform indicative of the sleep state.

Example 79. The method of any one of examples 73 to 78, wherein the sleep state detection algorithm comprises a trained machine learning model.

Example 80. The method of example 79, wherein the trained machine learning model is trained on previous data of the patient.

Example 81. The method of example 79 or 80, wherein the trained machine learning model is trained on data of one or more other patients.

Example 82. The method of any one of examples 73 to 81, wherein the set of features comprises one or more of the following: a value of the envelope, a magnitude of the envelope, an amplitude of the envelope, a frequency of the envelope, a threshold crossing of the envelope, a complexity of the envelope, a range of the envelope, a variance of the envelope, a transform of the envelope, a statistical parameter of the envelope, or a temporal feature of the envelope.

Example 83. The method of any one of examples 73 to 82, wherein the sleep state is identified based on a set of second features comprising one or more of the following: a value of the EMG waveform, a magnitude of the EMG waveform, an amplitude of the EMG waveform, a frequency of the EMG waveform, a threshold crossing of the EMG waveform, a complexity of the EMG waveform, a range of the EMG waveform, a variance of the EMG waveform, a transform of the EMG waveform, a statistical parameter of the EMG waveform, or a temporal feature of the EMG waveform.

Example 84. The method of example 83, further comprising pre-processing the EMG data to generate cleaned EMG data, wherein the set of second features are generated based on the cleaned EMG data.

Example 85. The method of any one of examples 73 to 84, further comprising adjusting at least one parameter of the stimulation energy based on the sleep state, wherein the at least one parameter comprises one or more of the following: amplitude, pulse width, duty cycle, polarity, frequency, ramp up time, ramp down time, ramp up rate, ramp down rate, or waveform.

Example 86. The method of any one of examples 73 to 85, further comprising predicting an inspiration onset of an upcoming respiratory cycle of the patient, based on the EMG data and using a breath prediction algorithm.

Example 87. The method of example 86, wherein the stimulation energy is delivered before the predicted inspiration onset.

Example 88. The method of any one of examples 73 to 87, wherein the stimulation energy is delivered using an electrode implanted in the sublingual region of the patient.

Example 89. A system comprising:
a sensor configured to be implanted in a sublingual region of a patient;
an electrode configured to be implanted adjacent to a hypoglossal nerve of the patient and configured to deliver stimulation energy to the hypoglossal nerve;
one or more processors; and
a memory operably coupled to the one or more processors and storing instructions that, when executed by the processor, cause the system to perform operations comprising:
   obtaining EMG data using the sensor, wherein the EMG data comprises an EMG waveform indicative of activity of a muscle,
   determining an envelope of the EMG waveform,
   generating a set of features from the envelope of the EMG waveform,
   identifying a sleep state of the patient, based on the set of features and using a sleep state detection algorithm, and
   delivering the stimulation energy via the electrode to the hypoglossal nerve, based on the sleep state.

Example 90. The system of example 89, wherein the sleep state comprises one or more of the following: whether the patient is asleep, whether patient is awake, N1 sleep, N2 sleep, N3 sleep, or REM sleep.

Example 91. The system of example 89 or 90, wherein the sleep state detection algorithm is configured to identify activity of the muscle indicative of the sleep state.

Example 92. The system of any one of examples 89 to 91, wherein the sleep state detection algorithm is configured to identify a pattern in one or more of the EMG waveform or the envelope of the EMG waveform indicative of the sleep state.

Example 93. The system of any one of examples 89 to 92, wherein the sleep state detection algorithm comprises a trained machine learning model.

Example 94. The system of example 93, wherein the trained machine learning model is trained on previous data of the patient.

Example 95. The system of example 93 or 94, wherein the trained machine learning model is trained on data of one or more other patients.

Example 96. The system of any one of examples 89 to 95, wherein the set of features comprises one or more of the following: a value of the envelope, a magnitude of the envelope, an amplitude of the envelope, a frequency of the envelope, a threshold crossing of the envelope, a complexity of the envelope, a range of the envelope, a variance of the envelope, a transform of the envelope, a statistical parameter of the envelope, or a temporal feature of the envelope.

Example 97. The system of any one of examples 89 to 96, wherein the sleep state is identified based on a set of second features comprising one or more of the following: a value of the EMG waveform, a magnitude of the EMG waveform, an amplitude of the EMG waveform, a frequency of the EMG waveform, a threshold crossing of the EMG waveform, a complexity of the EMG waveform, a range of the EMG waveform, a variance of the EMG waveform, a transform of the EMG waveform, a statistical parameter of the EMG waveform, or a temporal feature of the EMG waveform.

Example 98. The system of example 97, wherein the operations further comprise pre-processing the EMG data to generate cleaned EMG data, wherein the set of second features are generated based on the cleaned EMG data.

Example 99. The system of any one of examples 89 to 98, wherein the operations further comprise adjusting at least one parameter of the stimulation energy based on the sleep state, wherein the at least one parameter comprises one or more of the following: amplitude, pulse width, duty cycle, polarity, frequency, ramp up time, ramp down time, ramp up rate, ramp down rate, or waveform.

Example 100. The system of any one of examples 89 to 99, wherein the operations further comprise predicting an inspiration onset of an upcoming respiratory cycle of the patient, based on the EMG data and using a breath prediction algorithm.

Example 101. The system of example 100, wherein the stimulation energy is delivered before the predicted inspiration onset.

Example 102. The system of any one of examples 89 to 101, wherein the one or more processors and the memory are located onboard a device implanted in the patient.

Example 103. The system of example 102, wherein the device comprises the sensor and the electrode.

Example 104. The system of example 102 or 103, wherein the operations further comprise updating the sleep state detection algorithm, based on data received from an external device.

Example 105. A non-transitory computer-readable storage medium comprising instructions that, when executed by one or more processors of a computing system, cause the computing system to perform operations comprising:
obtaining EMG data using a sensor implanted in a sublingual region of a patient, wherein the EMG data comprises an EMG waveform indicative of activity of a muscle;
determining an envelope of the EMG waveform;
generating a set of features from the envelope of the EMG waveform;
identifying a sleep state of the patient, based on the set of features and using a sleep state detection algorithm; and
delivering stimulation energy to a hypoglossal nerve of the patient, based on the sleep state.

Example 106. A method comprising:
obtaining EMG data using a sensor implanted in a sublingual region of a patient, wherein the EMG data comprises an EMG waveform indicative of activity of a muscle;
determining an envelope of the EMG waveform;
generating a set of features from the envelope of the EMG waveform;
detecting a disordered breathing event of the patient, based on the set of features and using a disordered breathing event detection algorithm; and
delivering stimulation energy to a hypoglossal nerve of the patient, based on the disordered breathing event.

Example 107. The method of example 106, wherein the disordered breathing event comprises one or more of an apnea or a hypopnea.

Example 108. The method of example 106 or 107, wherein the muscle comprises an anterior lingual muscle of the patient.

Example 109. The method of example 108, wherein the anterior lingual muscle comprises one or more of a genioglossus muscle or a geniohyoid muscle.

Example 110. The method of any one of examples 106 to 109, wherein the disordered breathing event detection algorithm is configured to identify activity of the muscle indicative of the disordered breathing event.

Example 111. The method of any one of examples 106 to 110, wherein the disordered breathing event detection algorithm is configured to identify a pattern in one or more of the EMG waveform or the envelope of the EMG waveform indicative of the disordered breathing event.

Example 112. The method of any one of examples 106 to 111, wherein the disordered breathing event detection algorithm comprises a trained machine learning model.

Example 113. The method of example 112, wherein the trained machine learning model is trained on previous data of the patient.

Example 114. The method of example 112 or 113, wherein the trained machine learning model is trained on data of one or more other patients.

Example 115. The method of any one of examples 106 to 114, wherein the set of features comprises one or more of the following: a value of the envelope, a magnitude of the envelope, an amplitude of the envelope, a frequency of the envelope, a threshold crossing of the envelope, a complexity of the envelope, a range of the envelope, a variance of the envelope, a transform of the envelope, a statistical parameter of the envelope, or a temporal feature of the envelope.

Example 116. The method of any one of examples 106 to 115, wherein the disordered breathing event is identified based on a set of second features comprising one or more of the following: a value of the EMG waveform, a magnitude of the EMG waveform, an amplitude of the EMG waveform, a frequency of the EMG waveform, a threshold crossing of the EMG waveform, a complexity of the EMG waveform, a range of the EMG waveform, a variance of the EMG waveform, a transform of the EMG waveform, a statistical parameter of the EMG waveform, or a temporal feature of the EMG waveform.

Example 117. The method of example 116, further comprising pre-processing the EMG data to generate cleaned EMG data, wherein the set of second features are generated based on the cleaned EMG data.

Example 118. The method of any one of examples 106 to 117, further comprising adjusting at least one parameter of the stimulation energy based on the disordered breathing event, wherein the at least one parameter comprises one or more of the following: amplitude, pulse width, duty cycle, polarity, frequency, ramp up time, ramp down time, ramp up rate, ramp down rate, or waveform.

Example 119. The method of any one of examples 106 to 118, further comprising predicting an inspiration onset of an upcoming respiratory cycle of the patient, based on the EMG data and using a breath prediction algorithm.

Example 120. The method of example 119, wherein the stimulation energy is delivered before the predicted inspiration onset.

Example 121. The method of any one of examples 106 to 120, wherein the stimulation energy is delivered using an electrode implanted in the sublingual region of the patient.

Example 122. A system comprising:
a sensor configured to be implanted in a sublingual region of a patient;
an electrode configured to be implanted adjacent to a hypoglossal nerve of the patient and configured to deliver stimulation energy to the hypoglossal nerve;
one or more processors; and
a memory operably coupled to the one or more processors and storing instructions that, when executed by the processor, cause the system to perform operations comprising:
obtaining EMG data using the sensor, wherein the EMG data comprises an EMG waveform indicative of activity of a muscle,
determining an envelope of the EMG waveform,
generating a set of features from the envelope of the EMG waveform,
identifying a disordered breathing event of the patient, based on the set of features and using a disordered breathing event detection algorithm, and
delivering the stimulation energy via the electrode to the hypoglossal nerve, based on the disordered breathing event.

Example 123. The system of example 122, wherein the disordered breathing event comprises one or more of an apnea or a hypopnea.

Example 124. The system of example 122 or 123, wherein the disordered breathing event detection algorithm is configured to identify activity of the muscle indicative of the disordered breathing event.

Example 125. The system of any one of examples 122 to 124, wherein the disordered breathing event detection algorithm is configured to identify a pattern in one or more of the EMG waveform or the envelope of the EMG waveform indicative of the disordered breathing event.

Example 126. The system of any one of examples 122 to 125, wherein the disordered breathing event detection algorithm comprises a trained machine learning model.

Example 127. The system of example 126, wherein the trained machine learning model is trained on previous data of the patient.

Example 128. The system of example 126 or 127, wherein the trained machine learning model is trained on data of one or more other patients.

Example 129. The system of any one of examples 122 to 128, wherein the set of features comprises one or more of the following: a value of the envelope, a magnitude of the envelope, an amplitude of the envelope, a frequency of the envelope, a threshold crossing of the envelope, a complexity of the envelope, a range of the envelope, a variance of the envelope, a transform of the envelope, a statistical parameter of the envelope, or a temporal feature of the envelope.

Example 130. The system of any one of examples 122 to 129, wherein the disordered breathing event is identified based on a set of second features comprising one or more of the following: a value of the EMG waveform, a magnitude of the EMG waveform, an amplitude of the EMG waveform, a frequency of the EMG waveform, a threshold crossing of the EMG waveform, a complexity of the EMG waveform, a range of the EMG waveform, a variance of the EMG waveform, a transform of the EMG waveform, a statistical parameter of the EMG waveform, or a temporal feature of the EMG waveform.

Example 131. The system of example 130, wherein the operations further comprise pre-processing the EMG data to generate cleaned EMG data, wherein the set of second features are generated based on the cleaned EMG data.

Example 132. The system of any one of examples 122 to 131, wherein the operations further comprise adjusting at least one parameter of the stimulation energy based on the disordered breathing event, wherein the at least one parameter comprises one or more of the following: amplitude, pulse width, duty cycle, polarity, frequency, ramp up time, ramp down time, ramp up rate, ramp down rate, or waveform.

Example 133. The system of any one of examples 122 to 132, wherein the operations further comprise predicting an inspiration onset of an upcoming respiratory cycle of the patient, based on the EMG data and using a breath prediction algorithm.

Example 134. The system of example 133, wherein the stimulation energy is delivered before the predicted inspiration onset.

Example 135. The system of any one of examples 122 to 134, wherein the one or more processors and the memory are located onboard a device implanted in the patient.

Example 136. The system of example 135, wherein the device comprises the sensor and the electrode.

Example 137. The system of example 135 or 136, wherein the operations further comprise updating the disordered breathing event detection algorithm, based on data received from an external device.

Example 138. A non-transitory computer-readable storage medium comprising instructions that, when executed by one or more processors of a computing system, cause the computing system to perform operations comprising:
obtaining EMG data using a sensor implanted in a sublingual region of a patient, wherein the EMG data comprises an EMG waveform indicative of activity of a muscle;
determining an envelope of the EMG waveform;
generating a set of features from the envelope of the EMG waveform;
identifying a disordered breathing event of the patient, based on the set of features and using a disordered breathing event detection algorithm; and
delivering stimulation energy to a hypoglossal nerve of the patient, based on the disordered breathing event.

Example 139. A method comprising:
obtaining EMG data using a sensor implanted in a sublingual region of a patient, wherein the EMG data comprises an EMG waveform indicative of activity of a muscle;
determining an envelope of the EMG waveform;
generating a set of features from the envelope of the EMG waveform;
detecting a sleep position of the patient, based on the set of features and using a sleep position detection algorithm; and
delivering stimulation energy to a hypoglossal nerve of the patient, based on the sleep position.

Example 140. The method of example 139, wherein the sleep position comprises one or more of the following: a lateral position, a supine position, or a prone position.

Example 141. The method of example 139 or 140, wherein the muscle comprises an anterior lingual muscle of the patient.

Example 142. The method of example 141, wherein the anterior lingual muscle comprises one or more of a genioglossus muscle or a geniohyoid muscle.

Example 143. The method of any one of examples 139 to 142, wherein the sleep position detection algorithm is configured to identify activity of the muscle indicative of the sleep position.

Example 144. The method of any one of examples 139 to 143, wherein the sleep position detection algorithm is configured to identify a pattern in the EMG waveform indicative of the sleep position.

Example 145. The method of any one of examples 139 to 144, wherein the sleep position detection algorithm comprises a trained machine learning model.

Example 146. The method of example 145, wherein the trained machine learning model is trained on previous data of the patient.

Example 147. The method of example 145 or 146, wherein the trained machine learning model is trained on data of one or more other patients.

Example 148. The method of any one of examples 139 to 147, wherein the set of features comprises one or more of the following: a value of the envelope, a magnitude of the envelope, an amplitude of the envelope, a frequency of the envelope, a threshold crossing of the envelope, a complexity of the envelope, a range of the envelope, a variance of the envelope, a transform of the envelope, a statistical parameter of the envelope, or a temporal feature of the envelope.

Example 149. The method of any one of examples 139 to 148, wherein the sleep position is identified based on a set of second features comprising one or more of the following: a value of the EMG waveform, a magnitude of the EMG waveform, an amplitude of the EMG waveform, a frequency of the EMG waveform, a threshold crossing of the EMG waveform, a complexity of the EMG waveform, a range of the EMG waveform, a variance of the EMG waveform, a transform of the EMG waveform, a statistical parameter of the EMG waveform, or a temporal feature of the EMG waveform.

Example 150. The method of example 149, further comprising pre-processing the EMG data to generate cleaned EMG data, wherein the set of second features are generated based on the cleaned EMG data.

Example 151. The method of any one of examples 139 to 150, further comprising adjusting at least one parameter of the stimulation energy based on the sleep position, wherein the at least one parameter comprises one or more of the following: amplitude, pulse width, duty cycle, polarity, frequency, ramp up time, ramp down time, ramp up rate, ramp down rate, or waveform.

Example 152. The method of any one of examples 139 to 151, further comprising predicting an inspiration onset of an upcoming respiratory cycle of the patient, based on the EMG data and using a breath prediction algorithm.

Example 153. The method of example 152, wherein the stimulation energy is delivered before the predicted inspiration onset.

Example 154. The method of any one of examples 139 to 153, wherein the stimulation energy is delivered using an electrode implanted in the sublingual region of the patient.

Example 155. A system comprising:
a sensor configured to be implanted in a sublingual region of a patient;
an electrode configured to be implanted adjacent to a hypoglossal nerve of the patient and configured to deliver stimulation energy to the hypoglossal nerve;
one or more processors; and
a memory operably coupled to the one or more processors and storing instructions that, when executed by the processor, cause the system to perform operations comprising:
   obtaining EMG data using the sensor, wherein the EMG data comprises an EMG waveform indicative of activity of a muscle,
   determining an envelope of the EMG waveform,
   generating a set of features from the envelope of the EMG waveform,
   identifying a sleep position of the patient, based on the set of features and using a sleep position detection algorithm, and
   delivering the stimulation energy via the electrode to the hypoglossal nerve, based on the sleep position.

Example 156. The system of example 155, wherein the sleep position comprises one or more of the following: a lateral position, a supine position, or a prone position.

Example 157. The system of example 155 or 156, wherein the sleep position detection algorithm is configured to identify activity of the muscle indicative of the sleep position.

Example 158. The system of any one of examples 155 to 157, wherein the sleep position detection algorithm is configured to identify a pattern in the EMG waveform indicative of the sleep position.

Example 159. The system of any one of examples 155 to 158, wherein the sleep position detection algorithm comprises a trained machine learning model.

Example 160. The system of example 159, wherein the trained machine learning model is trained on previous data of the patient.

Example 161. The system of example 159 or 160, wherein the trained machine learning model is trained on data of one or more other patients.

Example 162. The system of any one of examples 155 to 161, wherein the set of features comprises one or more of the following: a value of the envelope, a magnitude of the envelope, an amplitude of the envelope, a frequency of the envelope, a threshold crossing of the envelope, a complexity of the envelope, a range of the envelope, a variance of the envelope, a transform of the envelope, a statistical parameter of the envelope, or a temporal feature of the envelope.

Example 163. The system of any one of examples 155 to 162, wherein the sleep position is identified based on a set of second features comprising one or more of the following: a value of the EMG waveform, a magnitude of the EMG waveform, an amplitude of the EMG waveform, a frequency of the EMG waveform, a threshold crossing of the EMG waveform, a complexity of the EMG waveform, a range of the EMG waveform, a variance of the EMG waveform, a transform of the EMG waveform, a statistical parameter of the EMG waveform, or a temporal feature of the EMG waveform.

Example 164. The system of example 163, wherein the operations further comprise pre-processing the EMG data to generate cleaned EMG data, wherein the set of second features are generated based on the cleaned EMG data.

Example 165. The system of any one of examples 155 to 164, wherein the operations further comprise adjusting at least one parameter of the stimulation energy based on the sleep position, wherein the at least one parameter comprises one or more of the following: amplitude, pulse width, duty cycle, polarity, frequency, ramp up time, ramp down time, ramp up rate, ramp down rate, or waveform.

Example 166. The system of any one of examples 155 to 165, wherein the operations further comprise predicting an inspiration onset of an upcoming respiratory cycle of the patient, based on the EMG data and using a breath prediction algorithm.

Example 167. The system of example 166, wherein the stimulation energy is delivered before the predicted inspiration onset.

Example 168. The system of any one of examples 155 to 167, wherein the one or more processors and the memory are located onboard a device implanted in the patient.

Example 169. The system of example 168, wherein the device comprises the sensor and the electrode.

Example 170. The system of example 168 or 169, wherein the operations further comprise updating the sleep position detection algorithm, based on data received from an external device.

Example 171. A non-transitory computer-readable storage medium comprising instructions that, when executed by one or more processors of a computing system, cause the computing system to perform operations comprising:
obtaining EMG data using a sensor implanted in a sublingual region of a patient, wherein the EMG data comprises an EMG waveform indicative of activity of a muscle;
determining an envelope of the EMG waveform;
generating a set of features from the envelope of the EMG waveform;
identifying a sleep position of the patient, based on the set of features and using a sleep position detection algorithm; and
delivering stimulation energy to a hypoglossal nerve of the patient, based on the sleep position.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is a fragmentary midline sagittal view of an upper airway of a human patient.

FIG. 1B is an illustration of the musculature and hypoglossal innervation of the human tongue.

FIG. 1C is a schematic superior view of a distal arborization of right and left hypoglossal nerves of a human patient. The hypoglossal nerves of FIG. 1C are shown as extending anteriorly from the bottom of the page to the top of the page (e.g., from the hyoid bone to the anterior mandible).

FIG. 2A is a schematic illustration of a neuromodulation system configured in accordance with several embodiments of the present technology.

FIG. 2B is a perspective view of a neuromodulation device configured in accordance with several embodiments of the present technology.

FIGS. 2C and 2D are top and side views, respectively, of the neuromodulation device of FIG. 2B.

FIGS. 3A-3F are various views of the neuromodulation device shown in FIGS. 2B-2D implanted in a human patient in accordance with several embodiments of the present technology.

FIG. 4 illustrates an example of respiratory airflow, a waveform characterizing EMG activity of a genioglossus muscle, and an envelope of the EMG waveform during two consecutive respiratory cycles.

FIG. 5 is a flow diagram illustrating a workflow for respiratory and sleep state analysis in accordance with several embodiments of the present technology.

FIG. 6 is a flow diagram illustrating a workflow for processing electromyography data in accordance with several embodiments of the present technology.

FIG. 7 is a flow diagram illustrating a method for respiratory analysis in accordance with several embodiments of the present technology.

FIG. 8 is a flow diagram illustrating another method for respiratory analysis in accordance with several embodiments of the present technology.

FIG. 9 is a flow diagram illustrating yet another method for respiratory analysis in accordance with several embodiments of the present technology.

FIG. 10 is a flow diagram illustrating still another method for respiratory analysis in accordance with several embodiments of the present technology.

FIGS. 11A-11F are graphs illustrating the respiratory analysis of the method of FIG. 10 in accordance with several embodiments of the present technology.

FIG. 12 is a flow diagram illustrating a method for detecting a disordered breathing event in accordance with several embodiments of the present technology.

FIG. 13 is a flow diagram illustrating a method for sleep state analysis in accordance with several embodiments of the present technology.

FIG. 14 is a flow diagram illustrating a method for patient position analysis in accordance with several embodiments of the present technology.

### DETAILED DESCRIPTION

The present disclosure relates to methods for analyzing respiration and/or sleep state of a patient, and associated systems and devices. For example, the methods herein can include (1) predicting a respiratory cycle of the patient (e.g., the onset of inspiration), (2) detecting a disordered breathing event (e.g., apnea, hypopnea, and/or other event associated with a sleep disorder), (3) detecting the current sleep state of the patient (e.g., whether the patient is awake or in rapid eye movement (REM), N1, N2, or N3 sleep), and/or (4) detecting other relevant patient states (e.g., motion, patient position). In some embodiments, the methods herein are used to control the operation of a neuromodulation system implanted in the patient. Neuromodulation systems can be used to provide a variety of electrical therapies, including neuromodulation therapies such as nerve and/or muscle stimulation. Stimulation can induce excitatory or inhibitory neural or muscular activity. Such therapies can be used at various suitable sites within a patient's anatomy. According to some embodiments, the methods and neuromodulation systems of the present technology are used to treat sleep disordered breathing (SDB), including obstructive sleep apnea (OSA) and/or mixed sleep apnea, via neuromodulation of the hypoglossal nerve (HGN).

For the purpose of contextualizing the structure and operation of the neuromodulation systems and devices disclosed herein, some of the relevant anatomy and physiology are first described below. The headings provided herein are for convenience only and do not interpret the scope or meaning of the claimed present technology. Embodiments under any one heading may be used in conjunction with embodiments under any other heading. For example, any of the neuromodulation systems and devices described in connection with Sections II and III can be used to implement the methods described in connection with Section IV.

### I. Anatomy and Physiology

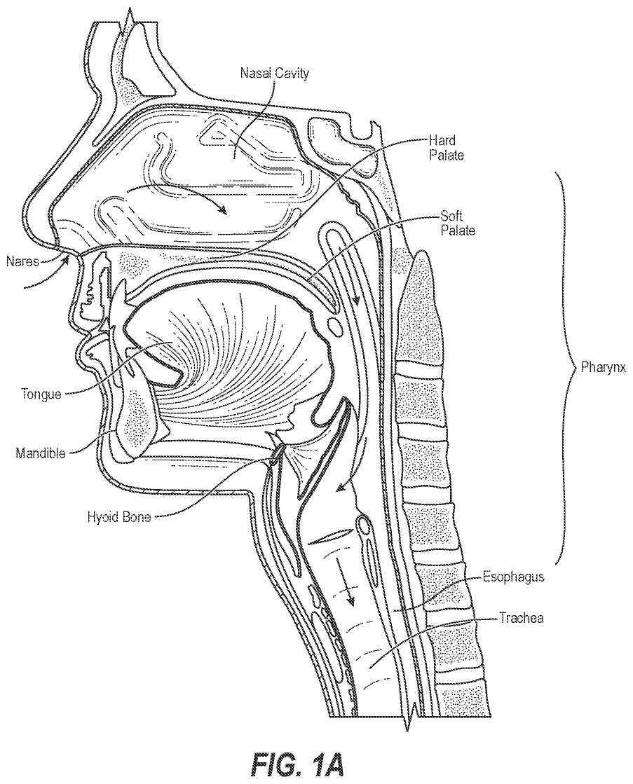

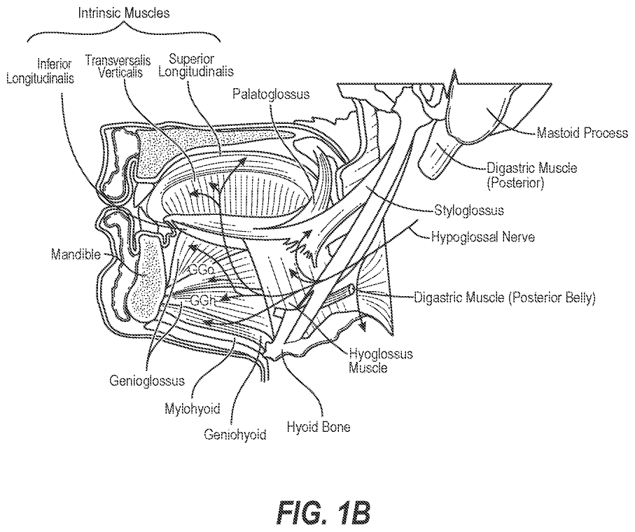

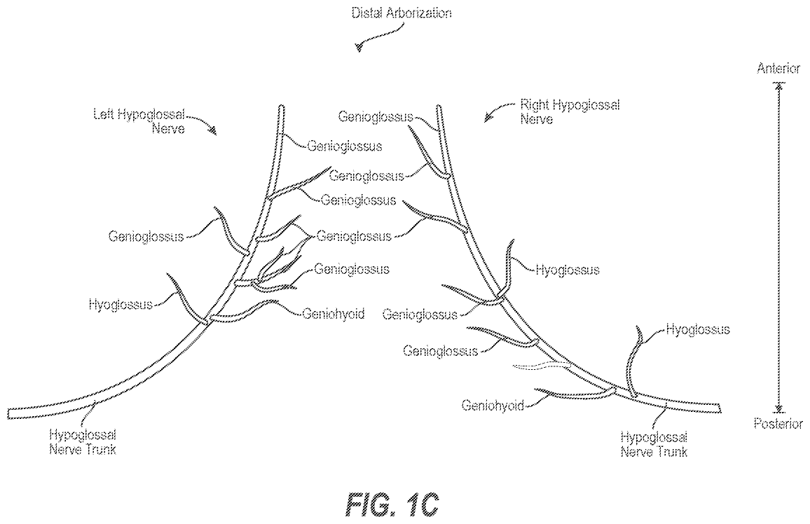

As previously mentioned, respiration in patients with SDB is frustrated due to obstruction, narrowing, and/or collapse of the upper airway during sleep. As shown in FIG. 1A, the upper airway comprises the nasal cavity, the oral cavity, the pharynx, and the larynx. Patency of the upper airway and resistance to airflow in the upper airway are controlled by a complex network of muscles under both voluntary and involuntary neuromuscular control. For example, the muscles of the tongue, the suprahyoid muscles (e.g., the geniohyoid, mylohyoid, stylohyoid, hyoglossus, and the anterior belly of the digastric muscle), and the muscles comprising the soft palate (e.g., palatal muscles) open, widen, and/or stabilize the upper airway during inspiration to counteract the negative airway pressure responsible for drawing air into the airway and the lungs.

With reference to FIG. 1B, the tongue comprises both intrinsic and extrinsic lingual muscles. Generally, activation of the intrinsic muscles changes the shape of the tongue while activation of the extrinsic muscles tends to move the position of the whole tongue. The extrinsic muscles originate at a bony attachment and insert within the tongue. They comprise the genioglossus muscle, the styloglossus muscle, the hyoglossus muscle, and the palatoglossus muscle. The intrinsic muscles both originate and insert within the tongue, and comprise the superior longitudinalis, the inferior longitudinalis, the transversalis, and the verticalis. In a patient who is awake, the brain supplies neural drive to these muscles through the HGN to maintain tongue shape and position, preventing the tongue from blocking the airway.

The lingual muscles are also functionally categorized as either retrusor or protrusor muscles and both intrinsic and extrinsic muscles fall into these categories. The retrusor muscles include the intrinsic superior and inferior longitudinalis muscles and the extrinsic hyoglossus and styloglossus muscles. The protrusor muscles include the intrinsic verticalis and transversalis muscles and the extrinsic genioglossus muscle. Contraction of the styloglossus muscle causes elevation of the tongue while depression of the tongue is the result of downward movements of hyoglossus and genioglossus muscles. Also labeled in FIG. 1B is the geniohyoid muscle, which is a suprahyoid muscle (not a tongue muscle) but still an important protrusor and pharyngeal dilator, and thus contributes to maintaining upper airway patency. It is believed that effective treatment of OSA requires stimulation of the protrusor muscles with minimal or no activation of the retrusor muscles. Thus, for neuromodulation therapy to be effective it is considered beneficial to localize stimulation to the protrusor muscles while avoiding activation of the retrusor muscles.

The largest of the tongue muscles, the genioglossus, comprises two morphological and functional compartments according to fiber distribution, action, and nerve supply. The first, the oblique compartment (GGo), comprises includes vertical fibers that, when contracted, depress the tongue without substantially affecting pharyngeal patency. The second, the horizontal compartment (GGh), contains longitudinal fibers that, when activated, protrude the posterior part of the tongue and enlarge the pharyngeal opening. The GGo contains Type II muscle fibers that are quickly fatigued, whereas the GGh contains Type I muscle fibers that are slower to fatigue. Accordingly, it can be advantageous to stimulate the GGh with little or no stimulation of the GGo to effectively protrude the tongue while preventing or limiting fatigue of the tongue.

The suprahyoid muscles, which comprise the mylohyoid, the geniohyoid, the stylohyoid, and the digastric (only a portion of which is shown in FIG. 1B), extend between the mandible and the hyoid bone to form the floor of the mouth. The geniohyoid is situated inferior to the genioglossus muscle of the tongue and the mylohyoid is situated inferior to the geniohyoid. Contraction of the geniohyoid and tone of the sternohyoid (an infrahyoid muscle, not shown) cooperate to pull the hyoid bone anteriorly to open and/or widen the pharyngeal lumen and stabilize the anterior wall of the hypopharyngeal region. In contrast to the genioglossus and geniohyoid, which are considered tongue protrusors, the hyoglossus and styloglossus are considered tongue retrusors. Activation of the hyoglossus and styloglossus tends to retract the tongue posteriorly, which reduces the size of the pharyngeal opening, increases airway resistance, and frustrates respiration.

As previously mentioned, all of the extrinsic and intrinsic muscles of the tongue are innervated by the HGN, with the exception of the palatoglossus, which is innervated by the vagal nerve. There are two hypoglossal nerves in the body, one on the right side of the head and one on the left side. Each hypoglossal nerve originates at a hypoglossal nucleus in the medulla oblongata of the brainstem, exits the cranium via the hypoglossal canal, and passes inferiorly through the retrostyloid space (a portion of the lateral pharyngeal space) to the occipital artery. The hypoglossal nerve then curves and courses anteriorly to the muscles of the tongue, passing between the anterior edge of the hyoglossus muscle and the posterior edge of the mylohyoid muscle into the sublingual area where it splits into a distal arborization.

FIG. 1C is a schematic superior view of the distal arborization of the right and left hypoglossal nerves. Referring to FIGS. 1B and 1C together, the HGN comprises (1) portions of the distal arborization that innervate the styloglossus and the hyoglossus (tongue retrusor muscles) and (2) portions of the distal arborization that innervate the intrinsic muscles of the tongue, the genioglossus, and the geniohyoid (tongue protrusor muscles). Additionally, the portions of the distal arborization that innervate the tongue retrusor muscles tend to be located posterior of the portions of the distal arborization that innervate the tongue protrusor muscles.

A reduction in activity of the muscles responsible for airway maintenance can result in an increase in airway resistance and a myriad of downstream effects on a patient's respiration and health. Activity of the genioglossus muscle, for example, can decrease during sleep which, whether alone or in combination with other factors (e.g., airway length, airway diameter, soft tissue volume, premature wakening, etc.), can result in substantial airway resistance and/or airway collapse leading to sleep disordered breathing, such as OSA. It is believed that in order for neuromodulation therapy to be effective, it may be beneficial to largely confine stimulation of the HGN to the portions of the distal arborization that innervate protrusor muscles while avoiding or limiting stimulation of the portions of the distal arborization that activate the retrusor muscles.

### II. Neuromodulation Systems

Various embodiments of the present technology are directed to devices, systems, and methods for modulating neurological activity and/or control of one or more nerves associated with one or more muscles involved in airway maintenance. Such neuromodulation can increase activity in targeted muscles, for example the genioglossus and geniohyoid, to reduce a patient's airway resistance and improve the patient's respiration. Moreover, targeted modulation of specific portions of the distal arborization of the hypoglossal nerve can increase activity in tongue protrusor muscles without substantially increasing activity in tongue retrusor muscles to provide a highly efficacious treatment. Additionally or alternatively, targeted modulation of specific portions of the distal arborization of the hypoglossal nerve that innervate the GGh but not portions of the distal arborization of the hypoglossal nerve that innervate the GGo can be used to effectively protrude the tongue while preventing or limiting fatigue of the tongue.

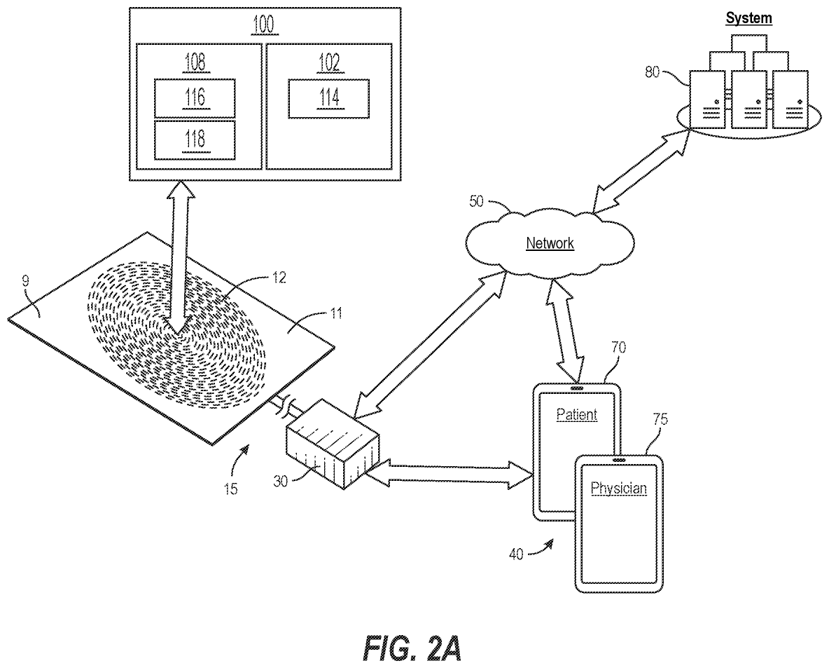

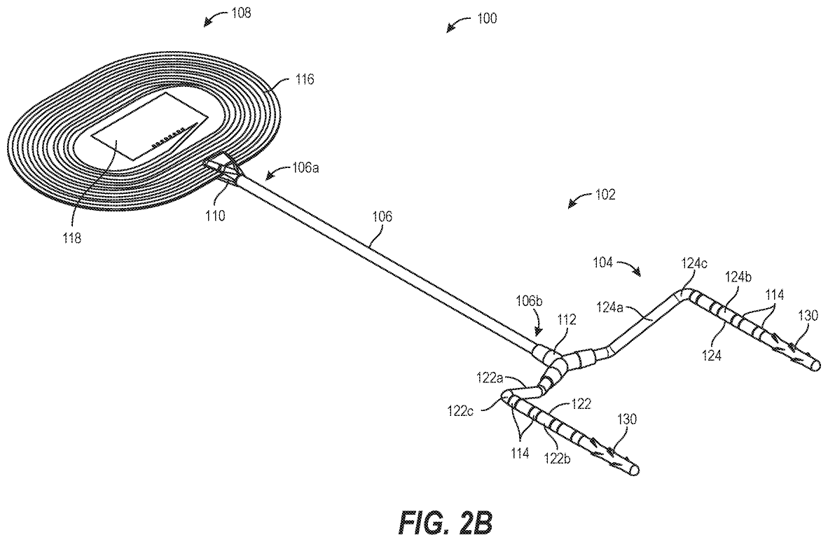

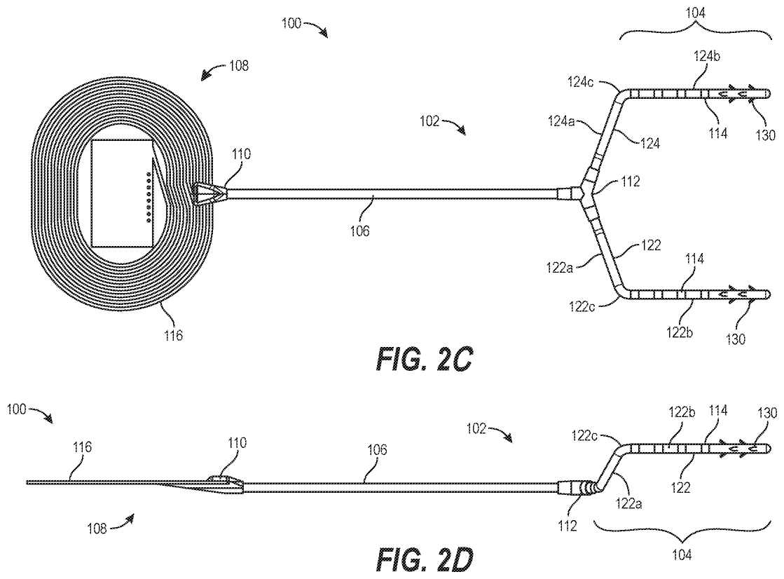

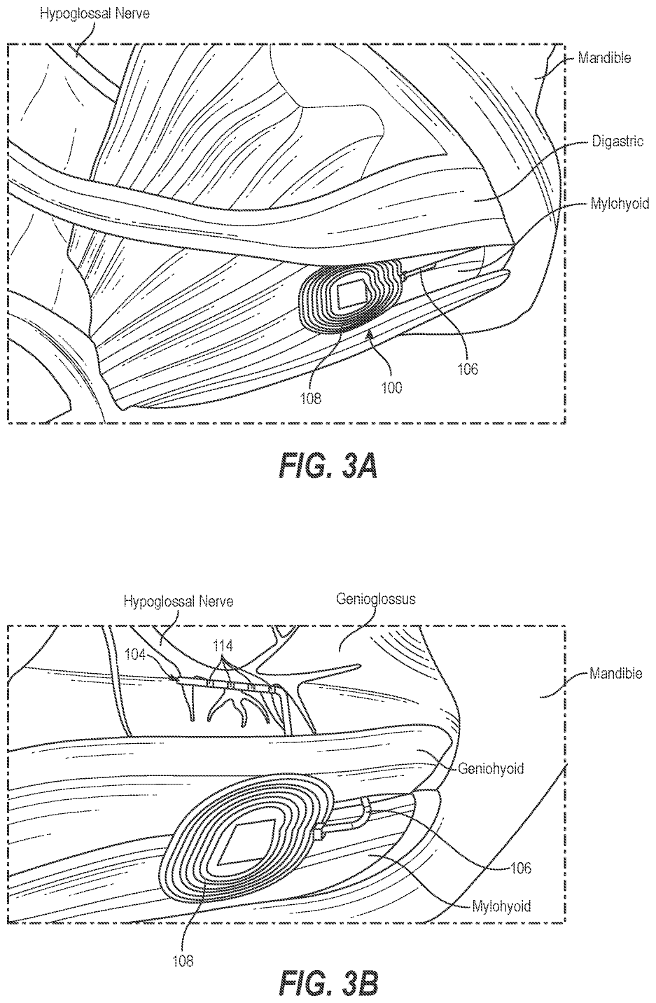

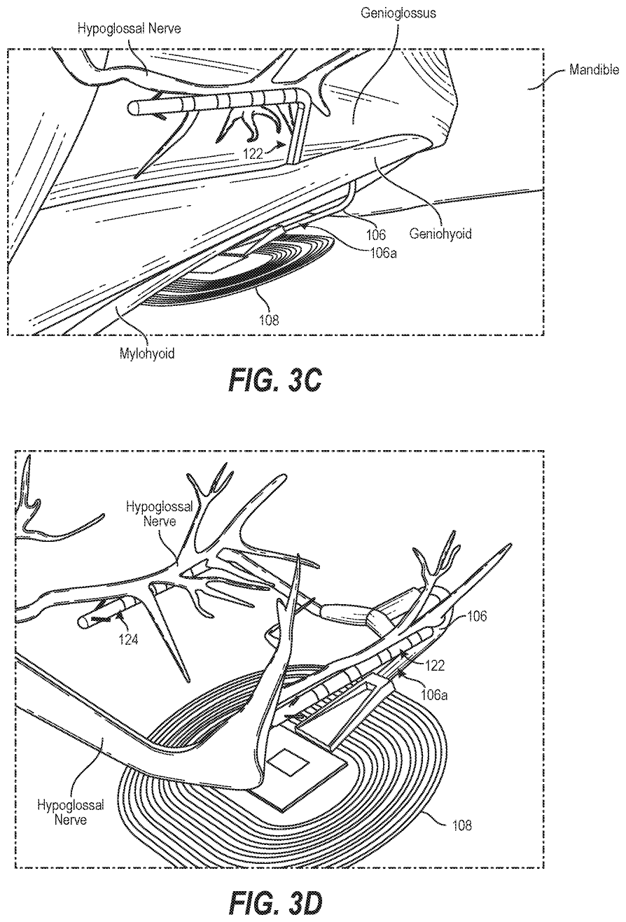

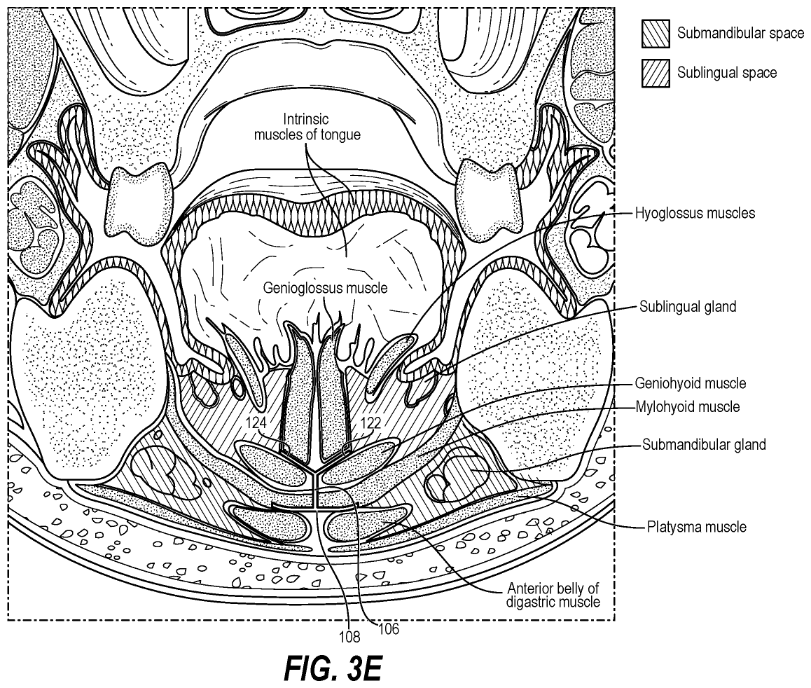

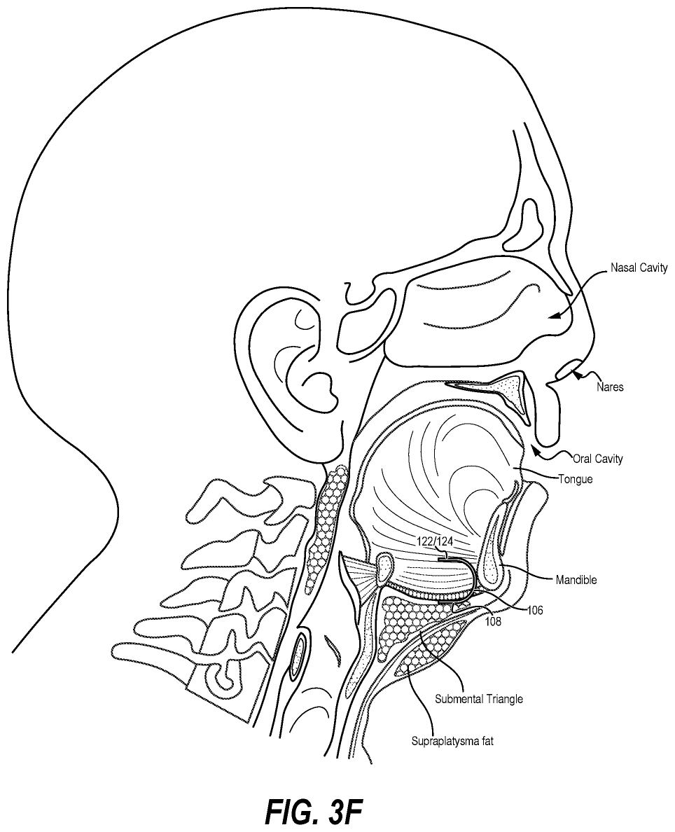

FIG. 2A shows a neuromodulation system 10 for treating SDB configured in accordance with the present technology. The system 10 can include an implantable neuromodulation device 100 and an external system 15 configured to wirelessly couple to the neuromodulation device 100. The neuromodulation device 100 can include a lead 102 having a plurality of conductive elements 114 and an electronics package 108 having a first antenna 116 and an electronics component 118. The neuromodulation device 100 is configured to be implanted at a treatment site comprising submental and sublingual regions of a patient's head, as detailed below with reference to FIGS. 3A-3F.

In use, the electronics package 108 or one or more elements thereof can be configured provide a stimulation energy to the conductive elements 114 that has a pulse width, amplitude, duration, frequency, duty cycle, and/or polarity such that the conductive elements 114 apply an electric field at the treatment site that modulates the hypoglossal nerve. The stimulation energy can be delivered according to a periodic waveform including, for example, a charge-balanced square wave comprising alternating anodic and cathodic pulses.

One or more pulses of the stimulation energy can have a pulse width between about 10 μs and about 1000 μs, between about 50 μs and about 950 μs, between about 100 μs and about 900 μs, between about 150 μs and about 800 μs, between about 200 μs and about 850 μs, between about 250 μs and about 800 μs, between about 300 μs and about 750 μs, between about 350 μs and about 700 μs, between about 400 μs and about 650 μs, between about 450 μs and about 600 μs, between about 500 μs and about 550 μs, about 50 μs, about 100 μs, about 150 μs, about 200 μs, about 250 μs, about 300 μs, about 350 μs, about 400 μs, about 450 μs, about 500 μs, about 550 μs, about 600 μs, about 650 μs, about 700 μs, about 750 μs, about 800 μs, about 850 μs, about 900 μs, about 950 μs, and/or about 1000 μs. In some embodiments, one or more pulses of the stimulation energy has a pulse width of between about 50 μs and about 450 μs.

One or more pulses of the stimulation energy can have an amplitude sufficient to cause an increase in phasic activity of a desired muscle. For example, one or more pulses of the stimulation energy can have a current-controlled amplitude between about 0.1 mA and about 5 mA. In some embodiments, the stimulation energy has an amplitude of about 0.3 mA, about 0.4 mA, about 0.5 mA, about 0.6 mA, about 0.7 mA, about 0.8 mA, about 0.9 mA, about 1 mA, about 1.5 mA, about 2 mA, about 2.5 mA, about 3 mA, about 3.5 mA, about 4 mA, about 4.5 mA, and/or about 5 mA. Additionally or alternatively, an amplitude of one or more pulses of the stimulation energy can be voltage-controlled. An amplitude of one or more pulses of the stimulation energy can be based at least in part on a size and/or configuration of the conductive elements 114, a location of the conductive elements 114 in the patient, etc.

A frequency of the pulses of the stimulation energy can be between about 10 Hz and about 50 Hz, between about 20 Hz and about 40 Hz, about 10 Hz, about 15 Hz, about 20 Hz, about 25 Hz, about 30 Hz, about 35 Hz, about 40 Hz, about 45 Hz, and/or about 50 Hz. In some embodiments, the frequency can be based on a desired effect of the stimulation energy on one or more muscles or nerves. For example, lower frequencies may induce a muscular twitch whereas higher frequencies may include complete contraction of a muscle.

The external system 15 can comprise an external device 11 and a control unit 30 communicatively coupled to the external device 11. In some embodiments, the external device 11 is configured to be positioned proximate a patient's head while they sleep. The external device 11 can comprise a carrier 9 integrated with a second antenna 12. While the control unit 30 is shown separate from the external device 11 in FIG. 2A, in some embodiments the control unit 30 can be integrated with and/or a portion of the external device 11. The second antenna 12 can be configured for multiple purposes. For example, the second antenna 12 can be configured to power the neuromodulation device 100 through electromagnetic induction. Electrical current can be induced in the first antenna 116 when it is positioned above the second antenna 12 of the external device 11, in an electromagnetic field produced by second antenna 12. The first and second antennas 116, 12 can also be configured transmit data to and/or receive data from one another via one or more wireless communication techniques (e.g., Bluetooth, WiFi, USB, etc.) to facilitate communication between the neuromodulation device 100 and the external system 15. This communication can, for example, include programming, e.g., uploading software/firmware revisions to the neuromodulation device 100, changing/adjusting stimulation settings and/or parameters, and/or adjusting parameters of control algorithms.

The control unit 30 of the external system 15 can include a processor and/or a memory that stores instructions (e.g., in the form of software, code or program instructions executable by the processor or controller) for causing the external device to generate an electromagnetic field according to certain parameters provided by the instructions. The external system can include and/or be configured to be coupled to a power source such as a direct current (DC) power supply, an alternating current (AC) power supply, and/or a power supply switchable between DC and AC. The processor of the external system can be used to control various parameters of the energy output by the power source, such as intensity, amplitude, duration, frequency, duty cycle, and polarity. Instead of or in addition to a processor, the external system can include drive circuitry. In such embodiments, the external system can include hardwired circuit elements to provide the desired waveform delivery rather than a software-based generator. The drive circuitry can include, for example, analog circuit elements (e.g., resistors, diodes, switches, etc.) that are configured to cause the power source to supply energy to the second antenna 12 to produce an electromagnetic field according to the desired parameters. In some embodiments, the neuromodulation device 100 can be configured for communication with the external system via inductive coupling.

The system 10 can also include a user interface 40 in the form of a patient device 70 and/or a physician device 75. The user interface(s) 40 can be configured to transmit and/or receive data with the external system 15, the second antenna 12, the control unit 30, the neuromodulation device 100, and/or the remote computing device(s) 80 via wired and/or wireless communication techniques (e.g., Bluetooth, WiFi, USB, etc.). In the example configuration of FIG. 2A, both the patient device 70 and physician device 75 are smartphones. The type of device could, however, vary. One or both of the patient device 70 and physician device 75 can have an application or “app” installed thereon that is user specific, e.g., a patient app or a physician app, respectively. The patient app can allow the patient to execute certain commands necessary for controlling operation of neuromodulation device 100, such as, for example, start/stop therapy, increase/decrease stimulation power or intensity, and/or select a stimulation program. In addition to the controls afforded the patient, the physician app can allow the physician to modify stimulation settings, such as pulse settings (patterns, duration, waveforms, etc.), stimulation frequency, amplitude settings, and electrode configurations, closed-loop and open loop control settings and tuning parameters for the embedded software that controls therapy delivery during use.

The patient and/or physician devices 70, 75 can be configured to communicate with the other components of the system 10 via a network 50. The network 50 can be or include one or more communications networks, such as any of the following: a wired network, a wireless network, a metropolitan area network (MAN), a local area network (LAN), a wide area network (WAN), a virtual local area network (VLAN), an internet, an extranet, an intranet, and/or any other suitable type of network or combinations thereof. The patient and/or physician devices 70, 75 can be configured to communicate with one or more remote computing devices 80 via the network 50 to enable the transfer of data between the devices 70, 75 and the remote computing device(s) 80. Additionally, the external system 15 can be configured to communicate with the other components of the system 10 via the network 50. This can also enable the transfer of data between the external system 15 and remote computing device(s) 80.

The external system 15 can receive the programming, software/firmware, and settings/parameters through any of the communication paths described above, e.g., from the user interface(s) 40 directly (wired or wirelessly) and/or through the network 50. The communication paths can also be used to download data from the neuromodulation device 100, such as measured data regarding completed stimulation therapy sessions, to the external system 15. The external system 15 can transmit the downloaded data to the user interface 40, which can send/upload the data to the remote computing device(s) 80 via the network 50.

In addition to facilitating local control of the system 10, e.g., the external system 15 and the neuromodulation device 100, the various communication paths shown in FIG. 2A can also enable:
Distributing from the remoting computing device(s) 80 software/firmware updates for the patient device 70, physician device 75, external system 15, and/or neuromodulation device 100.
Downloading from the remote computing device(s) 80 therapy settings/parameters to be implemented by the patient device 70, physician device 75, external system 15, and/or neuromodulation device 100.
Facilitating therapy setting/parameter adjustments/algorithm adjustments by a remotely located physician.
Uploading data recorded during therapy sessions.
Maintaining coherency in the settings/parameters by distributing changes and adjustments throughout the system components.

The therapeutic approach implemented with the system 10 can involve implanting only the neuromodulation device 100 and leaving the external system 15 as an external component to be used only during the application of therapy. To facilitate this, the neuromodulation device 100 can be configured to be powered by the external system 15 through electromagnetic induction. In operation, the second antenna 12, operated by control unit 30, can be positioned external to the patient in the vicinity of the neuromodulation device 100 such that the second antenna 12 is close to the first antenna 116 of the neuromodulation device 100. In some embodiments, the second antenna 12 is carried by a flexible carrier 9 that is configured to be positioned on or sufficiently near the sleeping surface while the patient sleeps to maintain the position of the first antenna 116 within the target volume of the electromagnetic field generated by the second antenna 12. Through this approach, the system 10 can deliver therapy to improve SDB (such as OSA), for example, by stimulating the HGN through a shorter, less invasive procedure. The elimination of an on-board, implanted power source in favor of an inductive power scheme can eliminate the need for batteries and the associated battery changes over the patient's life.

In some embodiments, the system 10 can include one or more sensors (not shown), which may be implanted and/or external. For example, the system 10 can include one or more sensors carried by (and implanted with) the neuromodulation device 100. Such sensors can be disposed at any location along the lead 102 and/or electronics package 108. In some embodiments, one, some, or all of the conductive elements 114 can be used for both sensing and stimulation. Use of a single structure or element as the sensor and the stimulating electrode reduces the invasive nature of the surgical procedure associated with implanting the system, while also reducing the number of foreign bodies introduced into a patient. In certain embodiments, at least one of the conductive elements 114 is dedicated to sensing only.

In addition to or instead of inclusion of one or more sensors on the neuromodulation device 100, the system 10 can include one or more sensors separate from the neuromodulation device 100. In some embodiments, one or more of such sensors are wired to the neuromodulation device 100 but implanted at a different location than the neuromodulation device 100. In some embodiments, the system 10 includes one or more sensors that are configured to be wirelessly coupled to the neuromodulation device 100 and/or an external computing device (e.g., control unit 30, user interface 40, etc.). Such sensors can be implanted at the same or different location as the neuromodulation device 100, or may be disposed on the patient's skin.

The one or more sensors can be configured to record and/or detect physiological data (e.g., data originating from the patient's body) over time including changes therein. The physiological data can be used to select certain stimulation parameters and/or adjust one or more stimulation parameters during therapy. Physiological data can include an electromyography (EMG) signal, temperature, movement, body position, electroencephalography (EEG), air flow, audio data, heart rate, pulse oximetry, eye motion, and/or combinations thereof. In some embodiments, the physiological data can be used to detect and/or anticipate other physiological parameters. For example, the one or more sensors can be configured to sense an EMG signal which can be used to detect and/or anticipate physiological events such as phasic contraction of anterior lingual musculature (such as phasic genioglossus muscle contraction) and measure physiological data such as underlying tonic activity of anterior lingual musculature (such as tonic activity of the genioglossus muscle). Phasic contraction of the genioglossus muscle can be indicative of inspiration, particularly the phasic activity that is layered within the underlying tonic tone of the genioglossus muscle. Changes in physiological data include changes in one or more parameters of a measured signal (e.g., frequency, amplitude, spike rate, etc.), start and end of phasic contraction of anterior lingual musculature (such as phasic genioglossus muscle contraction), changes in underlying tonic activity of anterior lingual musculature (such as changes in tonic activity of the genioglossus muscle), and combinations thereof. In particular, changes in phasic activity of the genioglossus muscle can indicate a respiration or inspiration change and can be used to trigger stimulation. Such physiological data and changes therein can be identified in signals recorded from sensors during different phases of respiration including inspiration. As such, the one or more sensors can include EMG sensors. The one or more sensors can also include, for example, wireless or tethered sensors that measure, body temperature, movement (e.g., an accelerometer), breath sounds (e.g., audio sensors), heart rate, pulse oximetry, eye motion, etc.

In operation, the physiological data provided by the one or more sensors enables closed-loop operation of the neuromodulation device 100. For example, the sensed EMG responses from the genioglossus muscle can enable closed-loop operation of the neuromodulation device 100 while eliminating the need for a chest lead to sense respiration. Operating in closed-loop, the neuromodulation device 100 can maintain stimulation synchronized with respiration, for example, while preserving the ability to detect and account for momentary obstruction. The neuromodulation device 100 can also detect and respond to snoring, for example.

The system 10 can be configured to provide open-loop control and/or closed-loop stimulation to configure parameters for stimulation. In other words, with respect to closed-loop stimulation, the system 10 can be configured to track the patient's respiration (such as each breath of the patient) and stimulation can be applied during or prior to onset of inspiration, for example. However, with respect to open-loop stimulation, stimulation can be applying without tracking specific physiological data, such as respiration or inspiration. However, even under such an “open loop” scenario, the system 10 can still adjust stimulation and record data, to act on such information. For example, one way the system 10 can act upon such information is that the system 10 can configure parameters for stimulation to apply stimulation in an open loop fashion but can monitor the patient's respiration to know when to revert to applying stimulation on a breath to breath, close-loop fashion such that the system 10 is always working in a closed-loop algorithm to assess data. Treatment parameters of the system may be automatically adjusted in response to the physiological data. The physiological data can be stored over time and examined to change the treatment parameters; for example, the treatment data can be examined in real time to make a real time change to the treatment parameters. In some embodiments, the treatment parameters can be learned from the physiological data stored over time and used to adjust the therapy in real time. This learning can be patient-specific and/or across multiple patients.

Operating in real-time, the neuromodulation device 100 can record data (e.g., via one or more sensors) related to the stimulation session including, for example, stimulation settings, EMG responses, respiration, sleep state including different stages of REM and non-REM sleep, etc. For example, changes in phasic and tonic EMG activity of the genioglossus muscle during inspiration can serve as a trigger for stimulation or changes in stimulation can be made based on changes in phasic and tonic EMG activity of the genioglossus muscle during inspiration or during different sleep states. This recorded data can be uploaded to the user interface 40 and to the remote computing device(s) 80. Also, the patient can be queried to use the interface 40 to log data regarding their perceived quality of sleep, which can also be uploaded to the remote computing device(s) 80. Offline, the remote computing device(s) 80 can execute a software application to evaluate the recorded data to determine whether settings and control parameters can be adjusted to further optimize the stimulation therapy. The software application can, for example, include artificial intelligence (AI) models that, learn from recorded therapy sessions, how certain adjustments affect the therapeutic outcome for the patient. In this manner, through AI learning, the model can provide patient-specific optimized therapy.

### III. Neuromodulation Devices

FIGS. 2B-2D illustrate various views of an example configuration of the neuromodulation device 100. While specific features of the neuromodulation device 100 are discussed with reference to FIGS. 2B-2D, other configurations of the neuromodulation device 100 are possible. Example configurations of neuromodulation devices 100 within the scope of the present technology include the neuromodulation devices found in U.S. Provisional Patent Application No. 63/377,969, filed Sep. 30, 2022, U.S. patent application Ser. No. 16/865,541, filed May 4, 2020, U.S. patent application Ser. No. 16/866,488, filed May 4, 2020, U.S. patent application Ser. No. 16/866,523, filed May 4, 2020, and U.S. patent application Ser. No. 16/865,668, filed May 4, 2020. As previously mentioned, the device 100 can be configured to be implanted at a treatment site within submental and sublingual regions of the patient's head and deliver electrical energy at the treatment site to stimulate the HGN and/or one or more tongue protruser muscles (e.g., the genioglossus, the geniohyoid, etc.). The device 100 can include an electronics package 108 and a lead 102 coupled to and extending away from the electronics package 108. The lead 102 can comprise a lead body 104 having a plurality of conductive elements 114 and an extension portion 106 extending between the lead body 104 and the electronics package 108. The extension portion 106 can have a proximal end portion 106a coupled to the electronics package 108 via a first connector 110 and a distal end portion 106b coupled to the lead body 104 via a second connector 112.

The electronics package 108 can be configured to supply electrical current to the conductive elements 114 (e.g., to stimulate) and/or receive electrical energy from the conductive elements 114 (e.g., to sense physiological data). The extension portion 106 of the lead 102 can mechanically and/or electrically couple the electronics package 108 to the lead body 104. The extension portion 106 can comprise a polymeric material such as, but not limited to, a thermoplastic elastomer, a thermoplastic polyurethane, a silicone, or other suitable materials. The extension portion 106 can be sufficiently flexible such that it can bend so as to position the lead body 104 on top of, but spaced apart from, the electronics package 108. As discussed in greater detail below with reference to FIGS. 3A-3F, the neuromodulation device 100 is configured to be implanted within both a submental region and a sublingual region such that the electronics package 108 and lead body 104 are vertically stacked with one or more muscle and/or other tissue layers positioned therebetween. The flexibility of the extension portion 106 enables such a configuration.

In some embodiments, the extension portion 106 comprises a sidewall defining a lumen extending through the extension portion 106. The conductive elements 114 can be electrically coupled to the first antenna 116 and/or the electronics component 118 via one or more electrical connections extending through the lumen of the extension portion 106. For example, the proximal end portions of the electrical connections can be routed through the first connector 110 to the electronics component 118 on the electronics package 108. The electrical connections may comprise, for example, one or more wires, cables, traces, vias, and others extending through the extension portion 106 and lead body 104. The electrical connections can comprise a conductive material such as silver, copper, etc., and each electrical connection can be insulated along all or a portion of its length. In some embodiments, the device 100 includes a separate electrical connection for each conductive element 114. For example, in those embodiments in which the device 100 comprises eight conductive elements 114 (and other embodiments), the device 100 can comprise eight electrical connections, each extending through the lumen of the extension portion 106 from a proximal end at the electronics component 118 to a distal end at one of the conductive elements 114.

In some embodiments, the electronics component 118 comprise an application-specific integrated circuit (ASIC), a discrete electronic component, and/or an electrical connector. In these and other embodiments, the electronics component 118 can comprise, for example, processing and memory components (e.g., microcomputers, microprocessors, computers-on-a-chip, etc.), charge storage and/or delivery components (e.g., batteries, capacitors, electrical conductors) for receiving, accumulating, and/or delivering electrical energy, switching components (e.g., solid state, pulse-width modulation, etc.) for selection and/or control of the conductive elements 114. In some embodiments, the electronics component 118 comprise a data communications unit for communicating with an external device (such as external system 15) via a communication standard such as, but not limited to, near-field communication (NFC), infrared wireless, Bluetooth, ZigBee, Wi-Fi, inductive coupling, capacitive coupling, or any other suitable wireless communication standard. In some examples, the electronics component 118 include one or more processors having one or more computing components configured to control energy delivery via the conductive elements 114 and/or process energy and/or data received by the conductive elements 114 according to instructions stored in the memory. The memory may be a tangible, non-transitory computer-readable medium configured to store instructions executable by the one or more processors. For instance, the memory may be data storage that can be loaded with one or more of the software components executable by the one or more processors to achieve certain functions. In some examples, the functions may involve causing the conductive elements 114 to obtain data characterizing activity of a patient's muscles. In another example, the functions may involve processing data to determine one or more parameters of the data (e.g., a change in muscle activity, etc.). According to various embodiments, the electronics component 118 can comprise a wireless charging unit for providing power to other electronics component 118 of the device 100 and/or recharging a battery of the device 100 (if included).

The electronics package 108 can also be configured to wirelessly receive energy from a power source to power the neuromodulation device 100. In some embodiments, the electronics package 108 comprises a first antenna 116 configured to wirelessly communicate with the external system 15. As shown in FIG. 2B, in some embodiments the electronics component 118 can be disposed in an opening at a central portion of the first antenna 116. In other embodiments, the electronics component 118 and antenna 116 may have other configurations and arrangements.

The second antenna 12 can be configured to emit an electromagnetic field to induce an electrical current in the first antenna 116, which can then be supplied to the electronics component 118 and/or conductive elements 114. In some embodiments, the first antenna 116 comprises a coil or multiple coils. For example, the first antenna 116 can comprise one or more coils disposed on a flexible substrate. The substrate can comprise a single substrate or multiple substrates secured to one another via adhesive materials. For instance, in some embodiments the substrate comprises multiple layers of a heat resistant polymer (such as polyimide) with adhesive material between adjacent layers. Whether comprising a single layer or multiple layers, the substrate can have one or more vias extending partially or completely through a thickness of the substrate, and one or more electrical connectors can extend through the vias to electrically couple certain electronic components of the electronics package 108, such as the first antenna 116 and/or the previously discussed electronics component 118.

In some embodiments, the first antenna 116 comprises multiple coils. For example, the first antenna 116 can comprise a first coil at a first side of the substrate and a second coil at a second side of the substrate. This configuration can be susceptible to power losses due to substrate losses and parasitic capacitance between the multiple coils and between the individual coil turns. Substrate losses occur due to eddy currents in the substrate due to the non-zero resistance of the substrate material. Parasitic capacitance occurs when these adjacent components are at different voltages, creating an electric field that results in a stored charge. All circuit elements possess this internal capacitance, which can cause their behavior to depart from that of “ideal” circuit elements.

Advantageously, in some embodiments the first antenna 116 comprises a two-layer, pancake style coil configuration in which the top and bottom coils are configured in parallel. As a result, the coils can generate an equal or substantially equal induced voltage potential when subjected to an electromagnetic field. This can help to equalize the voltage of the coils during use, and has been shown to significantly reduce the parasitic capacitance of the first antenna 116. In this parallel coil configuration, the top and bottom coils are shorted together within each turn. This design has been found to retain the benefit of lower series resistance in a two-coil design while, at the same time, greatly reducing the parasitic capacitance and producing a high maximum power output. Additional details regarding the two-coil configuration can be found in U.S. application Ser. No. 16/866,523, filed May 4, 2020, which is incorporated by reference herein in its entirety.

The first antenna 116 (or one or more portions thereof) can be flexible such that the first antenna 116 is able to conform at least partially to the patient's anatomy once implanted. In some embodiments, the first antenna 116 comprises an outer coating configured to encase and/or support the first antenna 116. The coating can comprise a biocompatible material such as, but not limited to, epoxy, urethane, silicone, or other biocompatible polymers. In some embodiments, the coating comprises multiple layers of distinct materials.

With continued reference to FIGS. 2B-2D, the lead body 104 can comprise a substrate carrying one or more conductive elements 114 configured to deliver and/or receive electrical energy. In some embodiments, the lead body 104 (or one or more portions thereof) comprises flexible tubing with a sidewall defining a lumen. The lead body 104 can comprise a polymeric material such as, but not limited to, a thermoplastic elastomer, a thermoplastic polyurethane, a silicone, or other suitable materials. The lead body 104 can comprise the same material as the extension portion 106 or a different material. The lead body 104 can comprise the same material as the extension portion 106 but with a different durometer. In some embodiments, the lead body 104 has a lower durometer than the extension portion 106, which can enhance patient comfort.

As shown in FIGS. 2B-2D, the lead body 104 has a branched shape comprising a first arm 122 and a second arm 124. To facilitate this configuration, for example, the second connector 112 can be bifurcated and/or branching. The first arm 122 and the second arm 124 can each extend distally and laterally from the second connector 112 and/or the distal end portion 106b of the extension portion 106. The first arm 122 can comprise a proximal portion 122a, a distal portion 122b, and an intermediate portion 122c extending between the proximal portion 112a and the distal portion 122b. Similarly, the second arm 124 can comprise a proximal portion 124a, a distal portion 124b, and an intermediate portion 124c extending between the proximal portion 124a and the distal portion 124b. In some embodiments, the first arm 122 can comprise a cantilevered, free distal end 123 and/or the second arm 124 can comprise a cantilevered, free distal end 125. The first arm 122 and/or the second arm 124 can include one or more fixation elements 130, for example the fixation elements 130 shown at the distal end portions 122b, 124b of the first and second arms 122, 124 in FIGS. 2B-2D. The fixation elements 130 can be configured to securely, and optionally releasably, engage patient tissue to prevent or limit movement of the lead body 104 relative to the tissue.

While being flexible, the lead 102 and/or one or more portions thereof (e.g., the lead body 104, the extension portion 106, etc.) can also be configured to maintain a desired shape. This feature can, for example, be facilitated by electrical conductors that electrically connect the conductive elements 114 carried by the lead body 104 to the electronics package 108, by an additional internal shape-maintaining (e.g., a metal, a shape memory alloy, etc.) support structure (not shown), by shape setting the substrate comprising the lead 102, etc. In any case, one or more portions of the lead 102 can have a physical property (e.g., ductility, elasticity, etc.) that enable the lead 102 to be manipulated into a desired shape or maintain a preset shape. Additionally or alternatively, the lead 102 and/or one or more portions thereof (e.g., the lead body 104, the extension portion 106, etc.) can be sufficiently flexible to at least partially conform to a patient's anatomy once implanted and/or to enhance patient comfort.

The conductive elements 114 can be carried by the sidewall of the lead body 104. For example, the conductive elements 114 can be positioned on an outer surface of the sidewall and/or within a recessed portion of the sidewall. In some embodiments, one or more of the conductive elements 114 is positioned on an outer surface of the sidewall and extends at least partially around a circumference of the sidewall. The lumen of the lead body 104 can carry one or more electrical conductors that extend through the lumen of the lead body 104 and the lumen of the extension portion 106 from the conductive elements 114 to the electronics package 108. The sidewall can define one or more apertures through which an electrical connector can extend.

Each of the conductive elements 114 may comprise an electrode, an exposed portion of a conductive material, a printed conductive material, and other suitable forms. In some embodiments, one or more of the conductive elements 114 comprises a ring electrode. The conductive elements 114 can be crimped, welded, adhered to, or positioned over an outer surface and/or recessed portion of the lead body 104. Additionally or alternatively, each of the conductive elements 114 can be welded, soldered, crimped, or otherwise electrically coupled to a corresponding electrical connector. In some embodiments, one or more of the conductive elements 114 comprises a flexible conductive material disposed on the lead body 104 via printing, thin film deposition, or other suitable techniques. Each one of the conductive elements 114 can comprise any suitable conductive material including, but not limited to, platinum, iridium, silver, gold, nickel, titanium, copper, combinations thereof, and/or others. For example, one or more of the conductive elements 114 can be a ring electrode comprising a platinum iridium alloy. In some embodiments, one or more of the conductive elements 114 comprises a coating configured to improve biocompatibility, conductivity, corrosion resistance, surface roughness, durability, or other parameter(s) of the conductive element 114. As but one example, one or more of the conductive elements 114 can comprise a coating of titanium and nitride.

In some embodiments, one or more conductive elements 114 has a length of about 1 mm. Additionally or alternatively, one or more conductive elements 114 can have a length of about 0.25 mm, about 0.5 mm, about 0.75 mm, about 1.25 mm, about 1.5 mm, about 1.75 mm, about 2 mm, about 2.25 mm, about 2.5 mm, about 2.75 mm, about 3 mm, about 3.25 mm, about 3.5 mm, about 3.75 mm, about 4 mm, about 4.25 mm, about 4.5 mm, about 4.75 mm, about 5 mm, about 6 mm, about 7 mm, about 8 mm, about 9 mm, about 10 mm, more than 10 mm, or less than 0.25 mm. In any case, adjacent conductive elements 114 carried by one of the first or second arms 122, 124 can be spaced apart along a length of the arm by about 0.25 mm, about 0.5 mm, about 0.75 mm, about 1 mm, about 1.25 mm, about 1.5 mm, about 1.75 mm, about 2 mm, about 2.25 mm, about 2.5 mm, about 2.75 mm, about 3 mm, about 3.25 mm, about 3.5 mm, about 3.75 mm, about 4 mm, about 4.25 mm, about 4.5 mm, about 4.75 mm, about 5 mm, about 6 mm, about 7 mm, about 8 mm, about 9 mm, about 10 mm, more than 10 mm, or less than 0.25 mm. The conductive elements 114 can have the same length or different lengths.

While the device 100 shown in FIGS. 2B-2D includes eight conductive elements 114 (four conductive elements 114 carried by the first arm 122 and four conductive elements 114 carried by the second arm 124), other numbers and configurations of conductive elements 114 are within the scope of the present technology. For example, the first arm 122 can carry the same number of conductive elements 114 as the second arm 124, or the first arm 122 can carry a different number of conductive elements 114 as the second arm 124. The first arm 122 and/or the second arm 124 can carry one conductive element 114, two conductive elements 114, three conductive elements 114, four conductive elements 114, five conductive elements 114, six conductive elements 114, seven conductive elements 114, eight conductive elements 114, nine conductive elements 114, ten conductive elements 114, or more than ten conductive elements 114. In some embodiments, one of the first arm 122 or the second arm 124 does not carry any conductive elements 114.

The conductive elements 114 can be configured for stimulation and/or sensing. Stimulating conductive elements 114 can be configured to deliver energy to an anatomical structure, such as, for example, a nerve or muscle. In some embodiments, the conductive elements 114 are configured to deliver energy to a hypoglossal nerve of a patient to increase the activity of the patient's tongue protrusor muscles. Sensing conductive elements 114 can be used obtain data characterizing a physiological activity of a patient (e.g., muscle activity, temperature, etc.). In some embodiments, the sensing conductive elements 114 are configured to detect electrical energy produced by a muscle of a patient to obtain EMG data characterizing an activity of the muscle. In some embodiments, the sensing conductive elements are configured to measure impedance across the conductive elements. As but one example, in some embodiments the conductive elements 114 are configured to deliver energy to a hypoglossal nerve of a patient to increase activity of the genioglossus and/or geniohyoid muscles, and obtain EMG data characterizing activity of the genioglossus muscle and/or the geniohyoid muscle of the patient. Still, the conductive elements 114 can be configured to deliver energy to and/or measure physiological electrical signals from other patient tissues.

The function that each of the conductive elements 114 is configured to perform (e.g., delivering energy to patient tissue, receiving energy from patient tissue, etc.) can be controlled by a processor of the electronics component 118 of the electronics package 108. In some embodiments, one or more of the conductive elements 114 is configured for only one of delivering energy to patient tissue or receiving energy from patient tissue. In various embodiments, one or more of the conductive elements 114 is configured for both delivering energy to patient tissue and receiving energy from patient tissue. In some embodiments, the functionality of a conductive element 114 can be based, at least in part, on an intended positioning of the device 100 within a patient and/or the position of the conductive element 114 on the lead body 104. One, some, or all of the conductive elements 114 can be positioned relative to patient tissue, such as nerves and/or muscles, so that it may be desirable for the conductive element(s) 114 to be able to both deliver energy to the patient tissue and receive energy from the patient tissue. Additionally or alternatively, some conductive elements 114 can have an intended position relative to specific patient tissues so that only delivery of stimulation energy is desired while other conductive elements 114 can have an intended position relative to specific patient tissues so that only receipt of sensing energy is desired. Advantageously, the configurations of the conductive elements 114 can be configured in software settings (which can be facilitated by electronics component 118 of the electronics package 108) so that the configurations of the conductive elements 114 are easily modifiable.

Whether configured for stimulating and/or sensing, each of the conductive elements 114 can be configured and used independently of the other conductive elements 114. Because of this, all or some of conductive elements 114, whichever is determined to be most effective for a particular implementation, can be utilized during the application of stimulation therapy. For example, one conductive element 114 of the first arm 122 can be used as a cathode while one conductive element 114 of the second arm 124 is used as an anode (or vice versa), two or more conductive elements 114 of the first arm 122 can be used (one as the cathode and one as the anode) without use of any conductive elements 114 of the second arm 124 (or vice versa), multiple pairs of conductive elements 114 of the first and second arms 122, 124 can be used, or any other suitable combination. The conductive element(s) 114 used for sensing and/or stimulation can be selected based on desired data to be collected and/or desired modulation of neural or muscle activity. For example, specific pairs of the conductive elements 114 can be used for creating an electric field tailored to stimulation of certain regions of the muscle and/or HGN that causes favorable changes in tongue position and/or pharyngeal dilation. Additionally or alternatively, conductive element(s) 114 that are positioned in contact with muscle tissue when the device 100 is implanted may be more favorable to use for EMG sensing than conductive element(s) 114 that are not positioned in contact with muscle tissue.

The lead body 104 can have a shape configured to facilitate delivery of electrical energy to a specific treatment location within a patient and/or detection of electrical energy from a sensing location within the patient. The conductive elements 114 carried by the first arm 122 can be configured to deliver electrical stimulation energy to one hypoglossal nerve (e.g., the right or the left hypoglossal nerve) of a patient and the conductive elements 114 carried by the second arm 124 can be configured to deliver electrical stimulation energy to the other hypoglossal nerve (e.g., the other of the right or the left hypoglossal nerve) of the patient.

Without being bound by theory, it is believed that increased activity of the tongue protrusor muscles during sleep reduces upper airway resistance and improves respiration. Thus, devices of the present technology are configured to deliver stimulation energy to motor nerves that control the tongue protrusors. In some embodiments, the device 100 is configured to deliver stimulation energy to the hypoglossal nerve to cause protrusion of the tongue. Additionally or alternatively, the device 100 can be configured to receive sensing energy produced by activity of one or more muscles of a patient (such as the genioglossus muscle), which can be used for closed-loop delivery of stimulation energy, evaluation of patient respiration, etc.

The device can be configured to be implanted at an anatomical region of a patient that is bound anteriorly and laterally by the patient's mandible, superiorly by the superior surface of the tongue, and inferiorly by the patient's platysma. Such an anatomical region can include, for example, a submental region and a sublingual region. The sublingual region is bound superiorly by the oral floor mucosa and inferiorly by the mylohyoid and includes the plane between the genioglossus muscle and the geniohyoid muscle. The submental region is bound superiorly by the mylohyoid and inferiorly by the platysma muscle. FIGS. 3A-3F depict various views of the device 100 implanted within a patient. As shown in FIGS. 3A-3F, the neuromodulation device 100 is configured to be positioned such that the electronics package 108 is disposed on or near the inferior surface of the mylohyoid in a submental region while the lead body 104 is positioned between the geniohyoid and genioglossus in a sublingual region with the arms 122, 124 disposed along the left and right hypoglossal nerves. The arms 122, 124 can be positioned such that the conductive elements 114 are disposed near the distal arborization of the hypoglossal nerves that innervate the genioglossus. In particular, the conductive elements 114 can be positioned proximate the portions of the distal arborization that innervate the horizontal fibers of the genioglossus while limiting and/or avoiding stimulation of the portions of the distal arborization of the hypoglossal nerve that activate retrusor muscles. When implanted, the extension portion 106 of the lead 102 can extend in an anterior direction away from the electronics package 108 (towards the mandible), then bend superiorly and extend through the geniohyoid muscle until bending back posteriorly and extending within a tissue plane between the geniohyoid and genioglossus muscles. In some embodiments, the extension portion 106 straddles the right and left geniohyoid muscles.

The electronics package 108 can be sufficiently flexible so that, once implanted, the electronics package 108 at least partially conforms to the curvature of the mylohyoid. Additionally or alternatively, the electronics package 108 can have a shape reflecting the curvature of the mylohyoid. In some embodiments, the electronics package 108 can comprise fixation elements (similar to fixation elements 130 or otherwise) that are configured to engage the mylohyoid (or other surrounding tissue) and prevent or limit motion of the electronics package 108 once implanted.

The lead body 104 can be configured to be positioned between the genioglossus and geniohyoid muscles of a patient so that the conductive elements 114 are positioned proximate the hypoglossal nerve. Although not shown in FIGS. 3A-3F, the hypoglossal nerve is located between the genioglossus and fascia and/or fat located between the genioglossus and the geniohyoid. In some embodiments, the lead body 104 is configured to be positioned at or just inferior to the fat between the hypoglossal nerve and the geniohyoid and thus is not positioned in direct contact with the hypoglossal nerve. In any case, once the device 100 is implanted, the lead body 104 can extend posteriorly away from the distal end portion 106b of the extension portion 106. The lead body 104 can then branch laterally such that the first arm 122 of the lead body 104 is positioned proximate one of the patient's hypoglossal nerves and the second arm 124 is positioned proximate the contralateral hypoglossal nerve. The fixation elements 130 can engage patient tissue (e.g., the fat underlying the hypoglossal nerves, etc.) to prevent or limit motion of the first and second arms 122, 124 relative to the patient tissue.

As best shown in FIG. 3C, the arms 122, 124 of the lead body 104 can bend out of the plane of the extension portion 106, in addition to extending laterally away from the extension portion 106, such that the arms 122, 124 outline a somewhat concave shape. Advantageously, this concave shape can accommodate the convex inferior surface of the genioglossus and still keep the arms 122, 124 positioned near distal arborization of the hypoglossal nerve.

In some embodiments, conductive elements 114 are selected for use that selectively activate the protrusor muscles of a patient. In these and other embodiments, the specific positioning of the first and second arms 122, 124 relative to specific branches of the hypoglossal nerves need not be identified prior to stimulation of desired portions of the nerve and/or muscle. For example, in embodiments in which the lead body 104 includes more than two conductive elements 114, the combination of conductive elements 114 that is used for treating a patient can be selected based on physiological responses to test stimulations. For example, stimulation energy can be delivered to the hypoglossal nerve(s) via multiple combinations of conductive elements 114 and a physiological response (e.g., EMG data, tongue position, pharyngeal opening size, etc.) and/or a functional outcome (e.g., Fatigue Severity Scale, Epworth Sleepiness Scale, etc.) can be evaluated for each combination. Based on the evaluation(s), the conductive elements 114 that are selected to deliver stimulation energy can be conductive elements 114 that are associated with favorable responses/outcomes.

IV. Methods for Analyzing Respiration and/or Sleep State

Various embodiments of the present technology relate to methods for analyzing respiration and/or sleep state of a patient based, at least in part, on physiological data characterizing a physiological parameter of the patient. In some embodiments, the physiological data comprises EMG data characterizing activity of an anterior lingual muscle of the patient, such as the genioglossus muscle and/or the geniohyoid muscle. The methods herein can include (1) predicting a respiratory cycle of the patient (e.g., the onset of inspiration), (2) detecting a disordered breathing event (e.g., apnea, hypopnea, and/or other event associated with a sleep disorder), (3) detecting the current sleep state of the patient (e.g., whether the patient is awake or in REM, N1, N2, or N3 sleep), and/or (4) detecting other relevant patient states (e.g., motion, patient position).

The methods described herein can be performed by any embodiment of the systems and devices described above in Sections II and III. Some or all of the steps of the methods described herein can be implemented as computer-readable instructions (e.g., program code) that are configured to be executed by one or more processors of a computing device, such as the electronics package 108 of the neuromodulation device 100, the control unit 30 of the external system 15, etc. Additionally, any of the methods described herein can be combined with each other.

In various embodiments, the methods for analyzing respiration disclosed herein can be used to coordinate timing of neuromodulation therapy with one or more portions of a patient's respiratory cycle, which can improve the efficacy of the therapy and/or improve the patient's experience and satisfaction with the therapy. For example, applying neuromodulation before or during the inspiratory portion of the respiratory cycle can reduce airway resistance during inspiration, which allows greater air flow into the lungs and increases the patient's blood oxygen. By coordinating neuromodulation with inspiration, the airway can be dilated and/or stabilized when air is being drawn into the lungs, such that initial respiratory disturbances (e.g., hypopneas, apneas) and/or recurrent disturbances can be reduced or prevented.

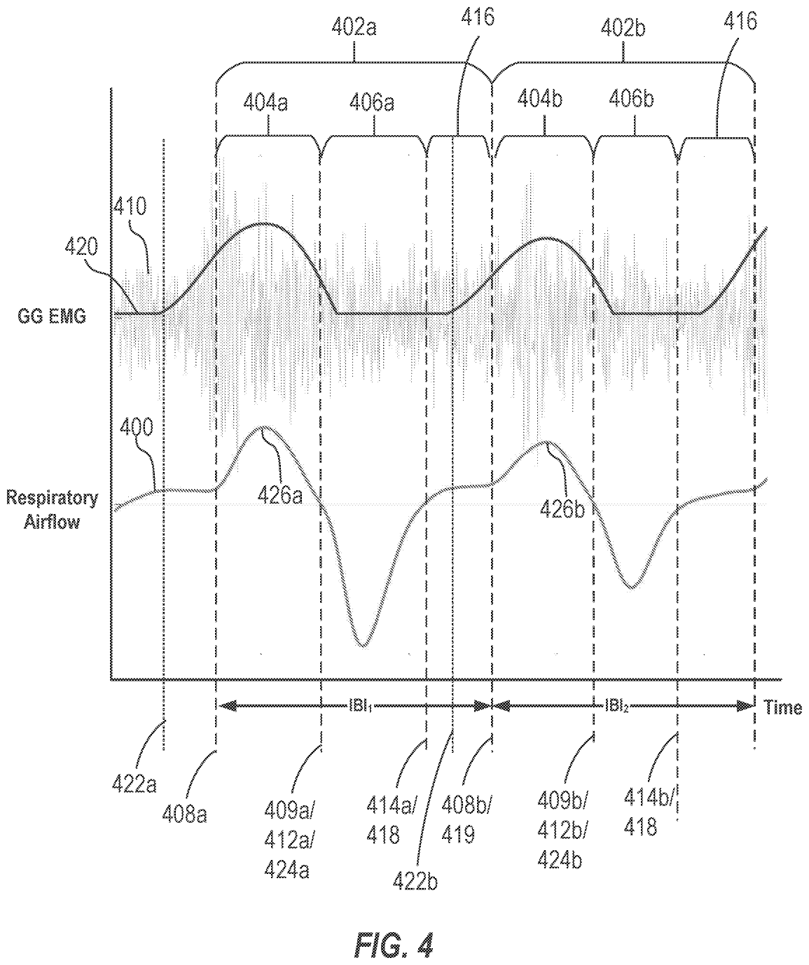

FIG. 4 is a graph illustrating an example of respiratory airflow data 400, genioglossus EMG (ggEMG) data 410, and an envelope of the ggEMG data (“ggEMG envelope 420”) over time during two consecutive respiratory cycles 402a, 402b. Respiration is the cyclical process of moving air into and out of the lungs. Each respiratory cycle 402a, 402b includes a respective inspiration 404a, 404b and a respective expiration 406a, 406b. Inspiration 404a, 404b is the portion of the respiratory cycle 402a, 402b during which external air travels into the patient's airway, and through the airway to the lungs. Contraction of inspiratory muscles, including the diaphragm and the external intercostal muscles, causes the volume of the thoracic cavity to increase, which in turn causes the pressure within the lungs to decrease. Air travels from a region of high pressure (e.g., external to the patient) to a region of low pressure (e.g., the lungs). As shown in FIG. 4, inspiration 404a, 404b occurs over a duration of time (e.g., a duration from about 1 second to about 2 seconds). Accordingly, each inspiration 404a, 404b can be characterized by a respective inspiration onset time 408a, 408b at which the inspiration 404a, 404b begins and a respective inspiration end time 409a, 409b at which the inspiration 404a, 404b ends.

Expiration 406a, 406b occurs after inspiration 404a, 404b and is the portion of the respiratory cycle 402a, 402b during which air travels away from the lungs and out of the body. Relaxation of the inspiratory muscles causes the volume of the thoracic cavity to decrease and causes pressure within the lungs to increase, thereby expelling air out of the lungs. As shown in FIG. 4, expiration 406a, 406b also occurs over a duration of time (e.g., a duration from about 1.5 seconds to about 2.5 seconds). Accordingly, each expiration 406a, 406b can be characterized by a respective expiration onset time 412a, 412b at which the expiration 406a, 406b begins and a respective expiration end time 414a, 414b at which the expiration 406a, 406b ends.

In some instances, a respiratory cycle 402a, 402b may include one or more lag periods during which little to no air moves into or out of the patient's body. For example, the expiration 406a, 406b of each respiratory cycle 402a, 402b may not begin immediately after the end of the corresponding inspiration 404a, 404b of the same respiratory cycle 402a, 402b, such that an inspiration lag (not shown in FIG. 4A) occurs between corresponding inspirations 404a, 404b and expirations 406a, 406b. The inspiration lag can be characterized by an inspiration lag onset time at which the inspiration lag begins and an inspiration lag end time at which inspiration lag ends. However, in many cases, the duration of the inspiration lag is very short (e.g., less than 100 milliseconds) or non-existent, and thus may be considered negligible for the respiratory analyses described herein.

As shown in FIG. 4, the inspiration 404b of the second respiratory cycle 402b may not begin immediately after the end of the expiration 406a of the first respiratory cycle 402a, such that an expiration lag 416 occurs between the expiration 406a and the inspiration 404b. The expiration lag 416 can be characterized by an expiration lag onset time 418 at which the expiration lag 416 begins and an expiration lag end time 419 at which expiration lag 416 ends. As shown in FIG. 4, the expiration lag onset time 418 can coincide with the expiration end 414a of the expiration 406a, and/or the expiration lag end time 419 can coincide with the inspiration onset 408b of the inspiration 404b. In some instances, the expiration lag 416 may have a non-negligible duration (e.g., a duration from about 100 milliseconds to about 2 seconds).

The total duration of a respiratory cycle 402a, 402b can be referred to herein as the “inter-breath interval.” In FIG. 4, IBI₁ refers to the inter-breath interval of the first respiratory cycle 402a, and IBI₂ refers to the inter-breath interval of the second respiratory cycle 402b. The inter-breath interval of a respiratory cycle 402a, 402b can be between two consecutive respiration markers denoting the same respiratory event. In the illustrated embodiment, for example, the inter-breath interval IBI₁ is measured between two consecutive inspiration onsets 408a, 408b. Alternatively or in combination, the inter-breath interval IBI₁ can be measured between two consecutive inspiration ends 409a, 409b, between two consecutive inspiration lag onsets, between two consecutive inspiration lag ends, between two consecutive expiration onsets 412a, 412b, between two consecutive expiration ends 414a, 414b, between two consecutive expiration lag onsets 418, and/or between two consecutive expiration lag ends 419. In some embodiments, the inter-breath interval IBI₁ can be measured between two consecutive inspiratory airflow peaks 426a, 426b. Typically, the inter-breath interval is between about 1 second and about 10 seconds, for example, between about 3 seconds and about 5 seconds. In some cases, the inter-breath interval can be greater in patients with sleep disordered breathing compared to patients with normal breathing. A respiratory rate can comprise a rate at which the respiratory cycle occurs. In some cases, the respiratory rate is the inverse of the inter-breath interval (e.g., about 6 breaths per minute to about 60 breaths per minute, for example about 12 breaths per minute to about 20 breaths per minute).

The distinct portions of a respiratory cycle can be correlated to other types of physiological data, such as EMG data of muscles involved in respiration. The timing and intensity of respiration is determined by the output of respiratory pattern generators in the medulla whose period and amplitude are modulated by a complex set of inputs from other brainstem regions with the overall goal of maintaining homeostasis and responding to physiological and behavioral demands. This periodic pattern, also referred to herein as the “respiratory drive,” is the input to the brainstem and spinal nuclei that control the muscles of inspiration in the diaphragm, thorax, and the upper airway, including the anterior lingual muscles (e.g., genioglossus, geniohyoid, styloglossus, hyoglossus, etc.). Accordingly, the timing of the respiratory drive can correlate directly to the activation of certain muscles involved in respiration, such that the activity of those muscles can be used to reliably detect and/or predict specific portions of the respiratory cycle (e.g., inspiration onsets 408a, 408b; expiration onsets 412a, 412b).

The ggEMG data 410 can characterize activity of the genioglossus muscle during the two consecutive respiratory cycles 402a, 402b. The ggEMG data 410 can represent summed activity from motor units of the genioglossus and can be measured by conductive elements of an implanted sensor (e.g., conductive elements 114 of neuromodulation device 100). The ggEMG data 410 can form a waveform representing the activity of the genioglossus over time. As shown in FIG. 4, the waveform can be an oscillating, zero-mean signal, such that the ggEMG envelope 420 generated from the waveform can be a useful representation of the overall trends in genioglossus activity for purposes of the respiratory analyses described herein.

The activity of the genioglossus muscle, as represented by the ggEMG data 410 and the ggEMG envelope 420, can be correlated to the timing of the respiratory cycles 402a, 402b. Specifically, tonic activity of the genioglossus can be present throughout the entirety of the respiratory cycles 402a, 402b (e.g., during both inspiration 404a, 404b and expiration 406a, 406b), while phasic activity may be present only or primarily during inspiration 404a, 404b. The tonic activity can be sustained activity of the genioglossus that is characterized by low-amplitude quiet periods correlating to the ggEMG envelope 420 being at a baseline level (e.g., approximately zero). The phasic activity can be transient activity of the genioglossus that is characterized by periodic, high-amplitude bursts correlating to the periodic peaks in the ggEMG envelope 420. The phasic activity can reflect the motor drive to the genioglossus muscle produced by the underlying respiratory drive to produce inspiration 404a, 404b. For example, prior to the onset of inspiration, the respiratory drive generates a motor drive to the genioglossus to activate and thereby dilate the airway in preparation for drawing air into the lungs. The motor drive can produce a characteristic increase in the phasic activity of the genioglossus at phasic onset times 422a, 422b before the inspiration onset times 408a, 408b, respectively. Accordingly, the ggEMG data 410 directly characterizes the times when the descending respiratory neural signals attempt to open the upper airway via activation of the genioglossus muscle. The phasic onset times 422a, 422b can correlate to the magnitude of the ggEMG envelope 420 rising above baseline and/or above a threshold level. Accordingly, the ggEMG data 410 and/or the ggEMG envelope 420 can be used to reliably detect and/or predict an upcoming inspiration 404a, 404b, as described further below. A duration between the phasic onset times 422a, 422b and the corresponding inspiration onset time 408a, 408b can be between about 1 microsecond and about 700 milliseconds.

As another example, at the end of inspiration and/or onset of expiration, the respiratory drive can reduce the motor drive to the genioglossus so that the genioglossus relaxes during expiration, when airway dilation is not required. This change in motor drive results in a characteristic decrease in phasic activity of the genioglossus at phasic end times 424a, 424b, which may occur before or concurrently with the expiration onset times 412a, 412b. The phasic end times 424a, 424b can correlate to the magnitude of the ggEMG envelope 420 falling below a threshold level and/or returning to the baseline level. Accordingly, the ggEMG data 410 and/or the ggEMG envelope 420 can also be used to reliably detect and/or predict an upcoming expiration 406a, 406b, as described further below.

Additionally, the activity of the genioglossus can also be used to determine other aspects of the patient's state, such as sleep state and/or changes in position. For example, the activation of genioglossus motor units can vary if a patient is awake or asleep, and/or if the patient is in REM sleep or non-REM sleep. The amplitudes of both phasic and tonic activity of the genioglossus, for example, can decrease during REM sleep relative to non-REM sleep. Specifically, the tonic activity of the genioglossus may significantly decrease and/or nearly cease during REM sleep. Phasic activity can still occur regularly during inspiration, but the amplitude of this phasic activity can be smaller than in non-REM sleep. Additionally, the respiratory rate of a patient varies with the patient's sleep state, which can be determined from the ggEMG envelope 420.

The underlying respiratory drive, and thus the motor drive to the genioglossus, remains consistent even when patient is experiencing OSA, such that the ggEMG data can still be used for respiratory analysis, sleep state analysis, etc., even if the patient's actual breathing is interrupted. In contrast, the activity of other muscles (e.g., intercostal muscles, diaphragm) may be disrupted by OSA and may therefore be unreliable for the analyses described herein. For example, respiratory drive to the diaphragm may increase when hyperpnea is induced in response to an obstructive respiratory event, while expansion of the chest and thorax during inspiration are reduced during obstructive events due to the smaller volume of air being drawn into the lungs.

Although certain embodiments herein are described with respect to the genioglossus and ggEMG data, this is not intended to be limiting, and the present technology can be used with other types of muscles whose activity is directly linked to the underlying respiratory drive and/or is not substantially affected by OSA. In some embodiments, the present technology can be used with one or more nasal muscles (e.g., compressor naris, dilator naris, alae nasi), one or more palatal muscles (e.g., palataoglossus, palatopharyngeus, levator veli palatini, tensor veli palatini, musculus uvulae), one or more tongue muscles (e.g., geniohyoid, hyoglossus, styloglossus, palatoglossus), one or more pharyngeal muscles (e.g., superior pharyngeal, middle pharyngeal, interior pharyngeal, stylopharyngeus), one or more mastication muscles (e.g., masseter, lateral pterygoids, medial pterygoids, temporalis), and/or one or more hyoid muscles, such as one or more suprahyoid muscles (e.g., mylohyoid, digastric), one or more infrahyoid muscles (e.g., sternohyoid, omohyoid, sternothyroid, thyrohyoid), and/or one or more laryngeal muscles (e.g., posterior cricoarytenoid, lateral cricoarytenoid, interarytenoid, thyroarytenoid, cricothyroid, aryepiglottic, thyroepiglottic).

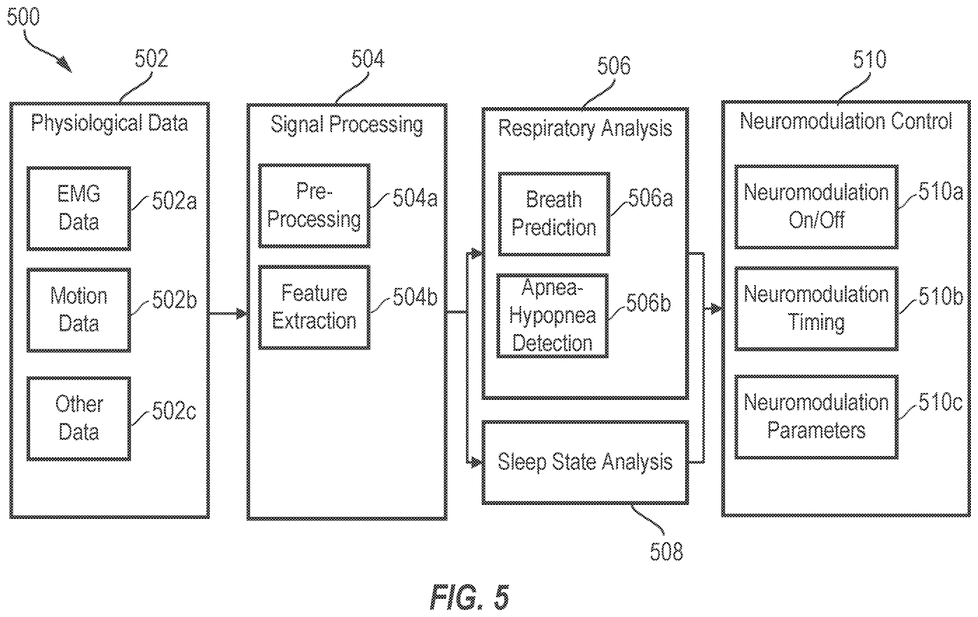

FIG. 5 is a flow diagram illustrating a general overview of an example workflow 500 for respiratory and sleep state analysis. As shown in FIG. 5, at block 502, the workflow 500 includes receiving physiological data of a patient, such as EMG data 502a, motion data 502b, and/or other suitable data 502c characterizing a physiological parameter of the patient. The physiological data can be obtained from a sensor of an implantable device, such as a neuromodulation device (e.g., neuromodulation device 100); an external device (e.g., external device 11); a wearable device (e.g., a smartwatch); or any other suitable device. Distinct types of physiological data can be obtained from a single sensor and/or from multiple, distinct sensors. Additionally or alternatively, a single type of physiological data can be obtained from a single sensor and/or from multiple, distinct sensors.

In some embodiments, the EMG data 502a comprises data characterizing electrical activity of an anterior lingual muscle, such as a genioglossus muscle, a geniohyoid muscle, and/or another suitable muscle of the tongue. For example, the EMG data 502a can comprise the ggEMG data 410 as shown in FIG. 4. The EMG data 502a can characterize voltages induced by the firing of motor units of the anterior lingual muscle, otherwise known as motor unit potentials (MUPs). As previously noted, the EMG data 502a can be obtained from an EMG sensor carried by an implantable device, an external device, a wearable device, etc. In some embodiments, the EMG data 502a is obtained from an EMG sensor carried by the neuromodulation devices of the present technology. For example, the conductive elements 114 of the neuromodulation device 100 can be positioned on the anterior lingual muscle of interest to record activity of the anterior lingual muscle. The EMG data 502a can be obtained from a surface EMG sensor configured to be positioned on a surface of a muscle of interest and/or from an intramuscular EMG sensor configured to be positioned within a muscle of interest. Such surface EMG sensors and/or intramuscular EMG sensors can be carried by an implantable neuromodulation device (e.g., neuromodulation device 100) and/or can be separate from such neuromodulation devices.

The motion data 502b can characterize position, orientation, and/or movement (e.g., displacement, velocity, acceleration, and/or vibration) of one or more portions of the patient's body, such as an anterior lingual muscle (e.g., the genioglossus, the geniohyoid), the head, the neck, the shoulders, the chest, the abdomen, and/or the back. Motion data 502b can be obtained from one or more motion sensors, such as accelerometers, gyroscopes, inertial measurement units (IMUs), etc. In some embodiments, the motion sensor(s) include an implanted motion sensor, such as a motion sensor carried by an implantable device (e.g., neuromodulation device 100). An implanted motion sensor can be positioned proximate to and/or on the anterior lingual muscle to generate motion data 502b thereof. In some embodiments, the motion sensor(s) include an external motion sensor, such as a motion sensor carried by an external device of the present technology (e.g., by the carrier 9 of the external device 11), etc.). An external motion sensor can be positioned proximate to and/or on the patient's head, chin, under-chin region, neck, shoulders, chest, abdomen, and/or back to generate motion data 502b thereof. Motion data 502b characterizing the position of the patient's head, for example, can provide useful information about the patient's sleep state (e.g., if the head is vertical, the patient is likely awake).

The other data 502c can include any data characterizing a physiological parameter of the patient that is useful for analyzing a patient's respiration, sleep state, motion, body position, and/or other states. Examples of such physiological parameters include, but are not limited to, patient sounds associated with physiological events (e.g., respiration, snoring, bruxism, apneas, swallowing, speech, movement, and/or others), pressure (e.g., airway pressure, pressure of the patient's body on a surface), heart rate, heart rate variability, body temperature, respiration rate, sleep patterns, etc. As another example, in some embodiments, the other data 502c can include electrooculography (EOG), as EOG measurements can be correlated to sleep state of the patient. For example, EOG measurements can be used to characterize eye movement. Rapid eye movement above a certain threshold can, for example, be correlated to a REM stage of sleep. EOG measurements can be obtained, for example, using one or more EOG sensors in a wearable such as an eye covering (e.g., eye mask, ski mask, goggles, etc.) having one or more EOG electrode sensors. Additionally or alternatively, in some embodiments other data 502c can include EMG data associated with other muscles not included in EMG data 502a. For example, other data 502c can include EMG measurements of one or more chin muscles to assess chin muscle tone for characterization of muscle atonia and/or other muscle disorders during a REM stage of sleep. Chin EMG measurements can be obtained, for example, using one or more EMG sensors in a wearable such as a chin strap, and/or an implanted device (e.g., chin implant).

In some embodiments, the other data 502c characterizes a parameter that is not physiological but is otherwise related to the state of the patient and/or the state of the neuromodulation systems disclosed herein. For example, the other data 502c can characterize a parameter related to the patient's environment, such as temperature, background, humidity of environment, light, etc. Such environmental parameters may affect the patient (e.g., the temperature of the sleeping environment may affect the sleep state of the patient) and thus, may be useful in analyzing the patient's state. As another example, the other data 502c can characterize the operation of a neuromodulation system (e.g., power delivered to an implantable device, stimulation energy delivered to the patient), which can influence the efficacy of a therapy implemented with the system and/or may provide useful information when analyzing the patient's state, such as detected sleep disordered breathing events.

The other data 502c can be obtained from any suitable sensor such as, but not limited to, a temperature sensor, a microphone or audio sensor (e.g., a condenser, a microelectromechanical (MEMS) transducer, a dynamic microphone, a carbon microphone, a ribbon microphone, a piezoelectric microphone), a heart rate sensor, a pulse oximetry sensor, an eye motion sensor, a ballistocardiograph sensor, a pressure sensor, a photoplethysmography sensor, a bedside monitor, a vibration sensor, a lighting sensor, a proximity sensor, a pneumatic sensor, a chest motion sensor, and others. The sensors for obtaining other data 502c can be include implanted sensors, external sensors, or suitable combinations thereof. For instance, an implanted sensor can be carried by an implantable device (e.g., neuromodulation device 100, implanted sensor(s)). An external sensor can be carried by an external system of the present technology (e.g., external system 15, etc.) or one or more portions thereof (e.g., carrier 9), a wearable device (e.g., a smart watch, a smart ring, a cardiac monitor, a chest motion monitor, eye covering, chin strap, etc.), disposed proximate to a patient (e.g., on or near the patient's bed), or can be directly attached to the patient's body.

At block 504, the workflow 500 can include processing the physiological data (e.g., “signal processing”). Signal processing can be performed to convert raw physiological data into a format suitable for use in subsequent analysis, and may comprise pre-processing the raw physiological data (block 504a), and/or extracting one or more features from the pre-processed physiological data and/or raw physiological data (block 504b). Pre-processing the physiological data (block 504a) can involve cleaning the data (e.g., by removing noise and/or artifacts from the data using filters or other suitable techniques), normalizing the data (e.g., by scaling the data, applying level limits to the data, rectifying the data), determining an envelope and/or other signal derived from the data, and/or otherwise placing the data into a better format for feature extraction and/or further analysis.

Feature extraction (block 504b) can involve determining one or more measurable values, properties, statistics, and/or transforms of the raw and/or pre-processed physiological data, which may then be used in analyzing the data. In some embodiments, some or all of the features correspond to a physiological event (e.g., a portion of a respiratory cycle, a change in sleep state, etc.). Feature extraction can include computing envelopes, statistics, and/or transformations using the physiological data. The resulting features can include, for example, estimates of amplitude, zero-crossing rate, peak positive value, moving mean, moving standard deviation, and/or frequency-domain features of the physiological data. In some embodiments, the resulting features can include, for example, a value of the data, a magnitude of the data, an amplitude of the data, a frequency of the data, a threshold crossing of the data, a complexity of the data, a range of the data, a variance of the data, a transform of the data, a statistical parameter of the data, or a temporal feature of the data. Some or all of the features can be computed at discrete time points, and/or some or all of the features can be computed over a given time window. The size of a time window over which features are extracted can vary depending on the type of analysis to be performed (e.g., shorter windows for respiratory analysis, longer windows for sleep state analysis, etc.). In some embodiments, the extracted features can be selected based on the type of physiological data being processed and/or an intended analysis to be performed with the features. For example, at least some of the features extracted for a respiratory analysis can differ from the features extracted for a sleep state analysis. Optionally, at least some of the features extracted for a respiratory analysis can be the same as the features extracted for a sleep state analysis.

The methods of processing the physiological data can vary based on the type of physiological data to be processed (e.g., the EMG data 502a can be processed differently from the motion data 502b and/or from other data 502c such as audio data). For example, the EMG data 502a can include a waveform, and the signal processing of block 504 can involve generating one or more envelopes of the waveform (also referred to herein as an “EMG envelope” or “breath envelope”). The envelope can be a smooth curve that outlines or generally follows the extremes of the waveform, and can be an upper envelope, a lower envelope, and/or any signal derived from the upper and/or lower envelopes. Generating the envelope can be an important step in embodiments where the raw EMG data 502a is a biphasic, oscillating, and/or zero-mean signal, since the presence of high frequency content in the raw data can make it difficult to perform certain types of analyses on the raw data. For instance, algorithms configured to detect events and/or trends (e.g., peak detection algorithms, rising/falling edge detection algorithms) may be ineffective due to the presence of multiple, high frequency peaks in the raw EMG data. In contrast, it may be significantly easier to determine trends and/or events in the envelope, since the high frequency content obscuring such trends and/or events may be reduced or removed altogether in the envelope.

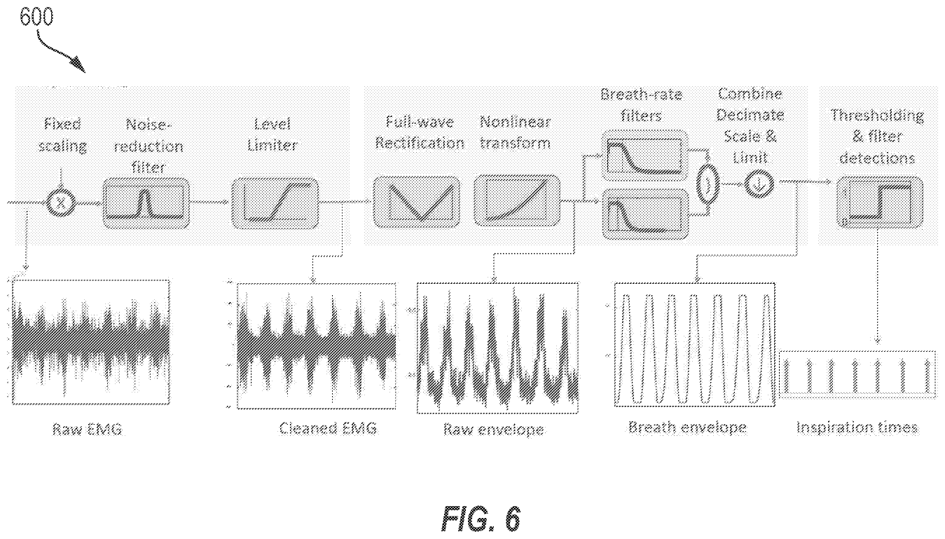

FIG. 6 illustrates an example workflow 600 for processing EMG data. The example workflow shown in FIG. 6 can be performed as part of the signal processing of block 504 of the workflow 500 shown in FIG. 5. The workflow 600 for processing EMG data includes receiving raw EMG data, which can be obtained by an implanted sensor, a sensor external to the patient, a wearable sensor, etc. In some embodiments, the raw EMG data is obtained from sensing conductive elements of an implantable neuromodulation device (e.g., conductive elements 114 of device 100, etc.). The raw EMG data can be received at a processor on an implantable device, on an external device, on a remote computing device, on the cloud, etc.

The raw EMG data can be processed to generate cleaned EMG data, which can have reduced noise and/or artifacts compared to the raw EMG data. Processing the raw EMG data can include fixed scaling (e.g., scaling and level normalization), noise-reduction filtering, level limiting, artifact detection and removal, and/or windowing. In some embodiments, processing the raw EMG data can comprise removing out-of-band noise (e.g., by filtering the raw EMG data, etc.), removing artifacts (e.g., removing a 60 Hz power line interference with a notch filter, etc.), and/or standardizing the raw EMG data with respect to a signal or noise level estimate (e.g., peak phasic amplitude, etc.). Knowledge of characteristics of interest of the EMG data can be used to suppress undesired characteristics of the raw EMG data. For example, the raw EMG data can be passed through a bandpass filter and rectified to remove unwanted signal frequencies that are not helpful for respiratory activity detection.

When processing the EMG data, one or more models of noise in the EMG data can be assumed. For example, an additive Gaussian noise model can be assumed so that a bandpass filter can be used with an empirically selected bandwidth that maximizes or enhances the respiration-synchronized modulation in the output. Such an approach is simple and effective and can be easily and efficiently implemented in hardware (e.g., ASIC/FPGA, etc.), and in computationally constrained environments. If the noise is additive and its spectral characteristics are known, a Wiener filter can be used instead of a bandpass filter, such that the noise spectrum can be determined using ground truth to select segments of noise-only EMG and compute the power spectrum of these segments. However, the additive noise assumption may be violated with EMG data. To address this problem, the EMG data can be modeled as a white noise process that is modulated (e.g., multiplied) by a quasi-periodic signal. Using a log-transformation, the signal can be modeled as a signal in additive noise, and a Wiener filter can be used. Another strategy is to use adaptive soft-thresholding of wavelet transform outputs to separate EMG signals into time-varying phasic and constant tonic components.

Raw EMG envelope data can be generated from the cleaned EMG data by applying full-wave rectification and nonlinear transformation to the cleaned EMG data. Full-wave rectification can involve determining the absolute value of the cleaned EMG data, thus allowing the data to be smoothed without losing useful information about the activity of the muscle. In some embodiments, cleaned EMG envelope data (e.g., a “breath envelope”) can be generated from the raw EMG envelope data by applying parallel or serial combinations of breath-rate filters, decimation, scaling, and/or limiting. The filtering, decimation, scaling, and/or limiting can be performed to remove or reduce noise and/or signal artifacts in the EMG data, and/or to enhance computational efficiency. For instance, when viewed in the frequency domain, the rectified raw EMG envelope data can contain low-frequency components that may correspond to variations in EMG power arising from changes in summed activity of phasic inspiratory motor units. Additionally or alternatively, the rectified raw EMG envelope data can contain high-frequency components that may correspond to the in-phase and out-of-phase summation of individual motor unit potentials, activity of non-phasic genioglossus units and motor units from neighboring muscles, and/or external electrical noise. In some embodiments, useful information for respiratory analysis is encoded in the low-frequency components with a bandwidth similar to respiratory rate (e.g., less than 1 Hz). Accordingly, the rectified raw EMG envelope data can be bandpass filtered based on the respiratory rate. Implementing such filters as a series or parallel combinations of smaller filter sections can enhance numerical stability and computational efficiency. The breath-rate filtered EMG envelope data may have a low bandwidth and can be accurately represented at a reduced sampling rate via decimation, which may allow for greater complexity in subsequent processing. The overall amplitude of the raw EMG envelope data including both signal and noise components may vary considerably across the measurements taken during a sleep session. Scaling the raw EMG envelope data by a local factor, which can be computed from short-term statistics of the EMG signal, can contain the excursion of the raw EMG envelope data over the typical respiratory timescale within an approximately uniform range over the recording duration. The measured EMG data can be corrupted by brief artifacts arising from patient movement and/or electrical interference. The amplitude of the EMG data generated by such artifacts can greatly exceed an amplitude of the envelope representing respiration. Moreover, in some embodiments, the timing of rise and fall of the envelope can be more important than the amplitude of the envelope during inspiration. To reduce or minimize the effect of envelope amplitude, which can be susceptible to corruption by artifacts and may be less important for subsequent processing, the scaled EMG envelope data can be limited to a maximum value. The cleaned EMG envelope data can be identical or generally similar to the ggEMG envelope 420 of FIG. 4.

The workflow 600 can comprise detecting one or more respiration markers, such as inspiration times, from the cleaned EMG envelope data. For example, detection of inspiration onsets can be performed by evaluating whether the magnitude of the cleaned EMG envelope data exceeds one or more thresholds (e.g., fixed thresholds, adaptive thresholds, and/or a combination of fixed and adaptive thresholds). Optionally, the detected inspiration onsets can be filtered to reduce false positives. The detected respiration markers can be used in further analyses and/or to control a neuromodulation therapy, as described further below.

Referring again to block 504 of FIG. 5, the features extracted from the EMG data 502a can be EMG signal properties that reflect motor drive to the genioglossus muscle. The features can include time-domain features, statistical features, temporal features, and/or time-frequency-domain features. The time-domain features can comprise a measure of the EMG envelope, which may approximate the motor unit firing rate (e.g., the level of activation of the muscle). Examples of such time-domain features include, but are not limited to, moving window averages of the absolute value, squared magnitude, RMS value, and/or Hilbert transform instantaneous magnitudes of the EMG envelope. In some embodiments, the time-domain features comprise a measure of the EMG waveform complexity, which may be influenced by the motor unit firing rate. Examples of such time-domain features include, but are not limited to, waveform length, zero-crossing rate, turns (e.g., number of slope changes) in a fixed window, mean amplitude between turns, and/or instantaneous frequency.

Statistical features of the EMG data 502a can overcome some limitations of conventional signal processing approaches due to the unique nature of the EMG data 502a. EMG data 502a may violate assumptions required for conventional signal processing approaches (e.g., motor unit spikes are quasi-periodic, rather than periodic; changes in motor drive recruit motor units that produce different waveforms). These issues can be mitigated through the use of statistical features. Examples of statistical features include, but are not limited to, direct measurements (e.g., sample magnitude histogram), order statistics of sample magnitude (e.g., quantiles, median), distribution tails (e.g., ratio of quantile to mean), and/or features characterizing departure from Gaussianity (e.g., skewness, kurtosis, Kolmogorov-Smirnov statistic, Lilliefors statistic).

Temporal features can reflect temporal dependencies in the EMG data 502a. In some embodiments, the EMG waveform is based on a combination of the spike sequence and the motor unit potential waveform, each of which has a temporal structure that may influence the resulting EMG waveform. Temporal features can be useful for filtering out additive noise and other interferences, for estimating spike rate and/or motor drive, and/or validating detections based on other features. Examples of temporal features include, but are not limited to, correlation measures such as magnitude autocorrelation (e.g., correlation coefficient vector, peak lags, principal components of an autocovariance matrix) and cepstral features (e.g., averages of the log-spectrum coefficients in the low- and high-bands, cepstral coefficients, warped-frequency cepstral coefficients).

Time-frequency domain features can provide a transform-domain representation of the frequency content of the EMG data 502a at one or more time points. Examples of time-frequency domain features include, but are not limited to, short-term Fourier Transformations, discrete wavelet coefficients, continuous wavelet coefficients, and/or frequency domain statistics. Some transforms, for example wavelets, capture components at multiple time scales, which can provide phasic information that permits timing recovery of periodic EMG components.

In some embodiments, one or more features can be extracted from the EMG waveform (e.g., the waveform represented in the raw EMG data and/or cleaned EMG data), such as one or more of the following: a value of the EMG waveform, a magnitude of the EMG waveform, an amplitude of the EMG waveform, a frequency of the EMG waveform, a threshold crossing of the EMG waveform, a complexity of the EMG waveform, a range of the EMG waveform, a variance of the EMG waveform, a transform of the EMG waveform, a statistical parameter of the EMG waveform, or a temporal feature of the EMG waveform. Alternatively or in combination, one or more features can be extracted from the envelope of the EMG waveform, such as one or more of the following: a value of the envelope, a magnitude of the envelope, an amplitude of the envelope, a frequency of the envelope, a threshold crossing of the envelope, a complexity of the envelope, a range of the envelope, a variance of the envelope, a transform of the envelope, a statistical parameter of the envelope, or a temporal feature of the envelope.

In some embodiments, signal processing of motion data 502b involves pre-processing the motion data 502b, then performing feature extraction on the pre-processed motion data. The pre-processing can comprise band-pass filtering the raw motion data to remove the effects of the gravity vector (e.g., if the sensor is an accelerometer, etc.) and/or the patient weight (e.g., if the sensor is a pressure sensor), sensor de-trending, and/or additional filtering using a combination of fixed linear filters, adaptive filters, and/or tracking algorithms (e.g., Kalman filters, etc.) to produce a vector time-series of measurements. These measurements can be segmented into overlapping windows, which can be approximately 60 seconds in length and/or up to 15 breath periods. Feature extraction can be performed on one, some, or all of these overlapping segments to produce feature vectors for determining information about one or more states of the patient (e.g., respiration, sleep, motion, body position.). For example, respiration cycle timing can be inferred from features such as direct motion measurements, zero-crossing rate, slope change rate, waveform length, mean amplitude between turns, envelope, and/or instantaneous frequency. As another example, the sleep state of the patient can be inferred from features that facilitate detecting large amplitude aperiodic motions, such as envelope fluctuation intensity, envelope fluctuation variance, and/or mean difference in cumulants at different lags.

In embodiments where the other data 502c includes audio data, the signal processing of the audio data can involve pre-processing the audio data, then performing feature extraction on the pre-processed audio data. Pre-processing of the audio data can comprise band-pass filtering and digitizing the raw audio data at an appropriate sampling rate. The digitized audio data can then be segmented into overlapping windows each having a predetermined duration (e.g., from about 10 milliseconds to about 40 milliseconds). Next, features can be extracted on each window to generate feature vectors for determining information about one or more states of the patient (e.g., respiration, sleep, motion, body position.). For example, respiration cycle timing can be inferred using extracted features such as temporal features, spectral features, combined time-frequency features, cepstral features, and/or DWT-based features. Additionally or alternatively, the audio data and/or the extracted features can be used to detect sleep events (e.g. snoring, swallowing) that may help qualify inferences made from other sensors (e.g., EMG sensors, accelerometers, etc.) regarding the patient's respiration and/or sleep state. For example, snoring detected prior to an inferred disordered breathing event may help validate the disordered breathing event.

Referring still to FIG. 5, the workflow 500 includes performing one or more analyses with the physiological data from block 502, the processed data from block 504, and/or the extracted features from block 504. For example, as shown in FIG. 5, the workflow 500 can include performing a respiratory analysis at block 506, which can include predicting the patient's respiratory cycle (“breath prediction,” block 506a) and/or detecting apneas, hypopneas, and/or other disordered breathing events (“apnea-hypopnea detection,” block 506b). In some embodiments, the respiratory analysis involves identifying and/or analyzing one or more portions of a respiratory cycle of the patient based on the physiological data and/or features extracted from the physiological data (e.g., the EMG data 502a and/or extracted features, the motion data 502b and/or extracted features, and/or the other data 502c and/or extracted features). For example, the respiratory analysis can involve identifying a respiration marker in the physiological data that correlates to a specific respiratory event (e.g., inspiration onset, expiration onset, apnea, etc.), and using the detected respiration markers and/or physiological data to determine the patient's respiration cycle, sleep state, and/or other states. The analysis can be performed using any suitable technique, including, but not limited to rule-based systems, machine learning models (e.g., decision trees, support vector machines, naive Bayes classifiers, k-nearest neighbor algorithms, logistic regression, self-organized maps, fuzzy logic systems, data fusion processes, time-series forecasting, artificial neural networks such as deep learning networks, ensemble methods), or suitable combinations thereof.

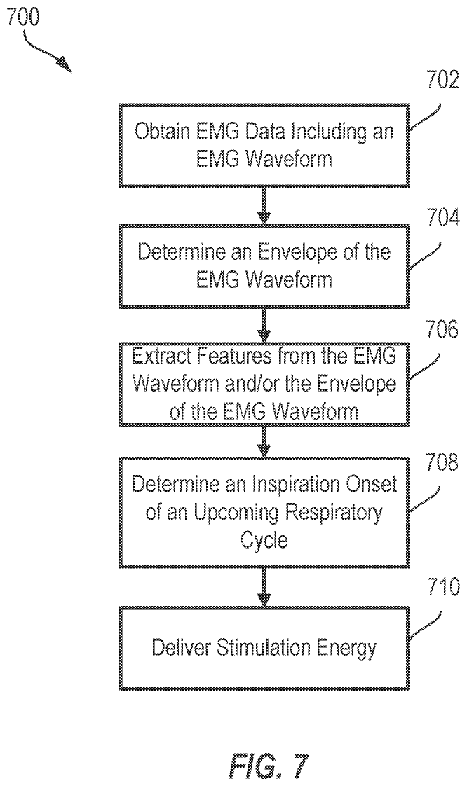

Respiratory analysis to predict the patient's respiratory cycle (block 506a) can comprise using the physiological data and/or extracted features to detect and/or predict one or more portions of a patient's respiratory cycle. In some embodiments, performing the respiratory analysis can comprise analyzing the EMG data to identify a characteristic pattern in the EMG data that correlates to a specific position of the respiratory cycle. For example, a characteristic increase in phasic activity in ggEMG data that correlates to the onset of inspiration can be identified and/or a characteristic decrease in phasic activity in ggEMG data that correlates to the onset of expiration can be identified, as previously described with respect to FIG. 4. In some embodiments, performing the respiratory analysis to predict a patient's breathing comprises predicting a time at which an upcoming portion of the respiratory cycle will occur based on one or more markers identified in the physiological data. In some embodiments, predictions of the patient's respiratory cycle are made based on physiological data and/or features extracted over a relatively short time window (e.g., a time window approximating the length of a single respiratory cycle, such as a time window from about 3 seconds to about 5 seconds in length), also referred to herein as “short-term inference.” Additional examples of methods for predicting a patient's respiratory cycle are described below in connection with FIGS. 7-11F.

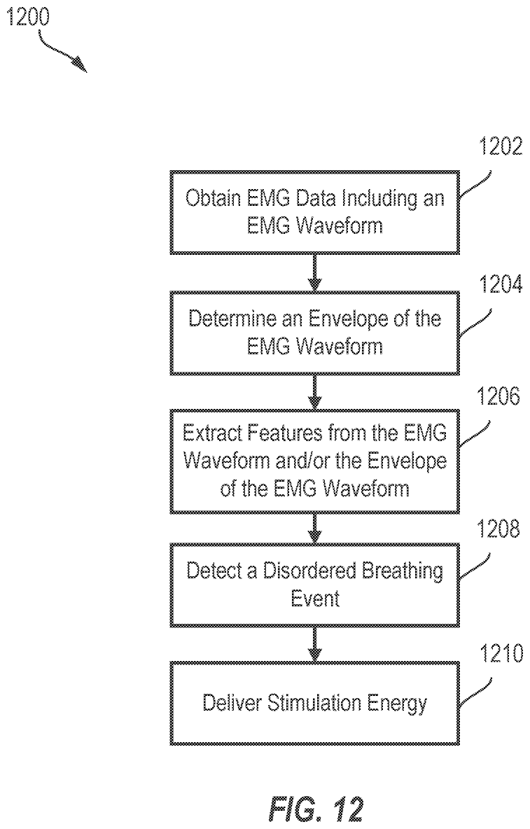

Respiratory analysis to detect an apnea, hypopnea, and/or other disordered breathing event (block 506b) can comprise using the physiological data and/or extracted features to identify one or more markers, characteristics, patterns, etc., within the data that correlate to the event. In some embodiments, detecting the disordered breathing event comprises determining whether the disordered breathing event occurred during a specific time window, what type of event occurred, and/or a characteristic of the event (e.g., severity, etc.). In some embodiments, detection of the disordered breathing event is performed using a combination of short-term inference and long-term inference. For example, short-term inference over several preceding respiratory cycles can be used to determine whether a disordered breathing event occurred during a present respiratory cycle. Additional examples of methods for detecting a disordered breathing event are described below in connection with FIG. 12.

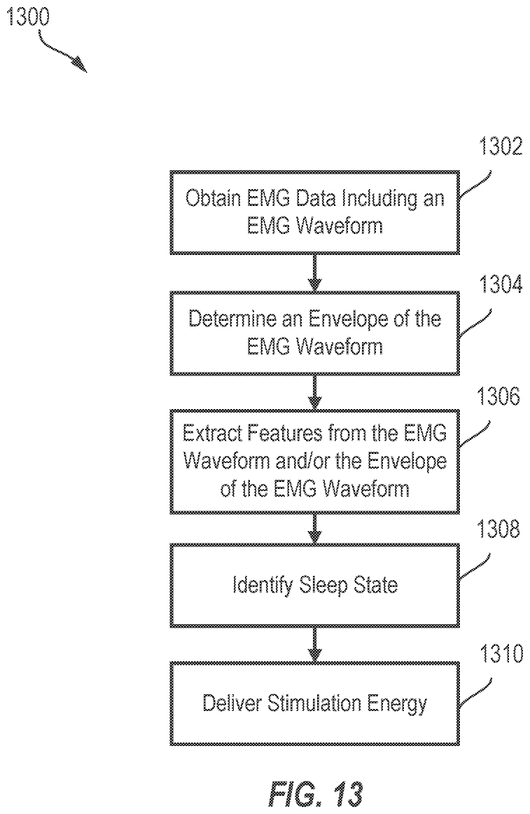

As shown in FIG. 5, the workflow 500 can include performing a sleep state analysis at block 508. The sleep state analysis can comprise identifying and/or analyzing one or more portions of a respiratory cycle of the patient based on the physiological data and/or extracted features to determine a current and/or past sleep state of the patient (e.g., awake, N1 sleep, N2 sleep, N3 sleep, REM sleep). Non-REM sleep comprises sleep without rapid eye movement and includes three stages: N1 sleep, N2 sleep, and N3 sleep. N1 sleep is a stage of light sleep during which the patient is transitioning from wakefulness to sleep and is easily arousable. During N1 sleep, a patient's heartbeat, breathing, and eye movements slow and muscles relax with occasional twitches. N1 sleep typically lasts several minutes in duration. N2 sleep, typically the longest of all the stages, is a period of light sleep before entering deeper sleep, where brain activity decreases, heartbeat and breathing slow, muscles relax even further, body temperature drops, and eye movement stops. N3 sleep is a stage of deep sleep during which the patient is not easily arousable. During N3 sleep, brain activity, muscle tone, pulse, and breathing rate further decrease as the body enters deeper relaxation. N3 sleep is believed to be critical to bodily restoration and recovery and growth, such that N3 sleep is required to feel refreshed when waking in the morning. During REM sleep, the eyes move rapidly, brain activity, heart rate, blood pressure, and breathing rate increase to levels approaching near wakefulness. During REM sleep, the body experiences atonia (temporary paralysis of the muscles), except for the eyes and the muscles that control breathing. REM sleep is believed to be essential to cognitive functions, such as memory, learning, and creativity. In some embodiments, determinations of the patient's sleep state are made based on physiological data and/or features extracted over a relatively long time window (e.g., a time window approximating the length of two, three, four, five, ten, or more respiratory cycles, such as a time window from about 30 seconds to about 60 seconds), also referred to herein as “long-term inference.” Additional examples of methods for determining a patient's sleep state are described below in connection with FIG. 13.

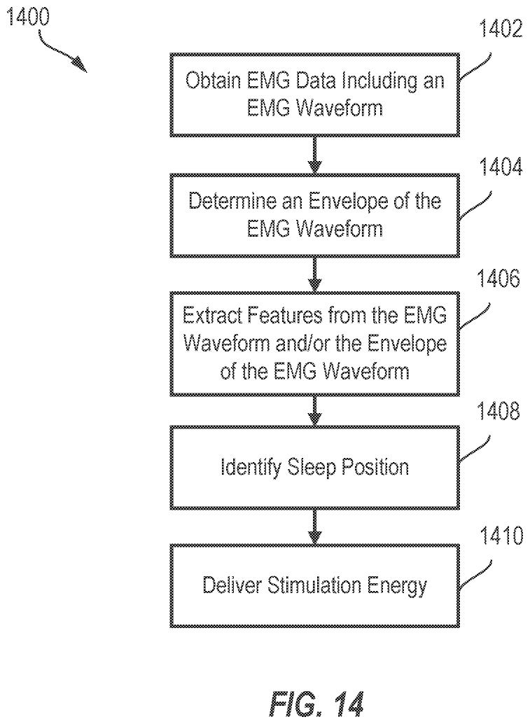

Although FIG. 5 illustrates two types of analyses that can be performed, respiratory analysis and sleep state analysis, the workflow 500 can alternatively or additionally include performing other types of analyses. For example, in some embodiments, the workflow 500 comprises performing a patient position analysis in which the physiological data and/or extracted features are analyzed to identify a position of a patient, such as whether the patient is sleeping in a prone, supine, or lateral position, for example. Additional examples of methods for determining a patient's position are described below in connection with FIG. 14.

Outputs of the analyses of blocks 506 and/or 508 can be used at block 510 for controlling neuromodulation delivered to a patient. The neuromodulation can comprise any of the neuromodulation therapies disclosed herein. For example, neuromodulation can be delivered to a hypoglossal nerve of a patient to modulate activity of an anterior lingual muscle of a patient to facilitate inspiration. In some embodiments, the neuromodulation therapy is delivered by an implantable device, such as implantable neuromodulation device 100. The neuromodulation therapy can be delivered by the same device carrying the sensor that collects the physiological data used for the analyses 506, 508, or the neuromodulation therapy can be delivered by a separate device. Additionally or alternatively, the neuromodulation therapy can be delivered by the same device that performs the analyses 506, 508 or the neuromodulation therapy can be delivered by a device that is separate from the device that performs the analyses 506, 508.

As shown in FIG. 5, the outputs of the analyses can be used to specify whether the neuromodulation should be on or off (block 510a). Delivering neuromodulation when the patient is awake can cause unnecessary discomfort in embodiments where the neuromodulation is intended to treat a sleep-related disorder. Accordingly, if the sleep state analysis of block 508 determines that the patient is awake, the neuromodulation can be turned off. In contrast, if the sleep state analysis of block 508 determines that the patient is asleep, the neuromodulation can be turned on.

In some embodiments, the outputs of the analyses of blocks 506 and/or 508 can be used at block 510b to determine neuromodulation timing. The neuromodulation timing can comprise a time at which the neuromodulation begins, a time at which the neuromodulation ends, a duration of the neuromodulation, etc. The neuromodulation timing can be coordinated with the timing of the patient's respiratory cycle determined in block 506a. It can be advantageous, for example, to begin delivering neuromodulation to the hypoglossal nerve to activate airway dilator muscles prior to the onset of inspiration. In other embodiments, neuromodulation can be delivered at a different time with respect to inspiration onset (e.g., concurrently with or after inspiration onset), or can be delivered relative to the timing of another portion of the respiratory cycle (e.g., expiration onset).

The outputs of the analyses of blocks 506 and/or 508 can be used to determine desired parameters of the neuromodulation (block 510c). Parameters of the neuromodulation can include parameters of stimulation energy to be delivered to the nerve and/or muscle, which can comprise, for example, amplitude, pulse width, duty cycle, polarity, frequency, ramp up time, ramp down time, ramp up rate, ramp down rate, and/or waveform. Respiratory analysis can be used to determine desired parameters of the neuromodulation to enhance an efficacy of the treatment. For example, if the output of the analysis at block 506b indicates that the patient is continuing to have hypopneas, apneas, and/or other disordered breathing events, the neuromodulation parameters can be modified to reduce the occurrence of these events. For example, the amplitude of the stimulation energy can be increased to further dilate the airway prior to and/or during inspiration. If the output of the analysis at block 506b indicates that fewer disordered breathing events occur after changing the neuromodulation parameters, the parameters can be held constant. However, if the output of the analysis at block 506b indicates that the same number or more disordered breathing events occur after changing the neuromodulation parameters, the parameters can be further optimized until a desirable outcome, such as fewer disordered breathing events, is achieved. In this manner, the disordered breathing detection analysis of block 506b can provide a measure of efficacy of a neuromodulation therapy, which can be used to assess the impact of changing parameters of the neuromodulation on the patient's disease.

Additionally or alternatively, the output of the sleep state analysis at block 508 can be used to determine desired parameters of the neuromodulation. For example, it may be beneficial to ramp up the intensity of the neuromodulation (e.g., increasing stimulation energy amplitude, increasing stimulation energy frequency, increasing stimulation energy pulse width, etc.) as the patient transitions from light sleep (e.g., N1 sleep, N2 sleep) into progressively deeper states of sleep (e.g., N3 sleep, REM sleep). Applying less intense neuromodulation during light sleep can prevent or limit arousing the patient and/or causing the patient discomfort with the stimulation, while applying more intense neuromodulation during deep sleep can enhance an efficacy of the neuromodulation. For example, during deeper sleep (e.g., N3 sleep, REM sleep), the tonic activity of the genioglossus can decrease compared to light sleep (e.g., N1 sleep, N2 sleep), such that that neuromodulation with higher intensity may be required and/or desired during deep sleep to achieve a similar level of airway dilation as compared to when the patient is in light sleep.

To enhance the efficacy of a neuromodulation therapy, it may be advantageous to apply the therapy during one or more specific portions of a patient's respiratory cycle. Neuromodulation can be used to activate airway dilator muscles and thereby cause the airway to open and/or prevent the airway from collapsing. This activation can be particularly useful during inspiration, when the negative pressure within the lungs rises and the patient attempts to draw air into the lungs. By applying neuromodulation to activate airway dilator muscles before and/or during inspiration, a greater volume of air can flow into the lungs during the inspiration.

It can also be useful to apply neuromodulation therapy prior to the onset of an inspiration to prevent an initial respiratory disturbance (e.g., a hypopnea or an apnea) that may lead to a cascade of recurrent respiratory disturbances. The respiratory system is controlled by physiological feedback loops, which may be disrupted in patients with OSA. For example, chemoreceptors detect when a patient's blood oxygen drops and/or when a patient's blood carbon dioxide increases after a hypopnea or an apnea. In response, the respiratory system induces hyperpnea (rapid and/or deep breathing) to attempt to rebalance the patient's blood gases. If the induced hyperpnea does not produce rebalancing of the blood gases (e.g., if the airway remains obstructed and another apnea or hypopnea occurs), the respiratory system may further increase the hyperpnea response. Patients with OSA may have a high-gain, unstable respiratory control system in which the respiratory system's corrective response (e.g., hyperpnea) is more significant than the disturbance prompting the corrective response (e.g., hypopnea, apnea, etc.). Accordingly, OSA patients may be particularly susceptible to recurrent hypopneas and apneas triggered by a single initial disturbance.

As a result, it can be advantageous to activate the airway dilator muscles prior to the onset of inspiration to prevent the initial respiratory disturbance from occurring at all. For example, neuromodulation can be delivered between about 1 microsecond and about 700 milliseconds prior to the onset of inspiration. In OSA patients, even a slight delay in initiating neuromodulation after the onset of inspiration occurs can trigger the cascade of recurrent disturbances, which can reduce an efficacy of the neuromodulation. In other embodiments, however, neuromodulation may be applied at the same time as the onset of inspiration, or after the onset of inspiration.

FIG. 7 is a flow diagram illustrating a method 700 for respiratory analysis. The method 700 begins at block 702 with obtaining EMG data including an EMG waveform. The EMG waveform can characterize activity of an anterior lingual muscle of the patient (e.g., the genioglossus). For example, the EMG data can be or include the ggEMG data 410 described above in connection with FIG. 4. The EMG data can be obtained using a sensor implanted in an anatomical region of the patient. The anatomical region can be a portion of the patient's head excluding the patient's neck, such as proximate to the patient's oral cavity and/or tongue. As but one example, the anatomical region can be bound anteriorly and laterally by the patient's mandible, superiorly by the superior surface of the tongue, and inferiorly by the patient's platysma. In some embodiments, the sensor is implanted in a sublingual region of the patient, a submental region of the patient, or a combination thereof. The sensor can be configured to be positioned adjacent to and/or in contact with the anterior lingual muscle. For example, the sensor can be configured to be positioned in a sublingual region in a plane between the genioglossus and the geniohyoid muscles such that the sensor is positioned in contact with the genioglossus and/or the geniohyoid. According to some embodiments, the EMG data can be obtained from the conductive elements 114 of the neuromodulation device 100 described with reference to FIGS. 2A-3F.

At block 704, the method 700 can include determining an envelope of the EMG waveform. The envelope of the EMG waveform can be a smooth curve that outlines or generally follows the extremes of the waveform to provide useful information about the overall change in amplitude of the EMG data over time. For example, the envelope can be or include the ggEMG envelope 420 of FIG. 4. The envelope can be determined in accordance with the methods described above in connection with FIG. 5 (e.g., at block 504) and FIG. 6.

At block 706, the method 700 can optionally include extracting one or more features from the EMG waveform and/or the envelope of the EMG waveform. As noted with reference to block 504b of FIG. 5, the features can comprise a measurable value, property, statistic, and/or transform of the EMG waveform and/or envelope. For example, the feature can comprise a measurable property of the EMG waveform and/or envelope representing a physiological event, such as onset of a certain portion of a respiratory cycle, a disordered breathing event, etc. In some embodiments, at least one feature is extracted from the EMG waveform. The EMG waveform can be the raw EMG waveform. Additionally or alternatively, the EMG waveform can be the cleaned EMG waveform, which can be obtained in accordance with the methods for generating cleaned EMG data described with reference to FIG. 6. The one or more features extracted at block 706 can comprise a value of the EMG waveform, a magnitude of the EMG waveform, an amplitude of the EMG waveform, a frequency of the EMG waveform, a threshold crossing of the EMG waveform, a complexity of the EMG waveform, a range of the EMG waveform, a variance of the EMG waveform, a transform of the EMG waveform, a statistical parameter of the EMG waveform, and/or a temporal feature of the EMG waveform. In some embodiments, at least one feature is extracted from the envelope of the EMG waveform. For example, the at least one feature can comprise a value of the envelope, a magnitude of the envelope, an amplitude of the envelope, a frequency of the envelope, a threshold crossing of the envelope, a complexity of the envelope, a range of the envelope, a variance of the envelope, a transform of the envelope, a statistical parameter of the envelope, and/or a temporal feature of the envelope. In other embodiments, however, block 706 is optional and may be omitted.

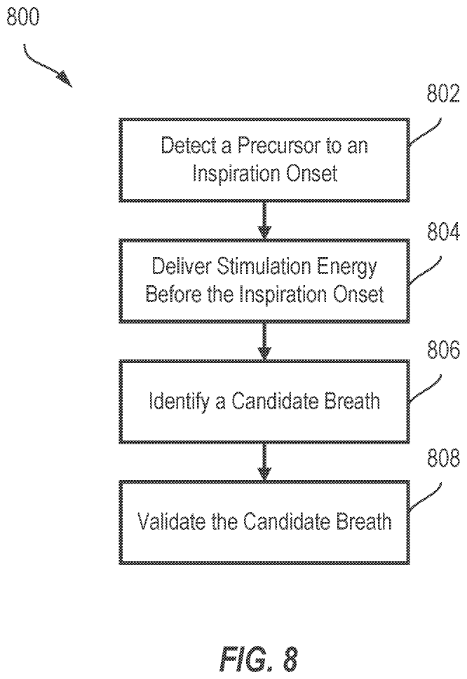

At block 708, the method 700 can include determining an inspiration onset of an upcoming respiratory cycle, based on the envelope of the EMG waveform and/or the extracted features. For example, the inspiration onset can be determined by detecting a precursor to the inspiration onset using a breath detection algorithm. The breath detection algorithm can analyze the envelope and/or extracted features to identify when the precursor occurs. The precursor can be characteristic muscle activity occurring before the inspiration onset, such as an increase in phasic activity of the genioglossus muscle that occurs prior to the onset of inspiration (e.g., at phasic onset times 422a, 422b of FIG. 4). The precursor can be detected according to a variety of methods. For example, the precursor can be detected based on whether the envelope exceeds a predetermined threshold. In another example, the precursor can be detected based on whether the rate of change of the envelope changes exceeds a predetermined threshold. In some embodiments, the features extracted at block 706, the EMG envelope, and/or the EMG waveform are input into a machine learning model that is trained to detect the precursor. Additional details of techniques for detecting the precursor to the inspiration onset are provided below in connection with FIG. 8.

As another example, the inspiration onset can be predicted using a breath prediction algorithm. The breath prediction algorithm can use previous EMG data of at least one previous respiratory cycle to predict the inspiration onset of an upcoming respiratory cycle. For instance, the breath prediction algorithm can determine time parameters from previous EMG data, and these time parameters can be used to calculate a predicted inspiration onset. As one specific example, the breath prediction algorithm can determine a time interval between the precursor to the inspiration onset and the inspiration onset and then can calculate a predicted inspiration onset of a future respiratory cycle based on a detected precursor to inspiration onset and the determined time interval. The breath prediction algorithm can be a rule-based algorithm, a machine learning model, or suitable combinations thereof. Additional details of techniques for predicting inspiration onset are provided below in connection with FIGS. 8-11F.

Optionally, the process of block 708 can include determining the inspiration onset using other types of data in addition to or instead of EMG data. Other types of data can include motion data as described with reference to block 502b of FIG. 5 and/or data characterizing a physiological parameter of the patient, a parameter related to a state of the patient, and/or a parameter related to a state of a neuromodulation system, as described with reference to block 502c of FIG. 5. The other data can be processed (e.g., via pre-processing and/or feature extraction) as previously described with respect to block 504 of FIG. 5. The breath prediction algorithm can predict an inspiration onset based on the other data and/or features extracted from the other data.

At block 710, the method 700 can continue with delivering stimulation energy. Stimulation energy can be delivered to a nerve and/or a muscle of a patient to modulate the activity of that nerve and/or muscle. For example, as described with reference to FIGS. 2A-3F, stimulation energy can be delivered to the hypoglossal nerve of the patient to increase activity of an anterior lingual muscle, such as the genioglossus and/or the geniohyoid, to reduce airway resistance and facilitate respiration. Stimulation energy can be delivered using the devices and systems of the present technology (e.g., neuromodulation system 10, neuromodulation device 100, etc.) and can be delivered in accordance with the neuromodulation methods disclosed herein with reference to FIGS. 2A-3F.

In some embodiments, stimulation energy is delivered before the determined onset of inspiration to dilate the airway and reduce airway resistance before the patient attempts to draw air into the airway. Stimulation energy can be delivered, for example, between about 1 microsecond and about 700 milliseconds prior to the determined onset of inspiration, between about 1 microsecond and about 300 milliseconds prior to the determined onset of inspiration, between about 1 microsecond and about 10 milliseconds prior to the determined onset of inspiration, between about 10 milliseconds and about 500 milliseconds prior to the determined onset of inspiration, or between about 100 milliseconds and about 300 milliseconds prior to the determined onset of inspiration. The stimulation energy can be delivered as soon as the precursor to inspiration onset is detected. Alternatively the stimulation energy can be delivered at a predetermined time after the precursor to inspiration onset has been detected. The predetermined time can be individualized for the patient or can be standardized across multiple patients. In some embodiments, the predetermined time is based on historical data for the patient, such as an average of previous durations between phasic activity onset time and inspiration onset times observed for the patient. Alternatively or in combination, the predetermined time can be based on historical population data, such as an average duration between phasic activity onset times and corresponding inspiration onset times observed for a population of patients. The predetermined time can be greater than or equal to about 1 microsecond, about 10 microseconds, about 100 microseconds, about 200 microseconds, about 300 microseconds, about 400 microseconds, about 500 microseconds, about 600 microseconds, about 700 microseconds, about 800 microseconds, about 900 microseconds, about 1 millisecond, about 5 milliseconds, about 10 milliseconds, about 100 milliseconds, about 200 milliseconds, about 300 milliseconds, about 400 milliseconds, about 500 milliseconds, about 600 milliseconds, or about 700 milliseconds. In some embodiments, stimulation energy can be delivered concurrently with inspiration onset or after inspiration onset.

FIG. 8 is a flow diagram illustrating a method 800 for respiratory analysis. The method 800 can be performed as part of the processes of blocks 708 and/or 710 of the method 700 of FIG. 7. The method 800 begins at block 802 with detecting a precursor to an inspiration onset. The inspiration onset can comprise a time at which air begins flowing into the airway and lungs, for example, as described with reference to inspiration onsets 408a, 408b as shown in FIG. 4. The precursor to inspiration onset can comprise characteristic muscle activity occurring before the inspiration onset, such as an increase in phasic activity of the genioglossus muscle that occurs prior to the onset of inspiration (e.g., at phasic onset times 422a, 422b) as described with reference to FIG. 4.

The precursor can be detected according to a variety of methods. For example, the precursor can be detected using a breath detection algorithm that analyzes the characteristics of an envelope of the EMG waveform. For example, the precursor can be detected by comparing the EMG envelope to a predetermined threshold. In some embodiments, an increase in the amplitude of the envelope of the ggEMG waveform (e.g., ggEMG envelope 420 of FIG. 4) past a predetermined threshold correlates to an increase in phasic activity of the genioglossus that is a precursor to inspiration. In some embodiments, the precursor can be detected when the magnitude of the EMG envelope exceeds a baseline level by a certain amount. The baseline level can be dynamically computed as the EMG envelope is evaluated. In some embodiments, the precursor can be detected by evaluating the rate of change of the EMG envelope. For instance, in the ggEMG envelope 420, a positive rate of change can represent increased recruitment of motor units in the genioglossus associated with phasic activity that occurs prior to inspiration.

Additionally or alternatively, the breath detection algorithm can comprise a machine learning model that is trained to detect the precursor, and the precursor can be detected by inputting features extracted from EMG data and/or the associated EMG envelope into the machine learning model. The machine learning model can comprise, but is not limited to, decision trees, support vector machines, naive Bayes classifiers, k-nearest neighbor algorithms, logistic regression, self-organized maps, fuzzy logic systems, data fusion processes, time-series forecasting, artificial neural networks such as deep learning networks, ensemble methods, or suitable combinations thereof. The machine learning model can be trained on data from the patient and/or data from other patients. The other patients can comprise a specific population of patients, such as patients with sleep disordered breathing, healthy patients, patients having similar characteristics as the patient being monitored (e.g., with respect to age, height, weight, body mass index, gender, diagnosis), etc. The output of the machine learning model can include whether or not the precursor was detected, a time stamp of the detected precursor, and/or a characteristic of the detected precursor.

Although the above techniques for detecting the precursor to inspiration onset are described in connection with EMG data, the process of block 802 can additionally or alternatively use motion data (e.g., as described with reference to block 502b of FIG. 5) and/or other data (e.g., as described with reference to block 502c of FIG. 5).

At block 804, the method 800 can include delivering stimulation energy before the inspiration onset, in response to detection of the precursor. Stimulation energy can be delivered immediately after detection of the precursor. For example, delivery of the stimulation energy can begin at a time corresponding to the phasic onset time 422a, immediately after the phasic onset time 422a, and/or after a predetermined time has elapsed following the phasic onset time 422a. Stimulation energy can be delivered in accordance with the methods described with reference to block 710 of FIG. 7.

At block 806, the method 800 can optionally include identifying a candidate breath. The candidate breath can be an event that is predicted to correlate to at least a portion of an actual respiratory cycle of the patient. The candidate breath can be identified in response to detection of a first respiration marker and a second, subsequent respiration marker. In some embodiments, the candidate breath corresponds to an entire respiratory cycle (e.g., including an inspiration and an expiration), such that the first and second respiration markers are two consecutive inspiration onsets, two consecutive inspiration ends, two consecutive expiration onsets, etc. Alternatively, the candidate breath can correspond to only a portion of a respiratory cycle. For example, the candidate breath can correspond to an inspiration only, such that the first respiration marker comprises an inspiration onset and/or a precursor to inspiration onset), and the second respiration marker comprises an inspiration end and/or an expiration onset.

In some embodiments, the first respiration marker is the precursor to inspiration onset detected in block 802, and the second respiration marker is an expiration onset. The expiration onset can be detected at block 806 based on the envelope of the EMG waveform. In some embodiments, the expiration onset can be detected by comparing the envelope to a predetermined threshold. For example, a decrease in the amplitude of the envelope of the ggEMG waveform (e.g., ggEMG envelope 420 of FIG. 4) below the predetermined threshold can correlate to a decrease in phasic activity of the genioglossus signaling the end of inspiration (which can coincide with the onset of expiration if the inspiration lag is negligible). The threshold for detecting expiration onset can be the same as or different than the threshold used to detect the precursor to inspiration onset. Alternatively or in combination, the expiration onset can be detected if the magnitude of the EMG waveform is sufficiently close to a baseline level. In a further example, the expiration onset can be detected by evaluating the rate of change of the EMG waveform. For instance, in the ggEMG envelope 420, a negative rate of change can represent decreased recruitment of motor units in the genioglossus associated with decreasing phasic activity at the end of inspiration.

In some embodiments, the expiration onset is detected by inputting features extracted from EMG data and/or the associated EMG envelope into a machine learning model that is trained to detect expiration onsets. Such a machine learning model can include, but is not limited to, decision trees, support vector machines, naive Bayes classifiers, k-nearest neighbor algorithms, logistic regression, self-organized maps, fuzzy logic systems, data fusion processes, time-series forecasting, artificial neural networks such as deep learning networks, and/or ensemble methods. The machine learning model can be trained on data obtained from the specific patient being treated and/or data obtained from other patients. The output of the machine learning model can be whether or not the expiration onset was detected and, optionally, a time stamp of when the expiration onset occurred.

Although the above techniques for detecting the expiration onset are described in connection with EMG data, the process of block 806 can additionally or alternatively use motion data (e.g., as described with reference to block 502b of FIG. 5) and/or other data (e.g., as described with reference to block 502c of FIG. 5).

Detection of a candidate breath can provide information about the accuracy of the precursor detection. If a candidate breath is detected (e.g., if both a precursor to inspiration onset and an expiration onset are detected in sequence), this may indicate a higher likelihood that inspiration actually occurred after detection of the precursor to inspiration onset. If a candidate breath is not detected (e.g., no expiration onset is detected after the precursor to inspiration onset), this may indicate that no actual inspiration occurred and that the detected precursor was incorrect. For example, the “precursor” may have been a signal artifact, or may have been caused by something other than breathing (e.g., patient movement, cardiac activity). Detection of a candidate breath, or lack thereof, can be used as feedback for improving the accuracy of the methods disclosed herein. For example, the breath detection algorithm used at block 802 for detecting a precursor to inspiration onset can be automatically updated based on whether a candidate breath was detected. As another example, the candidate breath information can be sent to a user (e.g., a healthcare professional), who can then manually update the breath detection algorithm. If a candidate breath was not detected, for example, one or more parameters of the breath detection algorithm can be modified to reduce the likelihood of the algorithm falsely detecting a precursor to inspiration onset.

At block 808, the method 800 can optionally include validating the candidate breath. The validation can be used to determine whether the candidate breath was an actual respiratory cycle (or portion thereof). The candidate breath can be validated based on an envelope of the EMG waveform (e.g., ggEMG envelope 420). For example, validating the candidate breath can include evaluating a shape of the EMG envelope, such as detecting the presence of a peak in the EMG envelope during the candidate breath time window. As shown in FIG. 4, the peaks in the ggEMG envelope 420 can correspond to periods of inspiration 404a, 404b in the respiratory cycles 402a, 402b, respectively. As another example, the EMG waveform, an envelope thereof, and/or one or more extracted features can be compared to one or more reference values (e.g., a predetermined threshold reference value).

In some embodiments, validating the candidate breath comprises inputting features extracted from the EMG envelope and/or the EMG waveform into a machine learning model that is trained to determine whether the candidate breath is an actual, valid breath. The features input into the machine learning model can be computed over the entire duration of the candidate breath and/or at specific time points in the candidate breath (e.g., inspiration onset, expiration onset). For example, the features can be computed for a portion of the candidate breath preceding the detected expiration onset so that the features characterize the inspiration portion of the candidate breath. In this example (and others), the features can characterize phasic activity of the genioglossus muscle that is known to have characteristic patterns during inspiration. The machine learning model used for validating the candidate breath can include, but is not limited to, decision trees, support vector machines, naive Bayes classifiers, k-nearest neighbor algorithms, logistic regression, self-organized maps, fuzzy logic systems, data fusion processes, time-series forecasting, artificial neural networks such as deep learning networks, and/or ensemble methods. The machine learning model can be trained on data obtained from the specific patient being treated and/or data obtained from other patients. The output of the machine learning model can comprise an indication of whether or not the candidate breath was a valid breath.

The outcome of the validity analysis at block 808 can be used to assess the accuracy of the precursor detection at block 802. If the candidate breath is determined to be valid, the detected precursor is more likely to be correct because the patient did in fact take a breath after the precursor was detected. If the candidate breath is determined to be invalid and no actual breath occurred, the detected precursor is more likely to be incorrect. For example, the “precursor” may have been a signal artifact or may have been caused by something other than breathing (e.g., patient movement, cardiac activity). Validation of the candidate breath, or lack thereof, can be used as feedback for improving the accuracy of the methods disclosed herein. For example, the breath detection algorithm of block 802 and/or the algorithm used at block 806 for identifying a candidate breath can be automatically updated based on whether or not the detected candidate breath was valid. Additionally or alternatively, the validated breath information can be sent to a user (e.g., healthcare professional) who can then manually update the algorithms used at blocks 802 and/or 806. If a candidate breath was not valid, for example, one or more parameters of the breath detection algorithm can be modified to reduce the likelihood of the algorithm falsely detecting a precursor to inspiration onset, and/or one or more parameters of the algorithm used at block 806 can be modified to reduce the likelihood of the algorithm falsely detecting a candidate breath.

The method 800 of FIG. 8 can be modified in many ways. For example, in some embodiments, the method 800 includes blocks 802 and 804, but blocks 806 and 808 are omitted (e.g., candidate breath detection and validation are not performed). In other examples, the method 800 can include blocks 802, 804, 806, but block 808 is omitted (e.g., validation is not performed). According to various embodiments, blocks 806 and 808 can be combined into a single process. Moreover, although FIG. 8 illustrates stimulation energy being delivered at block 804 prior to inspiration onset, the stimulation energy can be delivered at block 804 concurrently with or after inspiration onset. The method 800 can use other types of data besides EMG data to detect the precursor to inspiration onset, identify the candidate breath, and/or validate the candidate breath. For example, the method 800 can use motion data as described at block 502b of FIG. 5 and/or other data as described at block 502c of FIG. 5.

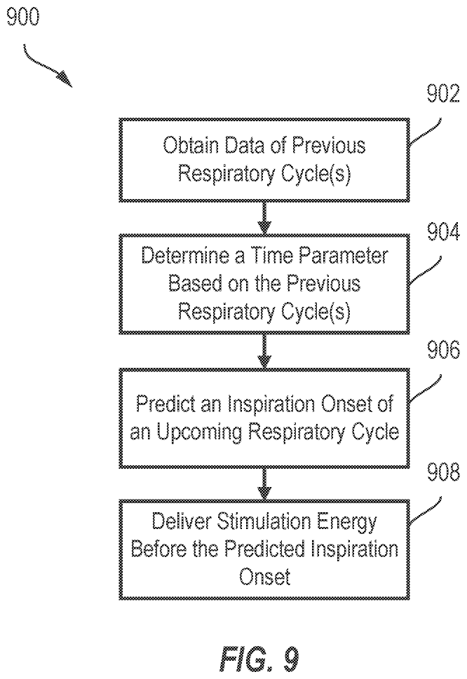

FIG. 9 is a flow diagram illustrating a method 900 for respiratory analysis. The method 900 begins at block 902 with obtaining data of at least one previous respiratory cycle of the patient. The data can be physiological data obtained during the at least one previous respiratory cycle, such as EMG data. The EMG data can include an EMG waveform and/or an envelope of the EMG waveform. For example, the data can comprise the ggEMG waveform 410 and/or the ggEMG envelope 420 as described with reference to FIG. 4, and/or the EMG data 502a described with reference to FIG. 5. Optionally, other types of data can be used, such as the motion data of block 502b of FIG. 5 and/or the other data of block 502c of FIG. 5. The data of the at least one respiratory cycle can include one or more features extracted from EMG data, motion data, and/or other data.

The at least one previous respiratory cycle can include one, two, three, four, five, ten, etc., previous respiratory cycles. The previous respiratory cycle(s) can be cycles immediately preceding the respiratory cycle for which the prediction is to be made. In some embodiments, the previous respiratory cycle(s) can be cycles occurring prior to the respiratory cycle immediately preceding the respiratory cycle for which the prediction is to be made. The previous respiratory cycle(s) can be from the same sleep period as the respiratory cycle for which the prediction is to be made, or can from a different sleep period (e.g., a different night). According to various embodiments, the data of the previous respiratory cycle(s) can include validated breath data generated by (i) detecting a candidate breath (including detecting the first and second respiration markers), e.g., as described above in connection with blocks 802 and 806 of the method 800, and (ii) validating the candidate breath, e.g., as described above in connection with block 808 of the method 800.

At block 904, the method 900 can include determining a time parameter based on the previous respiratory cycle(s) of the patient. The time parameter can characterize the timing and/or the duration of one or more portions of the previous respiratory cycle(s). For example, the time parameter can comprise an inspiration onset time, an inspiration end time, an inspiration duration, an inspiration lag onset time, an inspiration lag end time, an inspiration lag duration, an expiration onset time, an expiration end time, an expiration duration, an expiration lag onset time, and expiration lag end time, an expiration lag duration, and/or an inter-breath interval.

The time parameter can be calculated from physiological data (e.g., such as EMG data) of the previous respiratory cycle(s) and/or features extracted from the physiological data. Certain portions of the physiological data, for example, can correlate to respiration markers (e.g., precursor to inspiration onset, inspiration onset, expiration onset) of an individual respiratory cycle, and thus can be used to calculate timing for that respiratory cycle (e.g., inter-breath interval, inspiration duration, expiration duration), as previously described with respect to FIG. 4. The respiration markers can be detected by comparing the physiological data and/or feature(s) to a predetermined threshold, identifying a change in the data and/or feature(s), and/or identifying a pattern in the data and/or feature(s).

As one example, the time parameter can be an inter-breath interval corresponding to the total duration of a respiratory cycle (e.g., IBI₁ and IBI₂ in FIG. 4). The inter-breath interval can be calculated in various ways. For instance, the inter-breath interval can be the duration between two consecutive precursors to inspiration onsets, which can be detected from EMG data as described elsewhere herein. As another example, the inter-breath interval can be the duration between two consecutive inspiration onsets. If the time between the precursor to inspiration onset and the corresponding inspiration onset is assumed to be consistent, the timing of the precursor as detected from EMG data can be used to determine the timing of the corresponding inspiration onset. Additionally or alternatively, the inter-breath interval can be the duration between two consecutive expiration onsets, which can be detected from EMG data as described elsewhere herein.

According to various embodiments, the time parameter can be determined using a breath prediction algorithm. The breath prediction algorithm can be used to calculate the time parameter for each previous respiratory cycle. In some embodiments, the breath prediction algorithm can use the last measured value of the time parameter as the current value of the time parameter. Alternatively, the breath prediction algorithm can calculate a single time parameter from a plurality of time parameters from previous respiratory cycles based on an average value, minimum value, and/or maximum value of the individual time parameters (e.g., an average, minimum, and/or maximum inter-breath interval). In some embodiments, the breath prediction algorithm can calculate a weighted average of the time parameters, in which each of the individual time parameters is multiplied by a weighting factor to increase or decrease the relative contribution of the individual time parameter to the average time parameter. The weighting factor can be based on the timing of the respiratory cycle relative to the other respiratory cycles (e.g., more recent respiratory cycles can be weighted more than older respiratory cycles), a confidence level of the validity of the respiratory cycles (e.g., use lower weighting if there is low confidence that the respiratory cycle includes a valid breath), etc. In some embodiments, the time parameter can comprise a moving average of measured values of the time parameter over a fixed number of previous respiratory cycles. The breath prediction algorithm can compute a first order smoother comprising an average of measured values of the time parameter from a number of previous respiratory cycles computed over an exponential window.

Additionally or alternatively, the breath prediction algorithm can include a machine learning model trained to determine the time parameter from the previous respiratory cycle data. In some embodiments, determining the time parameter comprises extracting features from the previous respiratory cycle data (e.g., from the cleaned EMG data and/or the EMG envelope) and inputting the extracted features into the machine learning model. The extracted features can be any of the examples described herein. The machine learning model can include, but is not limited to, decision trees, support vector machines, naive Bayes classifiers, k-nearest neighbor algorithms, logistic regression, self-organized maps, fuzzy logic systems, data fusion processes, time-series forecasting, artificial neural networks such as deep learning networks, and/or ensemble methods. The machine learning model can be trained on data of the specific patient and/or data from other patients. The output of machine learning model can be the time parameter. In some embodiments, the machine learning model can predict an inter-breath interval for an upcoming respiratory cycle, which can be used to predict a corresponding inspiration onset of the upcoming respiratory cycle. Additionally or alternatively, the machine learning model can directly predict the inspiration onset of the upcoming respiratory cycle.

Optionally, the breath prediction algorithm can determine the time parameter based on other data besides data corresponding to the previous respiratory cycle(s) of the patient. For example, the breath prediction algorithm can determine the time parameter based on data from other patients. The other patients can comprise a specific population of patients, such as patients with sleep disordered breathing, healthy patients, patients having similar characteristics as the patient being monitored (e.g., with respect to age, height, weight, body mass index, gender, diagnosis), etc. In some embodiments, the breath prediction algorithm can calculate the time parameter based on other patient data (e.g., average inter-breath interval from a population of patients), combined with data for the specific patient (e.g., average inter-breath interval from historical respiratory cycles of the patient). In this example, and others, the breath prediction algorithm can calculate a weighted average using the average inter-breath interval from the population of patients and the average inter-breath interval from the patient's historical data and, optionally, may weight the patient's own historical data more heavily.

At block 906, the method 900 can include predicting an inspiration onset of an upcoming respiratory cycle, based on the determined time parameter. In some embodiments, the breath prediction algorithm can predict the inspiration onset by detecting a respiration marker of a current respiratory cycle, and then adding the determined time parameter to the time at which the detected respiration marker occurred to calculate the time at which the respiratory marker is predicted to occur in the next respiratory cycle. For example, the time parameter can be an inter-breath interval and the respiration marker can be an inspiration onset, such that the upcoming inspiration onset can be predicted by adding the inter-breath interval to the time at which the previous inspiration onset occurred. As another example, the time parameter can be an inter-breath interval and the respiration marker can be a precursor to inspiration onset, such that the upcoming precursor to inspiration onset can be predicted by adding the inter-breath interval to the time at which the previous precursor occurred. In such embodiments, the predicted precursor can serve as the prediction of the inspiration onset.

In some embodiments, the processes of blocks 904 and 906 can be combined. For example, the breath prediction algorithm can comprise a machine learning model that directly predicts the timing of an upcoming inspiration onset from the data of the previous respiratory cycles obtained at block 902. In such embodiments, the time parameter determined at block 904 by the machine learning model is the predicted inspiration onset.

At block 908, the method 900 can include delivering stimulation energy before the predicted inspiration onset. The stimulation energy can be delivered in accordance with the processes of block 710 of the method 700 shown in FIG. 7. For example, stimulation energy can be delivered to a hypoglossal nerve of a patient to increase activity in the genioglossus muscle and therefore dilate the airway prior to the onset of inspiration to enhance efficacy of a neuromodulation therapy. In some embodiments, a neuromodulation system of the present technology (e.g., neuromodulation system 10) can be programmed to deliver stimulation energy at predetermined time intervals. Such predetermined time intervals can be based on respiration data from a population of patients and/or from historical data of the patient to be treated. For example, the predetermined intervals can correspond to an average inter-breath interval of the patient and/or an average inter-breath interval of a population of patient to deliver the stimulation energy about as frequently as inspiration is expected to occur. The processes of block 908 can include altering such predetermined intervals so that the stimulation energy is delivered before the predicted inspiration onset of block 906. Altering the predetermined intervals can comprise altering a duration of one or more of the intervals, altering a start time of one or more of the intervals, altering an end time of one or more of the intervals, and/or altering a spacing between two or more of the intervals.

The method 900 can be modified in many ways. For example, as previously noted, blocks 904 and 906 can be combined, such that the determined time parameter is the predicted inspiration onset. In some embodiments, for example, as described with reference to block 710 of FIG. 7, the stimulation energy of block 908 can be delivered concurrently with or after inspiration onset. The method 900 can include additional steps, such as updating the breath prediction algorithm based on data from the patient and/or data from a population of patients. For example, the method 900 can include assessing whether the predicted inspiration onset time matched an actual inspiration onset time and updating the breath prediction algorithm based on the assessment. Such an assessment can comprise detecting a candidate breath and/or validating the candidate breath, for example, as described with reference to blocks 806 and 808 of FIG. 8.

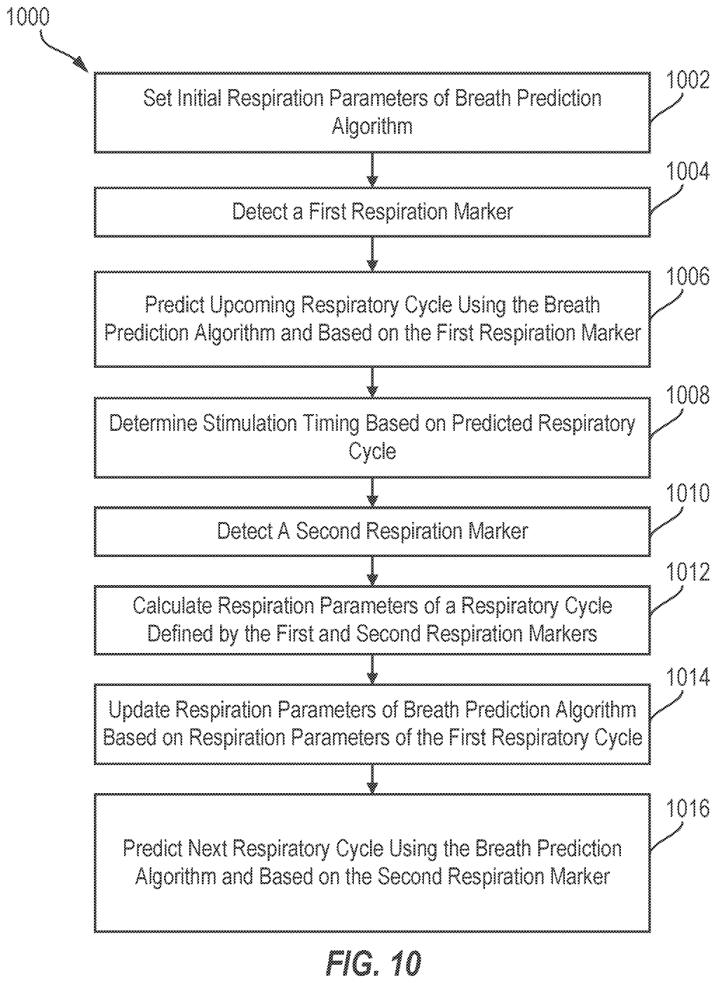

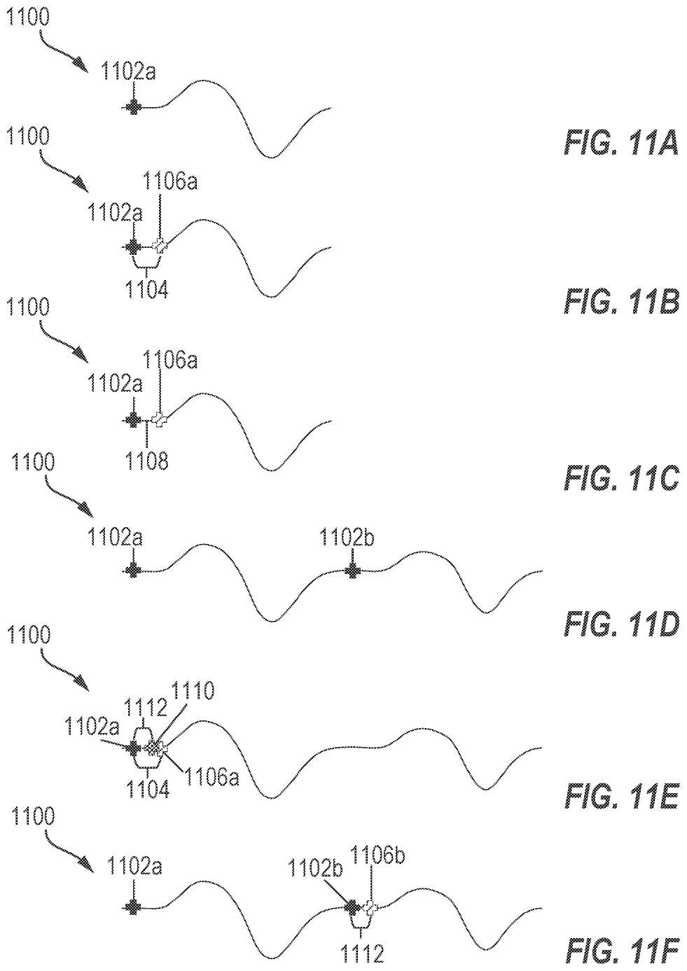

FIG. 10 is a flow diagram illustrating another method 1000 for respiratory analysis and FIGS. 11A-11F are graphs of respiratory airflow 1100 illustrating the respiratory analysis of the method 1000. The method 1000 can be performed as part of the processes of blocks 708 and/or 710 of the method 700 of FIG. 7. The method 1000 can begin at block 1002 with setting initial respiration parameters of a breath prediction algorithm. The initial respiration parameters can characterize one or more parameters of a respiratory cycle. For example, the initial respiration parameters can comprise time parameters associated with one or more respiratory cycles such as, but not limited to, an inspiration onset time, an inspiration end time, an inspiration duration, an inspiration lag onset time, an inspiration lag end time, an inspiration lag duration, an expiration onset time, an expiration end time, an expiration duration, an expiration lag onset time, an expiration lag end time, an expiration lag duration, or an inter-breath interval. In some embodiments, the time parameter characterizes an expected duration of time between two physiological events and/or two features. For example, the time parameter can characterize an expected duration of time between a characteristic change in phasic EMG activity of a genioglossus muscle serving as a precursor to inspiration onset, and the inspiration onset itself.

The initial respiration parameters can be based on prior data from the patient and/or data from a population of patients. For example, in embodiments where the initial respiration parameters include a time parameter (e.g., inter-breath interval, inspiration onset time, expiration onset time), the time parameter can be determined from physiological data (e.g., EMG data, motion data, other physiological data) of the patient and/or population of patients, in accordance with techniques described elsewhere herein. In some embodiments, the breath prediction algorithm includes and/or uses an average initial respiration parameter based on the prior data, an upper limit of the initial respiration parameter based on the prior data, and/or a lower limit of the initial respiration parameter based on the prior data.

At block 1004, the method 1000 can include detecting a first respiration marker characterizing one or more portions of a respiratory cycle. The first respiration marker can be, for example, an inspiration onset, an inspiration end, an inspiration lag onset, an inspiration lag end, an expiration onset, an expiration end, an expiration lag onset, an expiration lag end, a precursor to inspiration onset, a precursor to inspiration lag onset, a precursor to expiration onset, and/or a precursor to expiration lag onset. In the embodiment of FIG. 11A, for example, the first respiration marker is a precursor to inspiration onset 1102a. The first respiration marker can be detected based on physiological data of the patient, using any of the techniques described herein. For instance, the precursor to inspiration onset can be detected from EMG data (e.g., ggEMG data) by comparing an envelope of the EMG data to a predetermined threshold, evaluating a rate of change of the EMG envelope, and/or inputting the EMG envelope and/or features extracted from the EMG envelope into a machine learning model, in accordance with the methods of block 802 of FIG. 8.

At block 1006, the method 1000 can predict an upcoming respiratory cycle (or one or more portions thereof) using the breath prediction algorithm and based on the first respiration marker. For example, as shown in FIG. 11B, the breath prediction algorithm can predict an upcoming inspiration onset 1106a by adding an expected duration 1104 to the precursor to inspiration onset 1102a. The expected duration 1104 can be a duration between the precursor 1102a and the inspiration onset 1106a and can be based on previous respiratory data of the patient and/or a population of patients. If the first respiration marker comprises an inspiration onset, the breath prediction algorithm can add an inter-breath interval (e.g., the duration of a complete respiratory cycle) to the detected inspiration onset to predict an upcoming inspiration onset. If the first respiration marker comprises an inspiration end, the breath prediction algorithm can add an expiration duration (e.g., the duration of expiration and/or the duration of expiration lag) to the detected inspiration end to predict an upcoming inspiration onset.

At block 1008, the method 1000 can include determining when to apply stimulation based on the predicted respiratory cycle. As described herein, it can be advantageous to stimulate a nerve and/or a muscle of a patient during a specific portion of a respiratory cycle, such as prior to the onset of inspiration. As illustrated in FIG. 11C, stimulation 1108 can be initiated prior to the predicted inspiration onset 1106a. In some embodiments, the process of block 1008 involves delivering stimulation energy according to the determined timing. The stimulation energy can be delivered as previously described with respect to block 710 of the method 700 of FIG. 7.

At block 1010, the method 1000 includes detecting a second respiration marker. The second respiration marker can be, for example, an inspiration onset, an inspiration end, an inspiration lag onset, an inspiration lag end, an expiration onset, an expiration end, an expiration lag onset, an expiration lag end, a precursor to inspiration onset, a precursor to inspiration lag onset, a precursor to expiration onset, and/or a precursor to expiration lag onset. The second respiration marker can characterize the same or similar information as the first respiration marker but can occur subsequent to the first respiration marker. For example, as illustrated in FIG. 11D, the second respiration marker is the precursor to the next inspiration onset 1102b. Accordingly, detection of the second respiration marker can indicate that a complete respiratory cycle has occurred between the first and second respiration markers. In some embodiments, the second respiration marker characterizes a different portion of the respiratory cycle than the first respiration marker. For example, the first respiration marker can be a precursor to inspiration onset or an inspiration onset while the second respiration marker can be an inspiration end and/or an expiration onset. The second respiration marker can be detected based on physiological data of the patient, using any of the techniques described herein. For example, a second respiration marker comprising a precursor to inspiration onset or an expiration onset can be detected from EMG data, such as ggEMG data, in accordance with the methods of block 802 of FIG. 8.

At block 1012, the method 1000 can then calculate respiration parameters of a respiratory cycle defined by the first and second respiration markers. For example, as shown in FIG. 11E, the time of the actual inspiration onset 1110 may differ from the predicted time of inspiration onset 1106. Thus, the duration 1112 between the precursor 1102a and the actual inspiration onset 1110 can differ from the duration 1104 between the precursor 1102a and the predicted inspiration onset 1106a. In some embodiments, the method 1000 comprises calculating an actual inter-breath interval, an actual inspiration duration, an actual expiration duration, and/or an actual duration between a precursor to inspiration and inspiration onset for a detected respiratory cycle.

At block 1014, based on the calculated respiration parameters, the method 1000 can update the respiration parameters of the breath prediction algorithm. In some embodiments, updating the respiration parameters of the breath prediction algorithm comprises replacing one or more of the initial respiration parameters of the breath prediction algorithm with a corresponding calculated respiration parameter. Additionally or alternatively, updating a respiration parameter of the breath prediction algorithm can comprise replacing the parameter with an average, a median, a root mean square, or another suitable derivative of prior calculated values of the respiration parameter from the patient and/or from a population of patients. For example, as noted above, updating a respiration parameter can comprise determining a moving average and/or a first order smoother of previously calculated values of the respiration parameter. Additionally or alternatively, in embodiments where the breath prediction algorithm uses a machine learning model, updating a respiration parameter can comprise using a supervised learning approach with the calculated values of the respiration parameter to determine an updated respiration parameter value. Updating the respiration parameters of the breath prediction algorithm can improve the accuracy of the breath prediction algorithm.

At block 1016, the method 1000 can then predict the next respiratory cycle or one or more portions thereof using the updated breath prediction algorithm and based on the second respiration marker. As illustrated in FIG. 11F, in some examples, the breath prediction algorithm can use the duration 1112 between the precursor 1102a and actual inspiration onset 1110 of the first respiratory cycle to predict the inspiration onset 1106b for the next respiratory cycle. Specifically, the duration 1112 can be added to the precursor to inspiration onset 1102b of the next respiratory cycle to determine the predicted inspiration onset 1106.

FIG. 12 is a flow diagram illustrating an example method 1200 for detecting a disordered breathing event. The method 1200 can be performed as part of the apnea-hypopnea detection 506b of FIG. 5. The method 1200 can be used to assess whether a patient experienced a disordered breathing event during sleep, which can provide insight into the pathophysiology of the patient and/or efficacy of stimulation therapy, and/or can be used to control and/or modify stimulation parameters. The method 1200 can be combined with any of the other methods described herein (e.g., the method 700 of FIG. 7).

The method 1200 begins at block 1202 with obtaining EMG data including an EMG waveform. The EMG waveform can characterize activity of an anterior lingual muscle of the patient (e.g., the genioglossus). For example, the EMG data can be or include the ggEMG data 410 described above in connection with FIG. 4. The EMG data can be obtained using a sensor implanted in a sublingual region of a patient and/or a sensor positioned adjacent to and/or in contact with the anterior lingual muscle. According to some embodiments, the EMG data can be obtained from the conductive elements 114 of the neuromodulation device 100 described with reference to FIGS. 2A-3F.

At block 1204, the method 1200 can include determining an envelope of the EMG waveform. The envelope of the EMG waveform can be a smooth curve that outlines or generally follows the extremes of the waveform to provide useful information about the overall change in amplitude of the EMG data over time. For example, the envelope can be or include the ggEMG envelope 420 of FIG. 4. The envelope can be determined in accordance with the methods described above in connection with FIG. 5 (e.g., at block 504) and FIG. 6.

At block 1206, the method 1200 can optionally include extracting one or more features from the EMG waveform and/or the envelope of the EMG waveform. As noted with reference to block 504b of FIG. 5, a feature can comprise a measurable value, property, statistic, and/or transform of the EMG waveform and/or envelope. For example, the feature can comprise a measurable property of the EMG waveform and/or envelope representing a physiological event, such as onset of a certain portion of a respiratory cycle, a disordered breathing event, etc. In some embodiments, at least one feature is extracted from the EMG waveform. The EMG waveform can be the raw EMG waveform. Additionally or alternatively, the EMG waveform can be the cleaned EMG waveform, which can be obtained in accordance with the methods for generating cleaned EMG data described with reference to FIG. 6. The one or more features extracted at block 1206 can comprise a value of the EMG waveform, a magnitude of the EMG waveform, an amplitude of the EMG waveform, a frequency of the EMG waveform, a threshold crossing of the EMG waveform, a complexity of the EMG waveform, a range of the EMG waveform, a variance of the EMG waveform, a transform of the EMG waveform, a statistical parameter of the EMG waveform, and/or a temporal feature of the EMG waveform. In some embodiments, at least one feature is extracted from the envelope of the EMG waveform. For example, the at least one feature can comprise a value of the envelope, a magnitude of the envelope, an amplitude of the envelope, a frequency of the envelope, a threshold crossing of the envelope, a complexity of the envelope, a range of the envelope, a variance of the envelope, a transform of the envelope, a statistical parameter of the envelope, and/or a temporal feature of the envelope.

In some embodiments, the features of interest for the disordered breathing event detection can differ from the features of interest for the breath detection algorithm and/or the breath prediction algorithm. In other embodiments, the features of interest for the disordered breathing event detection can be the same as the features of interest for the breath detection algorithm and/or the breath prediction algorithm. In still other embodiments, however, block 1206 is optional and may be omitted.

At block 1208, the method 1200 can include detecting a disordered breathing event, based on the envelope of the EMG waveform and/or the extracted features. The disordered breathing event can be an apnea, a hypopnea, and/or another event associated with disordered respiration during sleep and can be detected using a disordered breathing event detection algorithm. The disordered breathing event detection algorithm can be configured to identify activity of the muscle that is indicative of the disordered breathing event. For example, during an apnea or a hypopnea, the amplitude of the phasic activity of the genioglossus and/or the respiratory rate of the patient may change in a characteristic manner relative to the amplitude and/or respiratory rate during normal breathing, while preserving the overall pattern of phasic and tonic activity that reflects the underlying respiratory drive. In some embodiments, the disordered breathing event detection algorithm is configured to identify a pattern in the EMG waveform indicative of the disordered breathing event. The disordered breathing event detection algorithm can implement any suitable technique, including, but not limited to rule-based systems, machine learning models (e.g., decision trees, support vector machines, naive Bayes classifiers, k-nearest neighbor algorithms, logistic regression, self-organized maps, fuzzy logic systems, data fusion processes, time-series forecasting, artificial neural networks such as deep learning networks, ensemble methods), or suitable combinations thereof. In embodiments in which the disordered breathing event detection algorithm comprises a machine learning model, the machine learning model can be trained on previous data of the patient and/or on data of one or more other patients. Optionally, the disordered breathing event detection algorithm can use other data types besides EMG data, such as motion data (described with reference to block 502b of FIG. 5) and/or other data (described with reference to block 502b of FIG. 5).

At block 1210, the method 1200 can include delivering stimulation energy, based on the detected disordered breathing event. In some embodiments, stimulation timing and/or stimulation parameters can be adjusted if a disordered breathing event is detected. For example, if the patient is continuing to have hypopneas, apneas, and/or other disordered breathing events while receiving stimulation energy, the parameters of the stimulation energy can be modified to increase an intensity of the stimulation energy to further protrude the tongue and dilate the airway during inspiration. Additionally or alternatively, a timing of the stimulation energy can be modified to ensure that the stimulation energy is delivered sooner before inspiration to prevent a disordered breathing event from occurring. In some embodiments, a timing of the stimulation energy can be modified based on the frequency of the detected disordered breathing events to deliver stimulation energy prior to the onset of the disordered breathing event. The detected disordered breathing events can provide a measure of efficacy of a neuromodulation therapy. In some embodiments, the detected disordered breathing events can be analyzed over time to identify trends in the disordered breathing events and relate the trends to the stimulation energy timing and/or parameters. This information can be used to control the stimulation energy. For example, if a patient continues to experience the same number or a greater number of disordered breathing events after receiving stimulation energy with certain parameters, the stimulation energy parameters can be further optimized until a desirable outcome, such as fewer disordered breathing events, is achieved. Still, in some embodiments, the method 1200 may omit block 1210 and does not modify the stimulation energy based on the detected disordered breathing events. In these embodiments, and others, the detected disordered breathing events can be provided to the patient and/or a clinician to provide insight into the state of the patient's sleep disordered breathing, an efficacy of the therapy, etc.

FIG. 13 is a flow diagram illustrating an example method 1300 for performing sleep state detection. The method 1300 can be performed as part of the sleep state detection 508 of FIG. 5. The method 1300 can be combined with any of the other methods described herein (e.g., the method 700 of FIG. 7 and/or the method 1200 of FIG. 12).

The method 1300 begins at block 1302 with obtaining EMG data including an EMG waveform. The EMG waveform can characterize activity of an anterior lingual muscle of the patient (e.g., the genioglossus). For example, the EMG data can be or include the ggEMG data 410 described above in connection with FIG. 4. The EMG data can be obtained using a sensor implanted in a sublingual region of a patient and/or a sensor positioned adjacent to and/or in contact with the anterior lingual muscle. According to some embodiments, the EMG data can be obtained from the conductive elements 114 of the neuromodulation device 100 described with reference to FIGS. 2A-3F.

At block 1304, the method 1300 can include determining an envelope of the EMG waveform. The envelope of the EMG waveform can be a smooth curve that outlines or generally follows the extremes of the waveform to provide useful information about the overall change in amplitude of the EMG data over time. For example, the envelope can be or include the ggEMG envelope 420 of FIG. 4. The envelope can be determined in accordance with the methods described above in connection with FIG. 5 (e.g., at block 504) and FIG. 6.

At block 1306, the method 1300 can optionally include extracting one or more features from the EMG waveform and/or the envelope of the EMG waveform. As noted with reference to block 504b of FIG. 5, a feature can comprise a measurable value, property, statistic, and/or transform EMG waveform and/or envelope. For example, the feature can comprise a measurable property of the EMG waveform and/or envelope representing a physiological event, such as onset of a certain portion of a respiratory cycle, a disordered breathing event, etc. In some embodiments, at least one feature is extracted from the EMG waveform. The EMG waveform can be the raw EMG waveform. Additionally or alternatively, the EMG waveform can be the cleaned EMG waveform, which can be obtained in accordance with the methods for generating cleaned EMG data described with reference to FIG. 6. The one or more features extracted at block 1306 can comprise a value of the EMG waveform, a magnitude of the EMG waveform, an amplitude of the EMG waveform, a frequency of the EMG waveform, a threshold crossing of the EMG waveform, a complexity of the EMG waveform, a range of the EMG waveform, a variance of the EMG waveform, a transform of the EMG waveform, a statistical parameter of the EMG waveform, and/or a temporal feature of the EMG waveform. In some embodiments, at least one feature is extracted from the envelope of the EMG waveform. For example, the at least one feature can comprise a value of the envelope, a magnitude of the envelope, an amplitude of the envelope, a frequency of the envelope, a threshold crossing of the envelope, a complexity of the envelope, a range of the envelope, a variance of the envelope, a transform of the envelope, a statistical parameter of the envelope, and/or a temporal feature of the envelope.

In some embodiments, the features of interest for the sleep state detection algorithm can differ from the features of interest for the breath detection algorithm, the breath prediction algorithm, and/or the disordered breathing event detection algorithm. In other embodiments, the features of interest for the sleep state detection algorithm can be the same as the features of interest for the breath detection algorithm, the breath prediction algorithm and/or the disordered breathing event detection algorithm. In still other embodiments, however, block 1306 is optional and may be omitted.

The features extracted at block 1306 for the sleep state detection can be computed over a time window. As previously noted with reference to block 508 of FIG. 5, the features for sleep state detection can be computed over a longer time window (e.g., a time window approximating the length of two, three, four, five, ten, or more respiratory cycles, such as a time window from about 30 seconds to about 60 seconds). The features can be computed over a time window of greater than or equal to about 30 seconds, about 40 seconds, about 50 seconds, about 60 seconds, about 70 seconds, about 80 seconds, about 90 seconds, about 100 seconds, about 110 seconds, or about 120 seconds.

At block 1308, the method 1300 can include identifying a sleep state of the patient, based on the envelope of the EMG waveform and/or the extracted features. The sleep state of the patient can be an indication of whether the patient is awake or asleep, whether the patient is in non-REM sleep or REM sleep, and/or whether the patient whether the patient is experiencing normal sleep or disordered sleep. The sleep state can be identified using a sleep state detection algorithm. The sleep state detection algorithm can be configured to use the EMG data to identify activity of the muscle indicative of the sleep state. For example, the amplitudes of tonic and phasic activity of the genioglossus can decrease during REM sleep, which may be reflected by changes in the characteristics of the EMG waveform. Differences in the amplitude and/or activation rate of genioglossus motor units can be reflected in spectral content and/or statistical distribution of the EMG waveform, which can be used to infer the patient's sleep state. Additionally or alternatively, changes in respiratory rate that are characteristic to the patient's sleep state can be reflected in a locally averaged respiratory rate derived from an envelope of the EMG waveform, and thus can be used to infer the patient's sleep state. The sleep state detection algorithm can implement any suitable technique, including, but not limited to rule-based systems, machine learning models (e.g., decision trees, support vector machines, naive Bayes classifiers, k-nearest neighbor algorithms, logistic regression, self-organized maps, fuzzy logic systems, data fusion processes, time-series forecasting, artificial neural networks such as deep learning networks, ensemble methods), or suitable combinations thereof. In embodiments in which the sleep state detection algorithm comprises a machine learning model, the machine learning model can be trained on previous data of the patient and/or on data of one or more other patients. Optionally, the sleep state detection algorithm can use other data types besides EMG data, such as motion data (described with reference to block 502b of FIG. 5) and/or other data (described with reference to block 502b of FIG. 5).

As previously noted, the features used to determine the sleep state can be computed over a larger time window, such as from about 30 seconds to about 60 seconds. However, sleep state can be detected for non-overlapping, discrete time windows each having a shorter length than the time window over which features are computed. For example, sleep state can be detected for a time window having a length of, for example, 5 seconds, 10 seconds, 15 seconds, 20 seconds, 25 seconds, or 30 seconds. Accordingly, the sleep state of a current time window can be determined based on features extracted from the current time window within which sleep state is to be detected, as well as features extracted from one or more previous time windows in which sleep state has already been detected. Additionally or alternatively, the detected sleep state of one or more previous time windows can be used to inform the sleep state detection of the present time window. Use of data and/or features collected over a longer time window allows for the determination of sleep state over a shorter time window. Because sleep states change more slowly than respiration, the data, features, and/or detected sleep state from previous time windows can help inform the detection of a present sleep state. For example, consider a scenario in which the data and/or features of a present time window indicate that the patient is in REM sleep. If the data and/or features from the previous three time windows indicate that the patient is in REM sleep, it is likely that the patient is still in REM sleep during the present time window and the detected sleep state is accurate. However, if the data and/or features from the previous three time windows indicate that the patient is in N1 sleep, it is unlikely that the patient is actually in REM sleep during the present time window, as sleep states normally progress from N1 sleep to N2 sleep to N3 sleep then to REM sleep. In such an example, the sleep state detection algorithm can discard the detected sleep state result for the present time window and/or can reanalyze the data and/or features of the present time window, using the information about the detected sleep state for the prior three time windows as feedback and/or in the new analysis. In some embodiments, sleep state can be detected continuously or near continuously.

At block 1310, the method 1300 can include delivering stimulation energy, based on the identified sleep state. For example, as described with reference to block 510a of FIG. 5, the sleep state detection can be used to determine whether or not to deliver stimulation energy. Delivering stimulation energy when the patient is awake can cause unnecessary discomfort in embodiments where the neuromodulation is intended to treat a sleep-related disorder. Accordingly, if the sleep state detection of block 1308 determines that the patient is awake, the stimulation energy can be prevented from being delivered. In contrast, if the sleep state detection of block 1308 determines that the patient is asleep, the stimulation energy can be delivered.

In some embodiments, for example, as described with reference to block 510b of FIG. 5, the identified sleep state can be used to control a timing of the stimulation energy (e.g., start time, end time, duration, duty cycle). As but one example, it may be advantageous to deliver stimulation energy to a hypoglossal nerve of a patient earlier before a predicted inspiration onset during deeper stages of sleep (e.g., N3 sleep, REM sleep), as the tone of the genioglossus is reduced during these stages of sleep.

According to various embodiments, for example as described with reference to block 510c of FIG. 5, the identified sleep state can be used to control parameters of the stimulation energy. Parameters of the stimulation energy can comprise, for example, amplitude, pulse width, duty cycle, polarity, frequency, ramp up time, ramp down time, ramp up rate, ramp down rate, and/or waveform. It may be beneficial to ramp up the intensity of the stimulation energy (e.g., increasing stimulation energy amplitude, increasing stimulation energy frequency, increasing stimulation energy pulse width) as the patient transitions from light sleep (e.g., N1 sleep, N2 sleep) into progressively deeper states of sleep (e.g., N3 sleep, REM sleep). Applying less intense stimulation energy during light sleep can prevent or limit the stimulation energy from arousing the patient and/or causing discomfort to the patient, while applying more intense stimulation energy during deep sleep can enhance an efficacy of the therapy. For example, during deeper sleep (e.g., N3 sleep, REM sleep) the tonic activity of the genioglossus can be lower than in light sleep (e.g., N1 sleep, N2 sleep) such that stimulation energy with higher intensity is required and/or desired during deep sleep to achieve a similar level of airway dilation as compared to when the patient is in light sleep.

FIG. 14 is a flow diagram illustrating an example method 1400 for detecting patient position during sleep. The method 1400 can be performed in connection with the workflow 500 of FIG. 5. The method 1400 can be combined with any of the other methods described herein (e.g., the method 700 of FIG. 7, the method 1200 of FIG. 12, and/or the method 1300 of FIG. 13).

The method 1400 begins at block 1402 with obtaining EMG data including an EMG waveform. The EMG waveform can characterize activity of an anterior lingual muscle of the patient (e.g., the genioglossus). For example, the EMG data can be or include the ggEMG data 410 described above in connection with FIG. 4. The EMG data can be obtained using a sensor implanted in a sublingual region of a patient and/or a sensor positioned adjacent to and/or in contact with the anterior lingual muscle. According to some embodiments, the EMG data can be obtained from the conductive elements 114 of the neuromodulation device 100 described with reference to FIGS. 2A-3F.

At block 1404, the method 1400 can include determining an envelope of the EMG waveform. The envelope of the EMG waveform can be a smooth curve that outlines or generally follows the extremes of the waveform to provide useful information about the overall change in amplitude of the EMG data over time. For example, the envelope can be or include the ggEMG envelope 420 of FIG. 4. The envelope can be determined in accordance with the methods described above in connection with FIG. 5 (e.g., at block 504) and FIG. 6.

At block 1406, the method 1400 can optionally include extracting one or more features from the EMG waveform and/or the envelope of the EMG waveform. As noted with reference to block 504b of FIG. 5, a feature can comprise a measurable value, property, statistic, and/or transform EMG waveform and/or envelope. For example, the feature can comprise a measurable property of the EMG waveform and/or envelope representing a physiological event, such as onset of a certain portion of a respiratory cycle, a disordered breathing event, etc. In some embodiments, at least one feature is extracted from the EMG waveform. The EMG waveform can be the raw EMG waveform. Additionally or alternatively, the EMG waveform can be the cleaned EMG waveform, which can be obtained in accordance with the methods for generating cleaned EMG data described with reference to FIG. 6. The one or more features extracted at block 1406 can comprise a value of the EMG waveform, a magnitude of the EMG waveform, an amplitude of the EMG waveform, a frequency of the EMG waveform, a threshold crossing of the EMG waveform, a complexity of the EMG waveform, a range of the EMG waveform, a variance of the EMG waveform, a transform of the EMG waveform, a statistical parameter of the EMG waveform, and/or a temporal feature of the EMG waveform. In some embodiments, at least one feature is extracted from the envelope of the EMG waveform. For example, the at least one feature can comprise a value of the envelope, a magnitude of the envelope, an amplitude of the envelope, a frequency of the envelope, a threshold crossing of the envelope, a complexity of the envelope, a range of the envelope, a variance of the envelope, a transform of the envelope, a statistical parameter of the envelope, and/or a temporal feature of the envelope.

In some embodiments, the features of interest for the patient position detection algorithm can differ from the features of interest for the breath prediction algorithm, the disordered breathing event detection algorithm, and/or the sleep state detection algorithm. In other embodiments, the features of interest for the patient position detection algorithm can be the same as the features of interest for the breath prediction algorithm, the disordered breathing event detection algorithm, and/or the sleep state detection algorithm. In still other embodiments, however, block 1406 is optional and may be omitted.

At block 1408, the method 1400 can include identifying a position of the patient during sleep (“sleep position”), based on the envelope of the EMG waveform and/or the extracted features. The sleep position can characterize the patient's entire body, e.g., a prone sleep position, a supine sleep position, and/or a lateral sleep position. In some embodiments, the sleep position characterizes a position and/or an orientation of the patient's head and/or shoulders. For example, the sleep position can characterize a nod angle of the patient's head, an axial angle of the patient's head, and/or a rotation of the patient's head. The sleep position can be detected using a sleep position detection algorithm. The sleep position detection algorithm can be configured to identify activity of the muscle indicative of the sleep position and/or a pattern in the EMG waveform indicative of the sleep position. For example, variations in the position of the patient's head in different sleep positions (e.g., supine, lateral) can distinctly influence tonic neck reflexes such that variations in head position are reflected as variations in the phasic and tonic activity of the genioglossus. The sleep position detection algorithm can implement any suitable technique, including, but not limited to rule-based systems, machine learning models (e.g., decision trees, support vector machines, naive Bayes classifiers, k-nearest neighbor algorithms, logistic regression, self-organized maps, fuzzy logic systems, data fusion processes, time-series forecasting, artificial neural networks such as deep learning networks, ensemble methods), or suitable combinations thereof. In embodiments in which the sleep position detection algorithm comprises a machine learning model, the machine learning model can be trained on previous data of the patient and/or on data of one or more other patients. Optionally, the sleep position detection algorithm can use other data types besides EMG data, such as motion data (described with reference to block 502b of FIG. 5) and/or other data (described with reference to block 502b of FIG. 5).

At block 1410, the method 1400 can include delivering stimulation energy, based on the detected sleep position. For example, the tendency for the upper airway to collapse is typically greater in the supine position than in the lateral position. Accordingly, if the method 1400 detects that the patient's sleep position is a lateral sleep position, the method 1400 can increase the stimulation energy delivered to the patient and/or increase the duration of the stimulation to counteract the tendency of the airway to collapse in the lateral sleeping position.

In some embodiments, the method 1400 can use the detected sleep position to control operation of an external system, such as external system 15. As previously described, the external system 15 can be configured to wirelessly transfer power to the implantable neuromodulation device 100. In some embodiments, the efficiency at which power is transferred from the external system 15 to the implantable neuromodulation device 100 can be based, at least in part, on the position and orientation of the patient (and thereby the implantable neuromodulation device 100) relative to the second antenna 12 of the external system 15. For example, if the patient is in a lateral sleep position, power may be transferred less efficiently to the implantable neuromodulation device 100. Accordingly, the external system 15 may increase the magnitude of the magnetic field emitted by the second antenna 12 to provide a consistent amount of power to the implantable neuromodulation device 100. Thus, the detected sleep position of the patient can be provided to the external system 15, the network 50, the patient device 70, the physician device 75, etc., to control operation of the external system 15.

Any of the algorithms described herein (e.g., the breath detection algorithm, the breath prediction algorithm, the sleep state detection algorithm, the disordered breathing event detection algorithm, the sleep position detection algorithm) can be updated. The algorithm can be updated based on any relevant data, such as data of the patient being monitored and/or treated, and/or data of other patients. The other patients can comprise a specific population of patients, such as patients with sleep disordered breathing, healthy patients, patients having similar characteristics as the patient being monitored (e.g., with respect to age, height, weight, body mass index, gender, diagnosis), etc.

Alternatively or in addition, an algorithm can be updated based on outputs from another algorithm. In one example, the respiration parameters of a breath prediction algorithm can be updated based on a detected sleep state of the patient. The phasic and tonic activity of the genioglossus muscle, for example, change between the various stages of sleep. Accordingly, updating the breath algorithms in accordance with the detected sleep state can improve an accuracy of the breath algorithms. Likewise, in some embodiments, the breath prediction algorithm can be updated based on the results of the disordered breathing event detection algorithm.

The updates can improve the accuracy of the algorithm. As but one example, if a patient's measured inter-breath interval is significantly different than the inter-breath interval respiration parameter of the breath prediction algorithm, the inter-breath interval respiration parameter of the breath prediction algorithm can be updated based on the patient's actual inter-breath interval, as detailed further above. In some examples, an average inter-breath interval can be determined from a population of patients, and the inter-breath interval respiration parameter of a breath prediction algorithm can be determined and/or updated to reflect the average inter-breath interval of the population. Updating the inter-breath interval respiration parameter of the breath prediction algorithm can allow the breath prediction algorithm to predict an upcoming inspiration onset more accurately, leading to a more effective therapy.

Algorithm updates can be determined using any suitable mechanism and/or any suitable source. In some embodiments, patient data can be communicated from an implantable device that collected the data to an external device or system (e.g., external device 11, external system 15, remote computing device 80, etc.) and/or to one or more servers (e.g., cloud servers), where the data can be processed to determine whether and/or how to update a relevant algorithm. For example, a processor of the external device can evaluate the patient data, determine whether and/or how to update the algorithm, and/or implement the algorithm updates. The updated algorithm can be communicated to the implantable device, which can execute the updated algorithm. Still, each of evaluation of the patient data, updating the algorithm, and executing the algorithm can be performed by any suitable device and may or may not be performed by the same device. Where and when these functions are performed can be based, at least in part, on a desired speed at which the functions are performed and/or the computational resources required to perform the functions as compared to the computational resources available on the device.

### CONCLUSION

Although many of the embodiments are described above with respect to systems, devices, and methods for modulation of a hypoglossal nerve of a patient for treating sleep disordered breathing, the technology is applicable to other applications and/or other approaches, such as modulation of other nerves of a patient and/or treatment of other diseases or conditions. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-14.

The various processes described herein can be partially or fully implemented using program code including instructions executable by one or more processors of a computing system for implementing specific logical functions or steps in the process. The program code can be stored on any type of computer-readable medium, such as a storage device including a disk or hard drive. Computer-readable media containing code, or portions of code, can include any appropriate media known in the art, such as non-transitory computer-readable storage media. Computer-readable media can include volatile and non-volatile, removable and non-removable media implemented in any method or technology for storage and/or transmission of information, including, but not limited to, random-access memory (RAM), read-only memory (ROM), electrically erasable programmable read-only memory (EEPROM), flash memory, or other memory technology; compact disc read-only memory (CD-ROM), digital video disc (DVD), or other optical storage; magnetic cassettes, magnetic tape, magnetic disk storage, or other magnetic storage devices; solid state drives (SSD) or other solid state storage devices; or any other medium which can be used to store the desired information and which can be accessed by a system device.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded.

To the extent any materials incorporated herein by reference conflict with the present disclosure, the present disclosure controls.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A method comprising:
obtaining EMG data using a sensor implanted in a sublingual region of a patient, wherein the EMG data comprises an EMG waveform indicative of activity of a muscle;
determining an envelope of the EMG waveform;
determining an inspiration onset of an upcoming respiratory cycle of the patient, based on the envelope of the EMG waveform; and
delivering stimulation energy to a hypoglossal nerve of the patient before the inspiration onset.

2-16. (canceled)

17. The method of claim 1, wherein determining the inspiration onset comprises predicting the inspiration onset; based on the envelope of the EMG waveform and using a breath prediction algorithm.

18. The method of claim 17, wherein the breath prediction algorithm is configured to predict the inspiration onset based on previous EMG data of at least one previous respiratory cycle of the patient.

19. The method of claim 18, wherein the breath prediction algorithm is configured to:
determine a time parameter for the at least one previous respiratory cycle, based on the previous EMG data, and
predict a time of the inspiration onset of the upcoming respiratory cycle based on the time parameter.

20. The method of claim 19, wherein the time parameter for the at least one previous respiratory cycle comprises one or more of the following: an inspiration onset time, an inspiration end time, an inspiration lag time, an expiration onset time, an expiration end time, an expiration lag time, or an inter-breath interval.

21. The method of claim 17, wherein the breath prediction algorithm is configured to:
detect a candidate breath of the at least one previous respiratory cycle,
determine whether the candidate breath was a valid breath, and
if the candidate breath was a valid breath, predict the inspiration onset based on a time parameter of the candidate breath.

22. The method of claim 17, wherein the breath prediction algorithm comprises a trained machine learning model.

23-24. (canceled)

25. The method of claim 22, wherein predicting the inspiration onset comprises inputting at least one feature extracted from the envelope of the EMG waveform into the trained machine learning model.

26. The method of claim 25, wherein the at least one feature extracted from the envelope of the EMG waveform comprises one or more of the following: a value of the envelope, a magnitude of the envelope, an amplitude of the envelope, a frequency of the envelope, a threshold crossing of the envelope, a complexity of the envelope, a range of the envelope, a variance of the envelope, a transform of the envelope, a statistical parameter of the envelope, or a temporal feature of the envelope.

27. The method of claim 17, further comprising:
assessing whether the predicted inspiration onset matched an actual inspiration onset of the upcoming respiratory cycle, and
adjusting the breath prediction algorithm based on the assessment.

28-29. (canceled)

30. The method of claim 1, wherein the stimulation energy is delivered at least 0.5 microseconds before the inspiration onset.

31-35. (canceled)

36. A system comprising:
a sensor configured to be implanted in a sublingual region of a patient;
an electrode configured to be implanted adjacent to a hypoglossal nerve of the patient and configured to deliver stimulation energy to the hypoglossal nerve;
one or more processors; and
a memory operably coupled to the one or more processors and storing instructions that, when executed by the processor, cause the system to perform operations comprising:
obtaining EMG data using the sensor, wherein the EMG data comprises an EMG waveform indicative of activity of a muscle,
determining an envelope of the EMG waveform,
determining an inspiration onset of an upcoming respiratory cycle of the patient, based on the envelope of the EMG waveform, and
delivering the stimulation energy via the electrode to the hypoglossal nerve before the inspiration onset.

37-49. (canceled)

50. The system of claim 36, wherein determining the inspiration onset comprises predicting the inspiration onset, based on the envelope of the EMG waveform and using a breath prediction algorithm.

51. The system of claim 50, wherein the breath prediction algorithm is configured to predict the inspiration onset based on previous EMG data of at least one previous respiratory cycle of the patient.

52. The system of claim 51, wherein the breath prediction algorithm is configured to:
determine a time parameter for the at least one previous respiratory cycle, based on the previous EMG data, and
calculate a time of the inspiration onset of the upcoming respiratory cycle based on the time parameter.

53. (canceled)

54. The system of claim 50, wherein the breath prediction algorithm is configured to:
detect a candidate breath of the at least one previous respiratory cycle,
determine whether the candidate breath was a valid breath, and
if the candidate breath was a valid breath, predict the inspiration onset based on a time parameter of the candidate breath.

55. The system of claim 50, wherein the breath prediction algorithm comprises a trained machine learning model.

56-57. (canceled)

58. The system of claim 55, wherein predicting the inspiration onset comprises inputting at least one feature extracted from the envelope of the EMG waveform into the trained machine learning model.

59. The system of claim 58, wherein the at least one feature extracted from the envelope of the EMG waveform comprises one or more of the following: a value of the envelope, a magnitude of the envelope, an amplitude of the envelope, a frequency of the envelope, a threshold crossing of the envelope, a complexity of the envelope, a range of the envelope, a variance of the envelope, a transform of the envelope, a statistical parameter of the envelope, or a temporal feature of the envelope.

60. The system of claim 50, wherein the operations further comprise:
assessing whether the predicted inspiration onset matched an actual inspiration onset of the upcoming respiratory cycle, and
adjusting the breath prediction algorithm based on the assessment.

61-71. (canceled)

72. A non-transitory computer-readable storage medium comprising instructions that, when executed by one or more processors of a computing system, cause the computing system to perform operations comprising:
obtaining EMG data using a sensor implanted in a sublingual region of a patient, wherein the EMG data comprises a EMG waveform indicative of activity of a muscle;
determining an envelope of the EMG waveform;
determining an inspiration onset of an upcoming respiratory cycle of the patient, based on the envelope of the EMG waveform; and
delivering stimulation energy to a hypoglossal nerve of the patient before the inspiration onset.

73-171. (canceled)
